(19) United States
(12) Patent Application Publication
(10) Pub. No.: US 2024/0132531 A1
(43) Pub. Date: Apr. 25, 2024

(54) NOVEL GALACTOSIDE INHIBITOR OF GALECTINS

(71) Applicant: GALECTO BIOTECH AB, Copenhagen N (DK)

(72) Inventors: Fredrik ZETTERBERG, Copenhagen N (DK); Kristoffer PETERSON, Copenhagen N (DK)

(73) Assignee: GALECTO BIOTECH AB, Copenhagen N (DK)

(21) Appl. No.: 18/257,382

(22) PCT Filed: Dec. 21, 2021
(86) PCT No.: PCT/EP2021/086975

§ 371 Date: Jun. 14, 2023

(30) Foreign Application Priority Data
Dec. 22, 2020 (EP) 20216480.2

(51) Int. Cl. C07H 19/056

(52) U.S. Cl. CPC C07H 19/056

(57) ABSTRACT

A D-galactopyranose compound of formula (1)

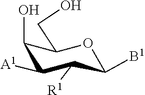

wherein
the pyranose ring is beta-D-galactopyranose, and these compounds are high affinity galectin-1 and/or 3 inhibitors for use in treatment of a disorder relating to the binding of a galectin-1 and/or 3 to a ligand in a mammal. The orders include: inflammation; fibrosis, endometriosis; scarring; keloid formation; aberrant scar formation; surgical adhesions; scleroderma; systemic sclerosis; septic shock; cancers; autoimmune diseases; metabolic disorders; coagulopathies; cardiovascular disorders; heart disease; heart failure; aortic stenosis; eye diseases; atherosclerosis; endocrine disorders; diabetes; insulin resistance; obesity; Diastolic HF; atrophic diseases; disorders related to transplantation in organs; acute burn; acute inflammatory reaction; chronic acute skin graft rejection; chronic scarring; asthma and other interstitial lung diseases; Otosclerosis, mesothelioma; post-surgery disorders; toxin exposure disorders; Tissue injury; Peripheral nerve repair; congenital hepatic fibrosis; hereditary fibrosing poikiloderma; liver disorders; neurodegenerative disorders.
metastasising cancers; autoimmune

### TECHNICAL FIELD

The present invention relates to novel compounds, the use of said compounds as medicament and for the manufacture of a medicament for the treatment of diseases or disorders such as but not limited to cancers; fibrosis; scarring; keloid formation; aberrant scar formation; surgical adhesions; pathological angiogenesis; eye diseases; HIV-1 diseases; inflammation or transplant rejection in mammals. The invention also relates to pharmaceutical compositions comprising said novel compounds.

### BACKGROUND ART

Galectins are proteins with a characteristic carbohydrate recognition domain (CRD). This is a tightly folded β-sandwich of about 130 amino acids (about 15 kDa) with the two defining features 1) a β-galactose binding site and 2) sufficient similarity in a sequence motif of about seven amino acids, most of which (about six residues) make up the β-galactose binding site. Galectins are synthesized as cytosolic proteins from where they can be targeted to the nucleus, specific cytososlic sites, or secreted to engage in mechanisms effecting physiological functions such as inflammation, immune responses, cell-migration and autophagy. (Johannes et. al 2018) There are now over 9319 publications on galectins in PubMed, with most, as mentioned above, about galectins-1 (>1989) and -3 (>4791). Evidence from literature suggests roles for galectins in e.g. fibrosis, inflammation and cancer (Dings et. al., Dubé-Delarosbil et. al 2017).

Galectin-1 is widely expressed in many cell types and tissues (www.proteinatlas.org) being involved in mechanisms such as apoptosis, adhesion and migration, cell transformation, invasion and metastasis immune escape and angiogenesis. Upregulation of galectin 1 has also been associated with cancer (Dings et. al. 2018), inflammation (Sundblad et. al., 2017) fibrotic disease (Kathiriya et. al 2017, Wu et. al. 2019 and Bennet et. al 2019) and diabetes (Drake et. al. 2022). Example of small molecule ligands including β-D-galactopyranoside were recently reviewed and exemplified in Blanchard et. al 2016 and Sethi et. al 2021).

Galectin-3 is widely expressed in many cell types and tissues (www.proteinatlas.org) being involved in mechanisms such as apoptosis, adhesion and migration, cell transformation, invasion and metastasis immune escape and angiogenesis. Upregulation of galectin 3 has also been associated with cancer, inflammation, neurodegenerative disease, fibrotic disease and diabetes (Dings et. al. 2018, Slack et. al. 2020, Li et. al. 2016) Example of small molecule ligands including β-D-galactopyranoside were recently reviewed and exemplified in Blanchard et. al 2014 and Sethi et. al 2021.

### SUMMARY OF THE INVENTION

The compounds of the present invention are novel β-D-galactopyranose compounds that unexpectedly have shown high affinity for galectin-1 and/or -3 and are considered novel potent drug candidates.

In a first aspect the present invention relates to a D-galactopyranose compound of formula (1)

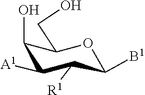

wherein
the pyranose ring is β-D-galactopyranose,
A¹ is

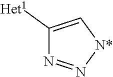

wherein the asterix * indicates the nitrogen atom of the triazole ring that is covalently attached to the galactopyranose;
wherein Het¹ is a five or six membered heteroaromatic ring selected from the group consisting of formulas 2 to 11, wherein the asterix * indicates the carbon or nitrogen atom of the heteroaromatic ring that is covalently attached to the triazole group in formula A¹:

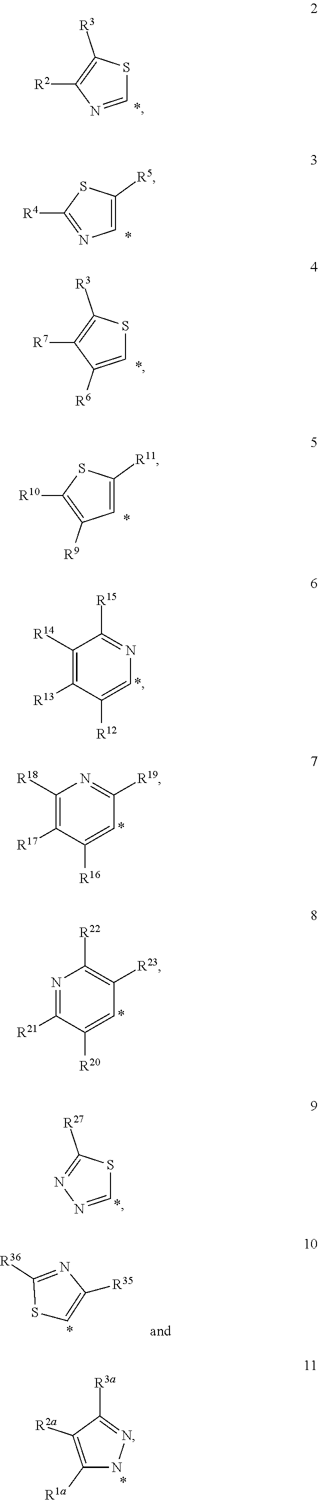

wherein R^{1a}, R^{2a}, R^{3a}, R², R³, R⁴, R⁵ R⁶, R⁷, R⁸, R⁹ R¹⁰, R¹¹, R¹², R¹³, R¹⁴, R¹⁵, R¹⁶, R¹⁷, R¹⁸, R¹⁹, R²⁰, R²¹, R²² and R²³, R²⁷, R³⁵ and R³⁶ are independently selected from H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
wherein B¹ is a pyrazol substituted with one or more groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R²⁸—C₁₋₆ alkyl, c) C₃₋₆ cycloalkyl optionally substituted with one or more of C₁₋₆ alkyl, amino, CN, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, i) C₂-alkynyl, j) R²⁸,
wherein R²⁸ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C2-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R³³)(R³⁴)N; or b) (C₁₋₆ alkyl-SO₂)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R²⁹ is hydrogen or C₁₋₆ alkyl;
R₃₀ is hydrogen or C₁₋₆ alkyl; or
(R²⁹)(R³⁰)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R³¹ is hydrogen, C₁₋₆ alkyl, C₁₋₆ alkylcarbonyl, or C₁₋₆ alkylsulfonyl;
R³² is hydrogen or C₁₋₆ alkyl; or
(R³¹)(R³²)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R³³ is hydrogen, C₁₋₆ alkyl, C₁₋₆ alkylcarbonyl, or C₁₋₆ alkylsulfonyl;
R³⁴ is hydrogen or C₁₋₆ alkyl; or
(R³³)(R³⁴)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
k) halogen, and 1) cyano;
R¹ is selected from the group consisting of a) OC₁₋₆ alkyl optionally substituted with one or more halogen, phenyl, phenyl substituted with one or more groups selected form OH and halogen, CN, OR³⁷, NR³⁸R³⁹, and CONH₂, wherein R³⁷ is selected from the group consisting of H, CN, a halogen, methyl optionally substituted with a F, OCH₃ optionally substituted with a F, OCH₂CH₃ optionally substituted with a F, OH, and R⁴⁰—CONH— wherein R⁴⁰ is selected from C₁₋₃ alkyl and cyclopropyl, R³⁸ is selected from the group consisting of H, CN, a halogen, methyl optionally substituted with a F, OCH₃ optionally substituted with a F, OCH₂CH₃ optionally substituted with a F, OH, and R⁴¹—CONH— wherein R⁴¹ is selected from C₁₋₃ alkyl and cyclopropyl, and R³⁹ is selected from the group consisting of H, CN, a halogen, methyl optionally substituted with a F, OCH₃ optionally substituted with a F, OCH₂CH₃ optionally substituted with a F, OH, and R⁴²—CONH— wherein R⁴² is selected from C₁₋₃ alkyl and cyclopropyl, b) branched OC₃₋₆ alkyl optionally substituted with one or more halogen, CN, OR⁴³, NR⁴⁴R⁴⁵, and CONH₂, wherein R⁴³ is selected from the group consisting of H, CN, a halogen, methyl optionally substituted with a F, OCH₃ optionally substituted with a F, OCH₂CH₃ optionally substituted with a F, OH, and R⁴⁶—CONH— wherein R⁴⁶ is selected from C₁₋₃ alkyl and cyclopropyl, R⁴⁴ is selected from the group consisting of H, CN, a halogen, methyl optionally substituted with a F, OCH₃ optionally substituted with a F, OCH₂CH₃ optionally substituted with a F, OH, and R⁴⁷—CONH— wherein R⁴⁷ is selected from C₁₋₃ alkyl and cyclopropyl, and R⁴⁵ is selected from the group consisting of H, CN, a halogen, methyl optionally substituted with a F, OCH₃ optionally substituted with a F, OCH₂CH₃ optionally substituted with a F, OH, and R⁴⁸—CONH— wherein R⁴⁸ is selected from C₁₋₃ alkyl and cyclopropyl, c) cyclic OC₃₋₆ alkyl optionally substituted with one or more halogen, CN, OR⁴⁹, NR⁵⁰R⁵¹, and CONH₂, wherein R⁴⁹ is selected from the group consisting of H, CN, a halogen, methyl optionally substituted with a F, OCH₃ optionally substituted with a F, OCH₂CH₃ optionally substituted with a F, OH, and R⁵²—CONH— wherein R⁵² is selected from C₁₋₃ alkyl and cyclopropyl, R⁵⁰ is selected from the group consisting of H, CN, a halogen, methyl optionally substituted with a F, OCH₃ optionally substituted with a F, OCH₂CH₃ optionally substituted with a F, OH, and R⁵³—CONH— wherein R⁵³ is selected from C₁₋₃ alkyl and cyclopropyl, and R⁵¹ is selected from the group consisting of H, CN, a halogen, methyl optionally substituted with a F, OCH₃ optionally substituted with a F, OCH₂CH₃ optionally substituted with a F, OH, and R⁵⁴—CONH— wherein R⁵⁴ is selected from C₁₋₃ alkyl and cyclopropyl, d) OH; or
a pharmaceutically acceptable salt or solvate thereof.

In second aspect the present invention concerns a β-D-galactopyranose compound of formula (1)

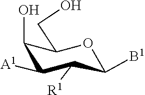

wherein
the pyranose ring is β-D-galactopyranose,
A1 is

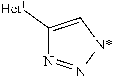

wherein the asterix * indicates the nitrogen atom of the triazole ring that is covalently attached to the galactopyranose;
wherein Het¹ is a five or six membered heteroaromatic ring selected from the group consisting of formulas 2 to 11, wherein the asterix * indicates the carbon or nitrogen atom of the heteroaromatic ring that is covalently attached to the triazole group in formula A¹:

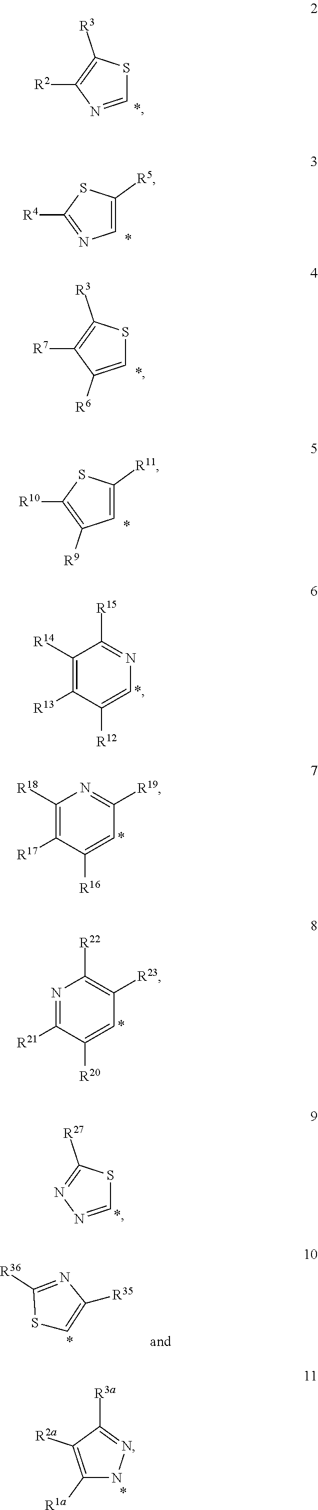

wherein R^{1a}, R^{2a}, R^{3a}, R², R³, R⁴, R⁵ R⁶, R⁷, R⁸, R⁹ R¹⁰, R¹¹, R¹², R¹³, R¹⁴, R¹⁵, R¹⁶, R¹⁷, R¹⁸, R¹⁹, R²⁰, R²¹, R²² and R²³, R²⁷, R³⁵ and R³⁶ are independently selected from H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁_alkyl;
wherein B¹ is a pyrazol substituted with one or more groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R²⁸—C₁₋₆ alkyl, c) C₃₋₆ cycloalkyl optionally substituted with one or more of C₁₋₆ alkyl, amino, CN, halogen, or hydroxy, c) C₁₋₆ alkenyl, d) C₁ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, i) C₂-alkynyl, and j) R²⁸;
wherein R²⁸ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C2-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R³³)(R³⁴)N; or b) (C₁₋₆ alkyl-SO₂)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R²⁹ is hydrogen or C₁₋₆ alkyl;
R₃₀ is hydrogen or C₁₋₆ alkyl; or
(R²⁹)(R³⁰)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R³¹ is hydrogen, C₁₋₆ alkyl, C₁₋₆ alkylcarbonyl, or C₁₋₆ alkylsulfonyl;
R³² is hydrogen or C₁₋₆ alkyl; or
(R³¹)(R³²)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R³³ is hydrogen, C₁₋₆ alkyl, C₁₋₆ alkylcarbonyl, or C₁₋₆ alkylsulfonyl;
R³⁴ is hydrogen or C₁₋₆ alkyl; or
(R³³)(R³⁴)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹ is selected from the group consisting of a) OC₁₋₆ alkyl optionally substituted with one or more halogen, phenyl, phenyl substituted with one or more groups selected form OH and halogen, CN, OR³⁷, NR³⁸R³⁹, and CONH₂, wherein R³⁷ is selected from the group consisting of H, CN, a halogen, methyl optionally substituted with a F, OCH₃ optionally substituted with a F, OCH₂CH₃ optionally substituted with a F, OH, and R⁴⁰—CONH— wherein R⁴⁰ is selected from C₁₋₃ alkyl and cyclopropyl, R³⁸ is selected from the group consisting of H, CN, a halogen, methyl optionally substituted with a F, OCH₃ optionally substituted with a F, OCH₂CH₃ optionally substituted with a F, OH, and R⁴¹—CONH— wherein R⁴¹ is selected from C₁₋₃ alkyl and cyclopropyl, and R³⁹ is selected from the group consisting of H, CN, a halogen, methyl optionally substituted with a F, OCH₃ optionally substituted with a F, OCH₂CH₃ optionally substituted with a F, OH, and R⁴²—CONH— wherein R⁴² is selected from C₁₋₃ alkyl and cyclopropyl, b) branched OC₃₋₆ alkyl optionally substituted with one or more halogen, CN, OR⁴³, NR⁴⁴R⁴⁵, and CONH₂, wherein R⁴³ is selected from the group consisting of H, CN, a halogen, methyl optionally substituted with a F, OCH₃ optionally substituted with a F, OCH₂CH₃ optionally substituted with a F, OH, and R⁴⁶—CONH— wherein R⁴⁶ is selected from C₁₋₃ alkyl and cyclopropyl, R⁴⁴ is selected from the group consisting of H, CN, a halogen, methyl optionally substituted with a F, OCH₃ optionally substituted with a F, OCH₂CH₃ optionally substituted with a F, OH, and R⁴⁷—CONH— wherein R⁴⁷ is selected from C₁₋₃ alkyl and cyclopropyl, and R⁴⁵ is selected from the group consisting of H, CN, a halogen, methyl optionally substituted with a F, OCH₃ optionally substituted with a F, OCH₂CH₃ optionally substituted with a F, OH, and R⁴⁸—CONH— wherein R⁴⁸ is selected from C₁₋₃ alkyl and cyclopropyl, c) cyclic OC₃₋₆ alkyl optionally substituted with one or more halogen, CN, OR⁴⁹, NR⁵⁰R⁵¹, and CONH₂, wherein R⁴⁹ is selected from the group consisting of H, CN, a halogen, methyl optionally substituted with a F, OCH₃ optionally substituted with a F, OCH₂CH₃ optionally substituted with a F, OH, and R⁵²—CONH— wherein R⁵² is selected from C₁₋₃ alkyl and cyclopropyl, R⁵⁰ is selected from the group consisting of H, CN, a halogen, methyl optionally substituted with a F, OCH₃ optionally substituted with a F, OCH₂CH₃ optionally substituted with a F, OH, and R⁵³—CONH— wherein R⁵³ is selected from C₁₋₃ alkyl and cyclopropyl, and R⁵¹ is selected from the group consisting of H, CN, a halogen, methyl optionally substituted with a F, OCH₃ optionally substituted with a F, OCH₂CH₃ optionally substituted with a F, OH, and R⁵⁴—CONH— wherein R⁵⁴ is selected from C₁₋₃ alkyl and cyclopropyl; or
a pharmaceutically acceptable salt or solvate thereof.

In an embodiment Het1 is selected from the group consisting of

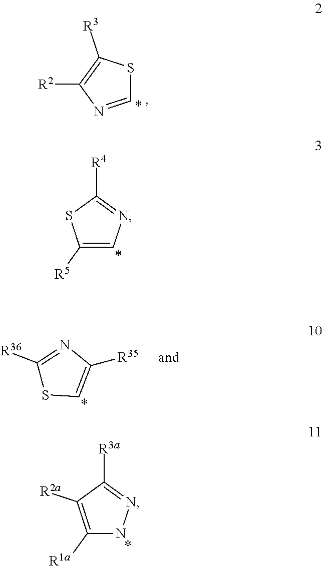

wherein R² is selected from the group consisting of hydrogen, methyl, OH and halogen;
R³ is selected from the group consisting of hydrogen, C₁₋₆ alkyl and halogen;
R⁴ is selected from the group consisting of OH, C₁₋₆ alkyl, halogen and amino;
R⁵ is selected from the group consisting of hydrogen, C₁₋₆ alkyl and halogen;
R³⁵ and R³⁶ are independently selected from hydrogen, C₁₋₆ alkyl, amino and halogen.

In a further embodiment Het1 is formula 2
wherein R² is selected from the group consisting of hydrogen, methyl, OH and halogen; and R³ is selected from the group consisting of hydrogen, methyl and halogen.

In a still further embodiment B¹ is a pyrazolyl substituted with one or more groups selected from the group consisting of a) C₁₋₄ alkyl optionally substituted with one or more of C₁₋₆ alkyl, amino, CN, halogen, hydroxy, C₁₋₆ alkoxy, carboxy, alkoxycarbonyl, H₂NCO, b) R²⁸—C₁₋₆ alkyl, c) C₃₋₆ cycloalkyl optionally substituted with one or more of C₁₋₆ alkyl, amino, CN, halogen, or hydroxy, c) C₂₋₄ alkenyl, d) C₁₋₆ alkoxy, e) C₁₋₆ alkylthio, f) C₁₋₆ alkylsulfonyl, g) COOH or COOC₁₋₄ alkyl h) (R³¹)(R³²)N, i) C₂-alkynyl, j) R²⁸, k) halogen, l) cyano; wherein R²⁸, R³¹ and R³² are as defined in the first aspect.

In a still further embodiment B¹ is a pyrazolyl substituted with one or more groups selected from the group consisting of pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R²⁸-C₁₋₆ alkyl, c) C₃₋₆ cycloalkyl optionally substituted with one or more of C₁₋₆ alkyl, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, and j) R²⁸;
wherein R²⁸ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R³³)(R³⁴)N; or b) (C₁₋₆ alkyl-SO₂)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R²⁹ is hydrogen or C₁₋₆ alkyl;
R₃₀ is hydrogen or C₁₋₆ alkyl; or
(R²⁹)(R³⁰)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R³¹ is hydrogen, C₁₋₆ alkyl, C₁₋₆ alkylcarbonyl, or C₁₋₆ alkylsulfonyl;
R³² is hydrogen or C₁₋₆ alkyl; or
(R³¹)(R³²)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R³³ is hydrogen, C₁₋₆ alkyl, C₁₋₆ alkylcarbonyl, or C₁₋₆ alkylsulfonyl;
R³⁴ is hydrogen or C₁₋₆ alkyl; or
(R³³)(R³⁴)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl.

In a further embodiment B¹ is a pyrazolyl substituted with a phenyl optionally substituted with a group selected from methyl, CF₃, and halogen.

In a still further embodiment B¹ is

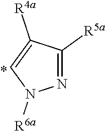

wherein the asterix * indicates the carbon atom of the pyrazol ring that is covalently attached to the galactopyranose,
wherein R^{4a}, R^{5a}, and R^{6a} are independently selected from the group consisting of hydrogen, C₁₋₄ alkyl, C₂₋₄ alkenyl, halogen, cyano, COOH, COOC₁₋₄ alkyl, pyrrolyl, furanyl, thienyl, pyrazolyl, isoxazolyl, isothiazolyl, imidazolyl, oxazolyl, thiazolyl, triazolyl, oxadiazolyl, thiadiazolyl, pyridinyl, benzothiazolyl, phenyl, or indolyl, provided that not all three of R^{4a}, R^{5a}, and R^{6a} are hydrogen at the same time; wherein pyrrolyl, furanyl, thienyl, pyrazolyl, isoxazolyl, isothiazolyl, imidazolyl, oxazolyl, thiazolyl, triazolyl, oxadiazolyl, thiadiazolyl, pyridinyl, benzothiazolyl, phenyl, or indolyl are optionally substituted with a group selected from cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂.

In a still further embodiment B¹ is

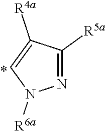

wherein the asterix * indicates the carbon atom of the pyrazol ring that is covalently attached to the galactopyranose,
wherein R^{4a}, R^{5a}, and R^{6a} are independently selected from the group consisting of hydrogen, pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl, provided that not all three of R^{4a}, R^{5a}, and R^{6a} are hydrogen at the same time; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R²⁸-C₁₋₆ alkyl, c) C₃₋₆ cycloalkyl optionally substituted with one or more of C₁₋₆ alkyl, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, and j) R²⁸; wherein R²⁸, R²⁹, R³⁰, R³¹ and R³² are as defined above. Typically, R^{4a} is hydrogen, R^{5a} is selected from hydrogen, halogen, C₁₋₃ alkyl, COOH, COOC₁₋₃ alkyl, C₂₋₃ alkenyl, cyano and R^{6a} is selected from a) a phenyl substituted with a group selected from methyl, CF₃, and halogen; b) a pyridinyl substituted with a group selected from methyl, CF₃, and halogen; or c) a benzothiazolyl substituted with a group selected from methyl, CF₃, and halogen. In an alternative, R^{4a} is hydrogen, R^{5a} is hydrogen and R^{6a} is a phenyl substituted with a group selected from methyl, CF₃, and halogen.

In a still further embodiment R¹ is selected from OC₁₋₆ alkyl optionally substituted with one or more halogen, phenyl, phenyl substituted with one or more groups selected form OH and halogen, CN, OR³⁷, NR³⁸R³⁹, and CONH₂, wherein R³⁷ is selected from the group consisting of H, CN, a halogen, methyl optionally substituted with a F, OCH₃ optionally substituted with a F, OCH₂CH₃ optionally substituted with a F, OH, and R⁴⁰—CONH— wherein R⁴⁰ is selected from C₁₋₃ alkyl and cyclopropyl, R³⁸ is selected from the group consisting of H, CN, a halogen, methyl optionally substituted with a F, OCH₃ optionally substituted with a F, OCH₂CH₃ optionally substituted with a F, OH, and R⁴¹—CONH— wherein R⁴¹ is selected from C₁₋₃ alkyl and cyclopropyl, and R³⁹ is selected from the group consisting of H, CN, a halogen, methyl optionally substituted with a F, OCH₃ optionally substituted with a F, OCH₂CH₃ optionally substituted with a F, OH, and R⁴²—CONH— wherein R⁴² is selected from C₁₋₃ alkyl and cyclopropyl.

In a further embodiment R¹ is selected from OC₁₋₆ alkyl optionally substituted with one or more halogen.

In a still further embodiment R¹ is selected from OC₁₋₃ alkyl.

In a further embodiment R¹ is OH.

Preferably, the compound of formula (1) selected from any one of the group consisting of:
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
5-Chloro-1-{5-{3-deoxy-2-O-methyl-3-[4-(2-thiazolyl)-1H-1,2,3-triazol-1-yl]-β-D-galactopyranosyl}-1H-1,2-pyrazol-1-yl}-2-(trifluoromethyl)benzene,
5-Chloro-1-{5-{3-deoxy-3-[4-(5-methylthiazol-2-yl)-1H-1,2,3-triazol-1-yl]-2-O-methyl-β-D-galactopyranosyl}-1H-1,2-pyrazol-1-yl}-2-(trifluoromethyl)benzene, and
5-Chloro-1-{5-{3-deoxy-3-[4-(4-methylthiazol-2-yl)-1H-1,2,3-triazol-1-yl]-2-O-methyl-β-D-galactopyranosyl}-1H-1,2-pyrazol-1-yl}-2-(trifluoromethyl)benzene; or a pharmaceutically acceptable salt or solvate thereof.

In a further embodiment, the compound of formula (1) selected from any one of the group consisting of:
5-Chloro-1-{5-{3-deoxy-3-[4-(2-thiazolyl)-1H-1,2,3-triazol-1-yl]-β-D-galactopyranosyl}-1H-1,2-pyrazol-1-yl}-2-(trifluoromethyl)benzene,
1-{5-{3-[4-(2-Aminothiazol-4-yl)-1H-1,2,3-triazol-1-yl]-3-deoxy-β-D-galactopyranosyl}-1H-1,2-pyrazol-1-yl}-5-chloro-2-(trifluoromethyl)benzene,
5-Chloro-1-{5-{3-deoxy-3-[4-(2-hydroxythiazol-4-yl)-1H-1,2,3-triazol-1-yl]-β-D-galactopyranosyl}-1H-1,2-pyrazol-1-yl}-2-(trifluoromethyl)benzene,
1-{5-{3-[4-(3-Chloro-1H-1,2-pyrazol-1-yl)-1H-1,2,3-triazol-1-yl]-3-deoxy-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-β-D-galactopyranosyl}-3-methyl-1H-1,2-pyrazol-1-yl}-5-chloro-2-(trifluoromethyl)benzene,
1-{5-{3-[4-(5-Chlorothiazol-2-yl)-1H-1,2,3-triazol-1-yl]-3-deoxy-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-β-D-galactopyranosyl}-1H-1,2-pyrazol-1-yl}-5-chloro-2-(trifluoromethyl)benzene,
1-{5-{3-[4-(5-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-methyl-1H-1,2-pyrazol-1-yl}-5-chloro-2-(trifluoromethyl)benzene,
5-Chloro-1-{5-{3-deoxy-2-O-methyl-3-[4-(2-thiazolyl)-1H-1,2,3-triazol-1-yl]-β-D-galactopyranosyl}-3-methyl-1H-1,2-pyrazol-1-yl}-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-methyl-1H-1,2-pyrazol-1-yl}-2-methylbenzothiazole,
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-2-methylbenzothiazole,
1-{5-{3-[4-(4-Chlorothiazol-2-yl)-1H-1,2,3-triazol-1-yl]-3-deoxy-2-O-methyl-β-D-galactopyranosyl}-3-ethoxycarbonyl-1H-1,2-pyrazol-1-yl}-5-chloro-2-(trifluoromethyl)benzene,
1-{3-Carboxy-5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
5-Bromo-1-{5-{3-[4-(4-chlorothiazol-2-yl)-1H-1,2,3-triazol-1-yl]-3-deoxy-β-D-galactopyranosyl}-1H-1,2-pyrazol-1-yl}-2-(trifluoromethyl)benzene,
5-Bromo-1-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-4,5-dichloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-4-fluoro-2-(trifluoromethyl)benzene,
5-Bromo-1-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-4-fluoro-2-(trifluoromethyl)benzene,
5-Bromo-1-{5-{3-[4-(4-chlorothiazol-2-yl)-1H-1,2,3-triazol-1-yl]-3-deoxy-β-D-galactopyranosyl}-1H-1,2-pyrazol-1-yl}-4-fluoro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-chloro-1H-1,2-pyrazol-1-yl}-5-chloro-2-(trifluoromethyl)benzene,
1-{3-Bromo-5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-(1-methylethenyl)-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-isopropyl-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-ethenyl-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-ethyl-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-cyano-1H-1,2-pyrazol-1-yl}-5-chloro-2-(trifluoromethyl)benzene,
3-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)pyridine,
5-Bromo-3-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-2-(trifluoromethyl)pyridine,
1-{5-{3-[4-(2-Aminothiazol-4-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
5-Chloro-1-{5-{3-deoxy-3-[4-(2-hydroxythiazol-4-yl)-1H-1,2,3-triazol-1-yl]-2-O-methyl-β-D-galactopyranosyl}-1H-1,2-pyrazol-1-yl}-2-(trifluoromethyl)benzene,
4,5-Dichloro-1-{5-{3-deoxy-3-[4-(2-hydroxythiazol-4-yl)-1H-1,2,3-triazol-1-yl]-2-O-methyl-β-D-galactopyranosyl}-1H-1,2-pyrazol-1-yl}-2-(trifluoromethyl)benzene,
5-Bromo-1-{5-{3-deoxy-3-[4-(2-hydroxythiazol-4-yl)-1H-1,2,3-triazol-1-yl]-2-O-methyl-β-D-galactopyranosyl}-1H-1,2-pyrazol-1-yl}-4-fluoro-2-(trifluoromethyl)benzene,
5-Chloro-1-{5-{3-deoxy-2-O-methyl-3-[4-(4-thiazolyl)-1H-1,2,3-triazol-1-yl]-β-D-galactopyranosyl}-1H-1,2-pyrazol-1-yl}-2-(trifluoromethyl)benzene, and
1-{3-Aminocarbonyl-5-{3-[4-(4-chlorothiazol-2-yl)-1H-1,2,3-triazol-1-yl]-3-deoxy-2-O-methyl-3-D-galactopyranosyl}-1H-1,2-pyrazol-1-yl}-5-chloro-2-(trifluoromethyl)benzene; or a pharmaceutically acceptable salt or solvate thereof.

In a further aspect the present invention relates to a compound of formula (1) for use as a medicine.

In a still further aspect, the present invention relates to a pharmaceutical composition comprising the compound of any one of the previous claims and optionally a pharmaceutically acceptable additive, such as a carrier and/or excipient.

In a further aspect the present invention relates to a compound of formula (1) of the present invention for use in a method for treating a disorder relating to the binding of a galectin-1 and/or -3 to a ligand in a mammal, such as a human.

In a further embodiment the disease or disorder is selected from the group consisting of inflammation, such as acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, (e.g. lung, liver, kidney, heart, skin, muscle, gut), chronic bacterial infections, chronic viral related inflammation; fibrosis, such as pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust (such as dust from terror attack known as 9/11), acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; coagulopathies, such as thrombosis proneness idiopathic (thrombophilia), autoimmune based thrombophilia, microthrombosis at multiorgan failure, COVID-19 related coagulopathy, thrombophilia in cancer disease; cardiovascular disorders, such as cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; endocrine disorders, such as Addison, autoimmune hypophysitis; metabolic diseases such as diabetes; type 2 diabetes; insulin resistance; obesity; Diastolic HF; atrophic diseases in the brain, such as Alzheimer's and Parkinson's, atrophic diseases in the cerebellum, such as cerebellar atrophy, atrophic spinal diseases such as ALS; disorders related to transplantation in organs, such as anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis such as COPD (Chronic Obstructive Pulmonary Disease) and asthma; Otosclerosis, mesothelioma; post-surgery disorders, such as anti-keloid, anti-stricture, anti-adhesion, anti-thrombosis, fibrosis/scar reduction following cosmetic procedures; toxin exposure disorders, such as toxic hepatitis, cholera toxin related, mushroom toxin based acute renal failure, pertussis toxin, Aeromonas hydrophila enterotoxin, cadmium induced cardiac toxicity, Helicobacter O-antigen related toxicity, LPS based toxicity, Streptozotocin toxicity, asbestos exposure, Nephrogenic Systemic Fibrosis (Post Contrast Agents); Tissue injury, such as Spinal cord injury, Peripheral nerve repair; congenital hepatic fibrosis; hereditary fibrosing poikiloderma with tendon contractures, myopathy, and pulmonary fibrosis; liver disorders, such as non-alcoholic steatohepatitis (NASH) or non-alcoholic fatty liver disease, liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease; Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis (ALS), multiple sclerosis (MS), peripheral nephropathy.

In a still further aspect the present invention relates to a method for treatment of a disease or disorder relating to the binding of a galectin-1 and/or -3 to a ligand in a mammal, such as a human, wherein a therapeutically effective amount of at least one compound of formula (1) of the present invention is administered to a mammal in need of said treatment.

In a further embodiment the disease or disorder is selected from the group consisting of inflammation, such as acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, (e.g. lung, liver, kidney, heart, skin, muscle, gut), chronic bacterial infections, chronic viral related inflammation; fibrosis, such as pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust (such as dust from terror attack known as 9/11), acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; coagulopathies, such as thrombosis proneness idiopathic (thrombophilia), autoimmune based thrombophilia, microthrombosis at multiorgan failure, COVID-19 related coagulopathy, thrombophilia in cancer disease; cardiovascular disorders, such as cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; endocrine disorders, such as Addison, autoimmune hypophysitis; metabolic diseases such as diabetes; type 2 diabetes; insulin resistance; obesity; Diastolic HF; atrophic diseases in the brain, such as Alzheimer's and Parkinson's, atrophic diseases in the cerebellum, such as cerebellar atrophy, atrophic spinal diseases such as ALS; disorders related to transplantation in organs, such as anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis such as COPD (Chronic Obstructive Pulmonary Disease) and asthma; Otosclerosis, mesothelioma; post-surgery disorders, such as anti-keloid, anti-stricture, anti-adhesion, anti-thrombosis, fibrosis/scar reduction following cosmetic procedures; toxin exposure disorders, such as toxic hepatitis, cholera toxin related, mushroom toxin based acute renal failure, pertussis toxin, Aeromonas hydrophila enterotoxin, cadmium induced cardiac toxicity, Helicobacter O-antigen related toxicity, LPS based toxicity, Streptozotocin toxicity, asbestos exposure, Nephrogenic Systemic Fibrosis (Post Contrast Agents); Tissue injury, such as Spinal cord injury, Peripheral nerve repair; congenital hepatic fibrosis; hereditary fibrosing poikiloderma with tendon contractures, myopathy, and pulmonary fibrosis; liver disorders, such as non-alcoholic steatohepatitis (NASH) or non-alcoholic fatty liver disease, liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease; Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis (ALS), multiple sclerosis (MS), peripheral nephropathy.

Another aspect of the present invention concerns combination therapy involving administering a compound of formula (1) of the present invention together with a therapeutically active compound different from the compound of formula (1) (interchangeable with “a different therapeutically active compound”). In one embodiment the present invention relates to a combination of a compound of formula (I) and a different therapeutically active compound for use in treatment of a disorder relating to the binding of a galectin-1/3 to a ligand in a mammal. Such disorders are disclosed below.

In an embodiment of the present invention, a therapeutically effective amount of at least one compound of formula (I) of the present invention is administered to a mammal in need thereof in combination with a different therapeutically active compound. In a further embodiment, said combination of a compound of formula (I) together with a different therapeutically active compound is administered to a mammal suffering from a disorder selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis or non-alcoholic fatty liver disease.

A non-limiting group of cancers given as examples of cancers, including both solid and liquid cancers, that may be treated, managed and/or prevented by administration of a compound of formula (1) in combination with a different therapeutically active compound is selected from: colon carcinoma, breast cancer, head and neck cancer, testis cancer, urothelial cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

In some aspects of the present invention, the administration of at least one compound of formula (I) of the present invention and at least one additional therapeutic agent demonstrates therapeutic synergy. In some aspects of the methods of the present invention, a measurement of response to treatment observed after administering both at least one compound of formula (I) of the present invention and the additional therapeutic agent is improved over the same measurement of response to treatment observed after administering either the at least one compound of formula (I) of the present invention or the additional therapeutic agent alone.

A further aspect of the present invention concerns combination therapy involving administering a compound of formula (I) of the present invention together with an anti-fibrotic compound different from the compound of formula (I) to a mammal in need thereof. In a further embodiment, such anti-fibrotic compound may be selected from the following non-limiting group of anti-fibrotic compounds: pirfenidone, nintedanib, simtuzumab (GS-6624, AB0024), BG00011 (STX100), PRM-151, PRM-167, PEG-FGF21, BMS-986020, FG-3019, MN-001, IW001, SAR156597, GSK2126458, PAT-1251 and PBI-4050.

A further aspect of the present invention concerns combination therapy involving administering a compound of formula (I) of the present invention together with an anti-cardiovascular compound different from the compound of formula (I) to a mammal in need thereof.

A still further aspect of the present invention concerns combination therapy involving administering a compound of formula (I) in combination with a further conventional cancer treatment such as chemotherapy and/or radiotherapy, and/or treatment with immunostimulating substances, and/or gene therapy, and/or treatment with antibodies and/or treatment using dendritic cells, to a mammal in need thereof.

In an embodiment the compound of formula (I) is administered together with at least one additional therapeutic agent selected from an antineoplastic chemotherapy agent. In a further embodiment, the antineoplastic chemotherapeutic agent is selected from: all-trans retinoic acid, Actimide, Azacitidine, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Etoposide, Fludarabine, Fluorouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine. In one embodiment, a chemotherapeutic agent for use in the combination of the present agent may, itself, be a combination of different chemotherapeutic agents. Suitable combinations include FOLFOX and IFL. FOLFOX is a combination which includes 5-fluorouracil (5-FU), leucovorin, and oxaliplatin. IFL treatment includes irinotecan, 5-FU, and leucovorin.

In a further embodiment of the present invention, the further conventional cancer treatment includes radiation therapy. In some embodiments, radiation therapy includes localized radiation therapy delivered to the tumor. In some embodiments, radiation therapy includes total body irradiation.

In other embodiments of the present invention the further cancer treatment is selected from the group of immunostimulating substances e.g. cytokines and antibodies. Such cytokines may be selected from the group consisting of, but not limited to: GM-CSF, type I IFN, interleukin 21, interleukin 2, interleukin 12 and interleukin 15. The antibody is preferably an immunostimulating antibody such as anti-CD40 or anti-CTLA-4 antibodies. The immunostimulatory substance may also be a substance capable of depletion of immune inhibitory cells (e.g. regulatory T-cells) or factors, said substance may for example be E3 ubiquitin ligases. E3 ubiquitin ligases (the HECT, RING and U-box proteins) have emerged as key molecular regulators of immune cell function, and each may be involved in the regulation of immune responses during infection by targeting specific inhibitory molecules for proteolytic destruction. Several HECT and RING E3 proteins have now also been linked to the induction and maintenance of immune self-tolerance: c-Cbl, Cbl-b, GRAIL, Itch and Nedd4 each negatively regulate T cell growth factor production and proliferation.

In some embodiments of the present invention the compound of formula (I) is administered together with at least one additional therapeutic agent selected from a checkpoint inhibitor. In some embodiments of the invention, the checkpoint inhibitor is acting on one or more of the following, non-limiting group of targets: CEACAM1, galectin-9, TIM3, CD80, CTLA4, PD-1, PD-L1, HVEM, BTLA, CD160, VISTA, B7-H4, B7-2, CD155, CD226, TIGIT, CD96, LAG3, GITF, OX40, CD137, CD40, IDO, and TDO. These are known targets and some of these targets are described in Melero et al., Nature Reviews Cancer (2015). Examples of check point inhibitors administered together with the compound of formula (1) are Anti-PD-1: Nivolumab, Pembrolizumab, Cemiplimab. Anti-PD-L1: Atezolizumab, Avelumab, Durvalumab and one Anti-CTLA-4: Ipilimumab. Each one of these check point inhibitors can be made the subject of an embodiment in combination with any one of the compounds of formula (1).

In some embodiments of the present invention the compound of formula (I) is administered together with at least one additional therapeutic agent selected from an inhibitor of indoleamine-2,3-dioxygenase (IDO).

In some embodiments of the present invention the compound of formula (I) is administered together with at least one additional therapeutic agent selected from one or more inhibitors of the CTLA4 pathway. In some embodiments, the inhibitor of the CTLA4 pathway is selected from one or more antibodies against CTLA4.

In some embodiments of the present invention the compound of formula (I) is administered together with at least one additional therapeutic agent selected from one or more inhibitors of the PD-1/PD-L pathway. In some embodiments, the one or more inhibitors of the PD-1/PD-L pathway are selected from one or more antibodies or antibody fragments against PD-1, PD-L1, and/or PD-L2, or other ways by which an anti-PD1 antibodies can be induced such as mRNA based introduction of genetic material which sets forth in-body production of anti-PD1 or anti-PDL1 antibodies or fragments of such antibodies.

In a still further aspect the present invention relates to a process of preparing a compound of formula II or a pharmaceutically acceptable salt or solvate thereof comprising the step a1 where A¹, B¹ and R¹ are defined as above under formula 1;

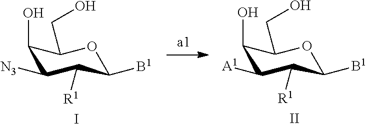

a1) Reacting the compound of formula I with a compound of formula Het¹-CC—H or Het¹-CC-TMS or Het¹-CC-TIPS in an inert solvent, such as DMF or acetonitrile, using a base, such as diisopropylethylamine or L-ascorbic acid sodium salt, catalyzed by a copper salt such as CuI or copper(II) sulfate, optionally using a reagent such as CsF or TBAF to provide a compound of the formula II.

In a still further aspect the present invention relates to a process of preparing a compound of formula IV or a pharmaceutically acceptable salt or solvate thereof comprising the step a2 where A¹, B¹ and R¹ are defined as above under formula 1;

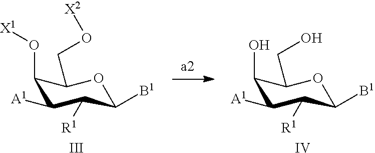

a2) Reacting a compound of formula III wherein X¹ and X² together form a protective group such as benzylidene in the presence of an acid, such as TFA or HCl, in an inert solvent, such as DCM, followed by neutralisation with a base, such as triethylamine, optionally at temperatures below room temperature, to give a compound of formula IV.

In a still further aspect the present invention relates to a process of preparing a compound of formula VI or a pharmaceutically acceptable salt or solvate thereof comprising the step a3 where B and R¹ are defined as above under formula 1;

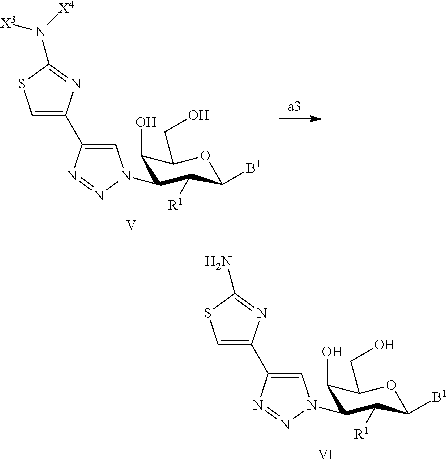

a3) Reacting a compound of formula V wherein X³ and X⁴ are protective groups such as boc-groups in the presence of an acid, such as TFA in an inert solvent, such as DCM, followed by neutralisation with a base, such as triethylamine, optionally at temperatures below room temperature, to give a compound of formula VI.

In a still further aspect the present invention relates to a process of preparing a compound of formula VIII or a pharmaceutically acceptable salt or solvate thereof comprising the step a4 where A¹, R¹ and R²⁸ are defined as above under formula 1;

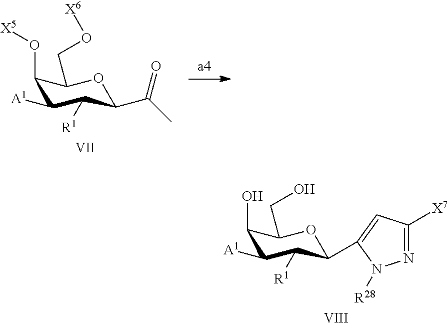

a4) Reacting a compound of formula VII wherein X⁵ and X⁶ together form a protective group such as benzylidene with either N,N-dimethylformamide dimethyl acetal or N,N-dimethylacetamide dimethyl acetal at elevated temperature followed by removal of solvents. The residues could be further reacted with R²⁸—NHNH₂ in a solvent such as ethanol in the presence of acid such as HCl to give a product of formula VIII wherein X⁷ is either a hydrogen or a methyl.

In a still further aspect the present invention relates to a process of preparing a compound of formula X or a pharmaceutically acceptable salt or solvate thereof comprising the step a5 where A¹, R¹ and R²⁸ are defined as above under formula 1;

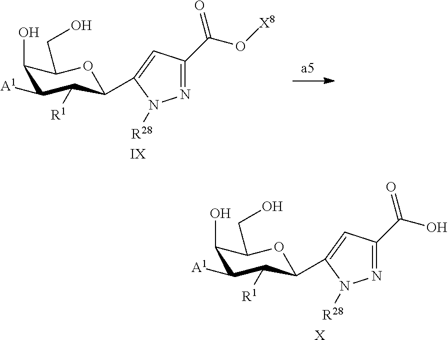

a5) Reacting a compound of formula IX wherein X⁸ is an alkyl such as ethyl with a base such as sodium hydroxide in a solvent mixture such as methanol/water/THF to give a compound of formula X.

In a still further aspect the present invention relates to a process of preparing a compound of formula XII or a pharmaceutically acceptable salt or solvate thereof comprising the step a6 where A¹, R¹ and R²⁸ are defined as above under formula 1;

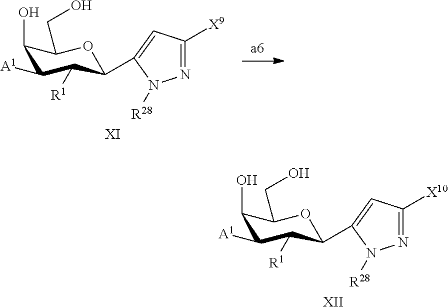

a6) Reacting a compound of the formula XI wherein X⁹ is an alkenyl group with hydrogen in the presence of a suitable catalyst such as platinum(IV) oxide in an inert solvent such as THF to give a compound of formula XII wherein X¹⁰ is an alkyl group.

In a still further aspect the present invention relates to a process of preparing a compound of formula XIX or a pharmaceutically acceptable salt or solvate thereof comprising the steps a7-a12 where A¹ is defined as above under formula 1;

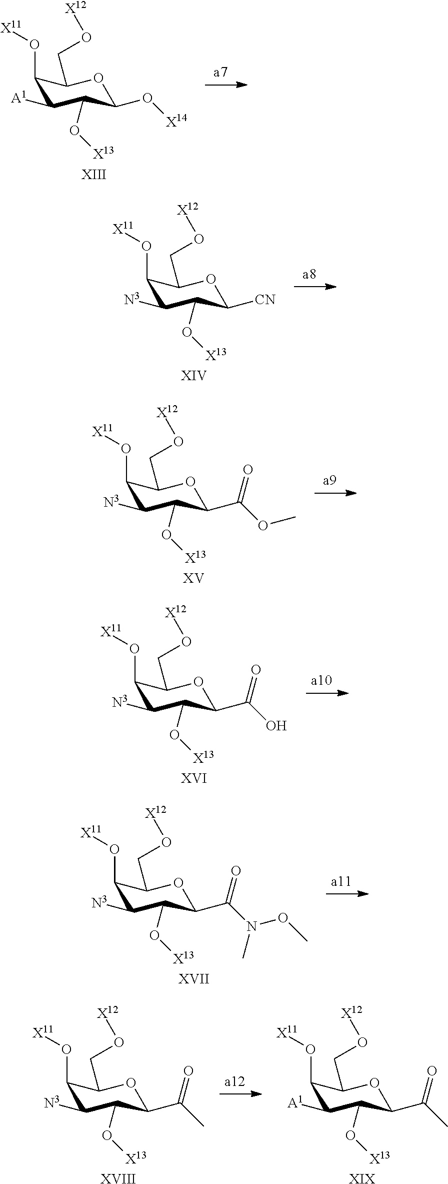

a7) Reacting a compound of the formula XIII wherein X¹¹-X¹⁴ is a protective group such as acetate, with a cyanide reagent such as trimethylsilyl cyanide in the presence of a reagent such as boron trifluoride diethyl etherate in an inert solvent such as nitromethane at 0° C. to give a compound of formula XIV.
a8) Reacting a compound of the formula XIV wherein X¹¹-X¹³ is a protective group such as acetate with acetyl chloride in methanol giving a product which is further reacted with benzaldehyde dimethylacetal in the presence of D(+)-10-camphorsulfonic acid to give a compound of formula XV, wherein X¹¹ and X¹² together form a protective group such as benzylidene and X¹³ is a hydrogen. Optionally the compound of formula XV wherein X¹¹ and X¹² together form a protective group such as benzylidene and X¹³ is a hydrogen could be reacted further with an alkyl halide such as iodomethane in the presence of a base such as cesium carbonate in a solvent such as DMF to give a compound of formula XV wherein X¹¹ and X¹² together form a protective group such as benzylidene and X¹³ is an alkyl group such as methyl.
a9) Reacting a compound of the formula XV with a base such as lithium hydroxide in a mixture of solvents such as water/THF to give a compound of formula XVI.
a10) Reacting a compound of the formula XVI with N, O-dimethylhydroxylamine in the presence of a coupling reagent such as HATU and an organic base such as DIPEA in a solvent such as DMF to give a compound of formula XVII. Optionally the compound of formula XVII wherein X¹³ is a hydrogen could be reacted further with an alkyl halide such as iodomethane in the presence of a base such as cesium carbonate in a solvent such as DMF to give a compound of formula XVII wherein X¹³ is an alkyl group such as methyl. Optionally the compound of formula XVII wherein X¹³ is a hydrogen could be reacted further with bromo(methoxy)methane in the presence of silver(I) oxide and sodium iodide in a solvent such as DMF to give a compound of formula XVII wherein X¹³ is a MOM group.
a11) Reacting a compound of formula XVII wherein X¹³ is defined as above with methylmagnesium bromide in an inert solvent such as THF to give a compound of formula XVIII.
a12) Reacting a compound of formula XVIII wherein X¹³ is defined as above with a compound of formula Het¹-CC—H or Het¹-CC-TMS in an inert solvent, such as DMF or acetonitrile, using a base, such as DIPEA or L-ascorbic acid sodium salt, catalyzed by a copper salt such as CuI or copper(II) sulfate, optionally using a reagent such as CsF to provide a compound of formula XIX.

In a still further aspect the present invention relates to a process of preparing a compound of formula XXII or a pharmaceutically acceptable salt or solvate thereof comprising the step a13 where R¹ and R²⁸ are defined as above under formula 1;

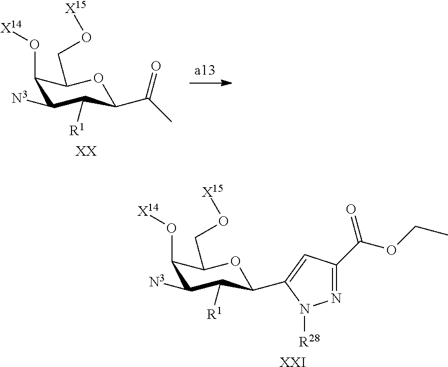

a13) Reacting a compound of formula XX wherein X and X together form a protective group such as benzylidene with diethyl oxalate using a base, such as lithium diisopropylamide in an inert solvent, such as THF at temperatures ranging from −78° C. to room temperature followed by removal of solvents. The residues could be further reacted with R²⁸—NHNH₂ in a solvent such as ethanol in the presence of acid such as acetic acid to give a product of formula XXI wherein X¹⁴ and X¹⁵ either together form a protective group such as benzylidene or are hydrogens.

In a still further aspect the present invention relates to a process of preparing a compound of formula XXIII or a pharmaceutically acceptable salt or solvate thereof comprising the step a14 where A¹, R¹ and R²⁸ are defined as above under formula 1;

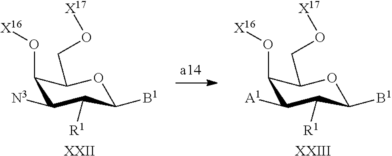

a14) Reacting a compound of formula XXII wherein X¹⁶ and X¹⁷ together form a protective group such as benzylidene with a compound of formula Het¹-CC—H or Het¹-CC-TMS in an inert solvent, such as DMF or acetonitrile, using a base, such as DIPEA or L-ascorbic acid sodium salt, catalyzed by a copper salt such as CuI or copper(II) sulfate, optionally using a reagent such as CsF to provide a compound of formula XXIII.

In a still further aspect the present invention relates to a process of preparing a compound of formula XXVI or a pharmaceutically acceptable salt or solvate thereof comprising the steps a15-a16 where A¹, R¹ and R²⁸ are defined as above under formula 1;

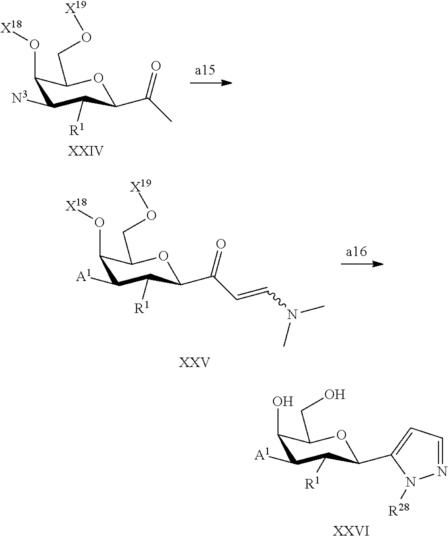

a15) Reacting a compound of formula XXIV wherein X¹⁸ and X¹⁹ together form a protective group such as benzylidene with N,N-dimethylformamide dimethyl acetal at elevated temperature to give a compound of formula XXV.
a16) Reacting a compound of formula XXV with R²⁸—NHNH₂ in a solvent such as ethanol in the presence of acid such as HCl to give a compound of formula XXVI.

In a still further aspect the present invention relates to a process of preparing a compound of formula XXXI or a pharmaceutically acceptable salt or solvate thereof comprising the steps a17-a20 where R¹ and R²⁸ are defined as above under formula 1_

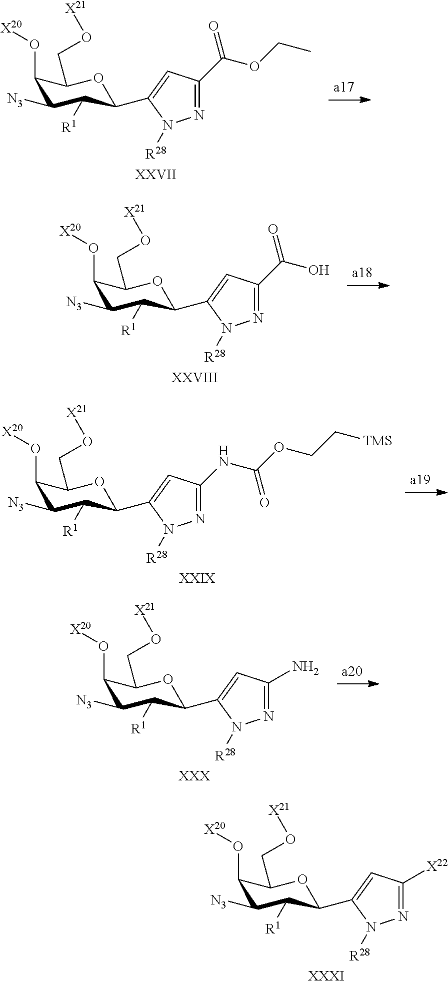

a17) Reacting a compound of formula XXVII wherein X²⁰ and X²¹ together form a protective group such as benzylidene with a base such as sodium hydroxide in a solvent mixture such as methanol/water/THF to give a compound of formula XXVIII.
a18) Reacting a compound of formula XXVIII with diphenylphosphoryl azide and 2-trimethylsilylethanol using a base such as triethylamine in an inert solvent such as 1,4-dioxane to give a compound of formula XXIX.
a19) Reacting a compound of formula XXIX with TBAF in an inert solvent such as THF to give a compound of formula XXX.
a20) Reacting a compound of formula XXX with a cupper salt, such as CuCl or CuBr and a reagent, such as pentyl nitrite, in the presence of a reagent, such as LiCl or CuBr₂, at 0° C. to give a compound of formula XXXI wherein X²² is a halide such as chlorine or bromine.

In a still further aspect the present invention relates to a process of preparing a compound of formula XXXI or a pharmaceutically acceptable salt or solvate thereof comprising the step a21 where A¹, R¹ and R²⁸ are defined as above under formula 1;

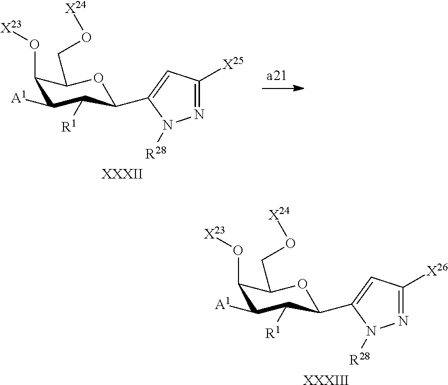

a21) Reacting a compound of formula XXXII wherein X²³ and X²⁴ together form a protective group such as benzylidene and X²⁵ is a halide such as bromine with a compound of the formula L¹-X²⁶, wherein L¹ is defined as a boronic acid, borinatester, tinalkyl or zincalkyl suitable for cross-coupling reactions such as Suzuki, Stille or Negishi couplings in the presence of a catalyst such as Pd(PPh₃)₄ or Pd(dppf)Cl₂ in a suitable solvent such as 1,4-dioxane/water optionally in the presence of a base such as K₂CO₃, optionally at elevated temperature to give a compound of formula XXXIII wherein X²⁶ is an alkenyl group.

In a still further aspect the present invention relates to a process of preparing a compound of formula XXXVI or a pharmaceutically acceptable salt or solvate thereof comprising the steps a22-a23 where R¹ and R²⁸ are defined as above under formula 1;

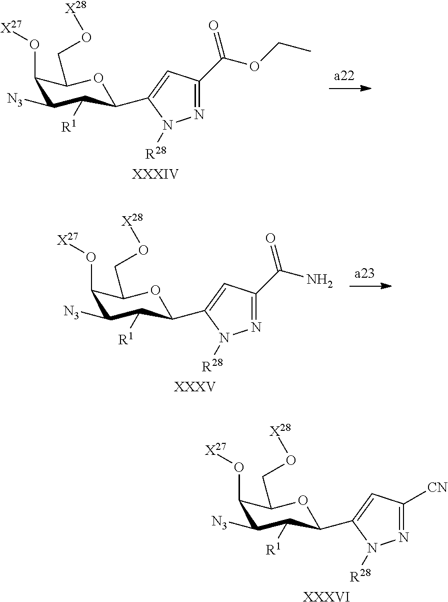

a22) Reacting a compound of formula XXXII wherein X²⁷ and X²⁸ together form a protective group such as benzylidene with ammonia in a suitable solvent such as methanol optionally at elevated temperature to give a compound of formula XXXV.
a23) Reacting a compound of formula XXXV with pyridine and trifluoroacetic anhydride in an inert solvent such as THF to give a compound of formula XXXVI.

In a still further aspect the present invention relates to a process of preparing a compound of formula XXXIX or a pharmaceutically acceptable salt or solvate thereof comprising the steps a24-a25 where R²⁸ is defined as above under formula 1;

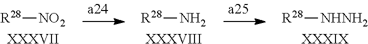

a24) Reacting a compound of the formula XXXVII with acetic acid in the presence of iron at elevated temperature to give a compound of formula XXXVIII.
a25) Reacting a compound of the formula XXXVIII with sodium nitrite in the presence of HCl and acetic acid in water solution to give a product that is reacted with tin(II)chloride to give a compound of formula XXXIX.

In a still further aspect the present invention relates to a process of preparing a compound of formula XXXXI or a pharmaceutically acceptable salt or solvate thereof comprising the step a26 where R²⁸ is defined as above under formula 1;

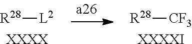

a26) Reacting a compound of formula XXXX wherein L is a leaving group such as a halide such as bromine with methyl 2,2-difluoro-2-fluorosulfonylacetate in the presence of a copper salt such as CuI in an inert solvent such as DMF optionally at elevated temperature to give a compound of formula XXXXI.

In a still further aspect the present invention relates to a process of preparing a compound of formula Het¹-CC—H or Het¹-CC-TMS comprising the step a27 wherein Het¹ is defined as above under formula 1;

a27) Reacting a compound of formula Het¹-L³ wherein L³ is defined as a leaving group such as chlorine or bromine with trimethylsilaneacetylene using a palladium catalyst such as bis(triphenylphosphine)palladium(II)-chloride, CuI and a base such as DIPEA in an inert solvent, such as THF, to give a compound of formula Het¹-CC—H or Het¹-CC-TMS.

In a still further aspect the present invention relates to a process of preparing a compound of formula XXXXIV comprising the step a28 wherein R^{1a}, R^{2a} and R^{3a} are as defined under formula 11 of Het¹ which is defined as above under formula 1;

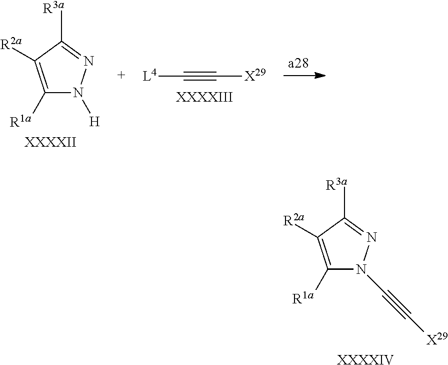

a28) Reacting a compound of formula XXXXII with a compound of formula XXXXIII wherein L⁴ is defined as a halide such as bromine or iodine and X²⁹ is either a hydrogen or a protective group such as triisopropylsilane in the presence of CuI and a base such as Cs₂CO₃ in an inert solvent, such as 1,4-diaxane and PEG400, to give a compound of formula XXXXIV.

### DETAILED DESCRIPTION OF THE INVENTION

The present compounds of formula (1) differ from prior art compounds particularly in that the pyranose ring is β-D-galactopyranose. It is important to emphasize that alpha and beta anomers are very different isomers and it is by no means considered to be obvious to the skilled person to expect same or similar activity of both anomers. Consequently, alpha and beta anomers do not in general posses the same activity, and this is common knowledge to the skilled person. The compounds of the present invention are novel β-D-galactopyranose compounds that unexpectedly have high affinity galectin-1 and/or 3 inhibitors.

In a broad aspect the present invention concerns a β-D-galactopyranose compound of formula (1)

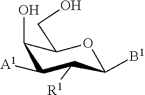

wherein
the pyranose ring is β-D-galactopyranose, and
A¹, B¹ and R¹ are as defined above.

In an embodiment Het¹ is a five membered heteroaromatic ring selected from the group consisting of formulas 2, 3, 4, 5, 9, 10 and 11, wherein the asterix * indicates the carbon atom of the heteroaromatic ring that is covalently attached to the triazole group in formula A¹:

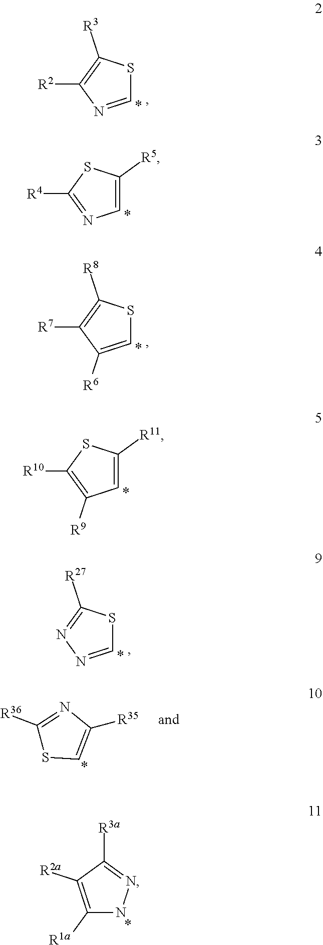

wherein R^{1a}, R^{2a}, R^{3a}, R² to R¹¹, R²⁷, R³⁵ and R³⁶ are independently selected from H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl.

In another embodiment Het¹ is a six membered heteroaromatic ring selected from the group consisting of formulas 6, 7 and 8, wherein the asterix * indicates the carbon atom of the heteroaromatic ring that is covalently attached to the triazole group in formula A¹:

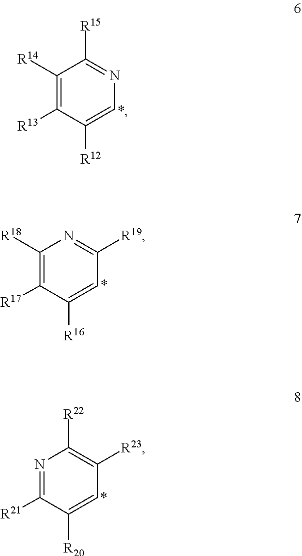

wherein R¹² to R²³ are independently selected from H; halogen; OH; CN; SH; S—C₁₋₆ alkyl; C₁₋₆ alkyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl.

In a further embodiment Het1 is

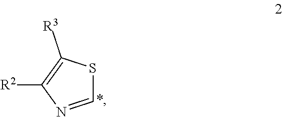

wherein R² is selected from the group consisting of hydrogen, methyl, OH and halogen;

R³ is selected from the group consisting of hydrogen, C₁₋₆ alkyl and halogen.

In a still further embodiment Het1 is formula 2 wherein R² is selected from the group consisting of hydrogen, methyl, OH, F and Cl; and R³ is selected from the group consisting of hydrogen, methyl, F and Cl.

In a further embodiment Het1 is formula 2 wherein R² is selected from the group consisting of hydrogen, C₁₋₃ alkyl, e.g. methyl, and halogen, e.g. Cl; and R³ is selected from the group consisting of hydrogen, C₁₋₃ alkyl, e.g. methyl, and halogen, e.g. Cl. In one embodiment both R² and R³ are hydrogen. In another embodiment one of R² and R³ is hydrogen and the other is selected from the group consisting of hydrogen, C₁₋₃ alkyl, and halogen.

In a further embodiment Het1 is

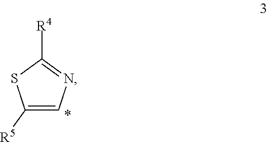

wherein
R⁴ is selected from the group consisting of OH, C₁₋₆ alkyl, halogen and amino;
R⁵ is selected from the group consisting of hydrogen, C₁₋₆ alkyl and halogen.

In a further embodiment Het1 is formula 3 wherein
R⁴ is selected from the group consisting of OH and amino;
R⁵ is hydrogen.

In a further embodiment Het1 is

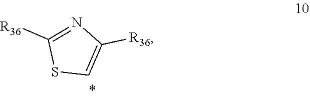

wherein
R³⁵ and R³⁶ are independently selected from hydrogen, C₁₋₆ alkyl, amino and halogen.

In a further embodiment Het1 is

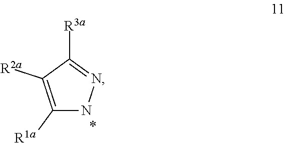

wherein R^{1a} is selected from the group consisting of hydrogen, OH, C₁₋₆ alkyl, amino and halogen;
R^{2a} is selected from the group consisting of hydrogen, OH, C₁₋₆ alkyl, amino and halogen; and
R^{3a} is selected from the group consisting of hydrogen, OH, C₁₋₆ alkyl, amino and halogen.

In a further embodiment Het1 is formula 11 wherein R^{1a} is hydrogen; R^{2a} is hydrogen; and R^{3a} is selected from the group consisting of halogen, such as Cl.

In a further embodiment B¹ is a pyrazol substituted with one to three groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R²⁸—C₁₋₆ alkyl, c) C₃₋₆ cycloalkyl optionally substituted with one or more of C₁₋₆ alkyl, amino, CN, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, i) C₂-alkynyl, and j) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R³³)(R³⁴)N; or b) (C₁₋₆ alkyl-SO₂)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;

wherein
R²⁹ is hydrogen or C₁₋₆ alkyl;
R₃₀ is hydrogen or C₁₋₆ alkyl; or
(R²⁹)(R³⁰)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R³¹ is hydrogen, C₁₋₆ alkyl, C₁₋₆ alkylcarbonyl, or C₁₋₆ alkylsulfonyl;
R³² is hydrogen or C₁₋₆ alkyl; or
(R³¹)(R³²)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R³³ is hydrogen, C₁₋₆ alkyl, C₁₋₆ alkylcarbonyl, or C₁₋₆ alkylsulfonyl;
R³⁴ is hydrogen or C₁₋₆ alkyl; or
(R³³)(R³⁴)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl; In a still further embodiment B¹ is a pyrazolyl substituted with one, two or three groups selected from the group consisting of pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R²⁸-C₁₋₆ alkyl, c) C₃₋₆ cycloalkyl optionally substituted with one or more of C₁₋₆ alkyl, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, and j) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R³³)(R³⁴)N; or b) (C₁₋₆ alkyl-S02)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R²⁹ is hydrogen or C₁₋₆ alkyl;
R₃₀ is hydrogen or C₁₋₆ alkyl; or
(R²⁹)(R³⁰)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R³¹ is hydrogen, C₁₋₆ alkyl, C₁₋₆ alkylcarbonyl, or C₁₋₆ alkylsulfonyl;
R³² is hydrogen or C₁₋₆ alkyl; or
(R³¹)(R³²)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R³³ is hydrogen, C₁₋₆ alkyl, C₁₋₆ alkylcarbonyl, or C₁₋₆ alkylsulfonyl;
R³⁴ is hydrogen or C₁₋₆ alkyl; or
(R³³)(R³⁴)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl.

In a further embodiment B¹ is a pyrazolyl substituted with a phenyl optionally substituted with a group selected from methyl, CF₃, and halogen.

In a still further embodiment B¹ is

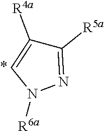

wherein the asterix * indicates the carbon atom of the pyrazol ring that is covalently attached to the galactopyranose,
wherein R^{4a} is selected from the group consisting of hydrogen, pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R²⁸-C₁₋₆ alkyl, c) C₃₋₆ cycloalkyl optionally substituted with one or more of C₁₋₆ alkyl, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, and j) R²⁸; wherein R²⁸, R²⁹, R³⁰, R³¹ and R³² are as defined above,
R^{5a} is selected from the group consisting of hydrogen, pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R²⁸-C₁₋₆ alkyl, c) C₃₋₆ cycloalkyl optionally substituted with one or more of C₁₋₆ alkyl, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, and j) R²⁸; wherein R²⁸, R²⁹, R³⁰, R³¹ and R³² are as defined above,
R^{6a} is selected from the group consisting of pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R²⁸-C₁₋₆ alkyl, c) C₃₋₆ cycloalkyl optionally substituted with one or more of C₁₋₆ alkyl, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, and j) R²⁸; wherein R²⁸, R²⁹, R³⁰, R³¹ and R³² are as defined above. Typically, R^{4a} is hydrogen, R^{5a} is selected from the group consisting of hydrogen, halogen, cyano, C₁₋₄ alkyl, C₂₋₄ alkenyl, COOC₁₋₄ alkyl, and COOH and R^{6a} is selected from the group consisting of a phenyl substituted with a group selected from C₁₋₄ alkyl, such as methyl, C₁₋₃ alkyl substituted with one or more halogen, such as F, e.g. CF₃ and halogen, such as Br, F, Cl; a pyridinyl substituted with a group selected from C₁₋₄ alkyl, such as methyl, C₁₋₃ alkyl substituted with one or more halogen, such as F, e.g. CF₃ and halogen, such as Br, Cl; and benzothiazolyl substituted with a group selected from C₁₋₄ alkyl, such as methyl. Typically, R^{4a} is hydrogen, R^{5a} is hydrogen and R^{6a} is a phenyl substituted with a group selected from methyl, CF₃ and Cl. Typically, R^{4a} is hydrogen, R^{5a} is selected from the group consisting of hydrogen, Cl, Br, cyano, methyl, ethyl, isopropyl, ethenyl, methylethenyl, COOCH₃, and COOH and R^{6a} is a phenyl substituted with a group selected from methyl, CF₃, Br, F, and Cl. Typically, R^{4a} is hydrogen, R^{5a} is hydrogen and R^{6a} is a pyridinyl substituted with a group selected from CF₃, Br, and Cl.

Typically, R^{4a} is hydrogen, R^{5a} is hydrogen and R^{6a} is benzothiazolyl substituted with a methyl.

In a further embodiment R¹ is a) OC₁₋₆ alkyl optionally substituted with one or more halogen, phenyl, phenyl substituted with one or more groups selected form OH and halogen, CN, OR³⁷, NR³⁸R³⁹, and CONH₂, wherein R³⁷ is selected from the group consisting of H, CN, a halogen, methyl optionally substituted with a F, OCH₃ optionally substituted with a F, OCH₂CH₃ optionally substituted with a F, OH, and R⁴⁰—CONH— wherein R⁴⁰ is selected from C₁₋₃ alkyl and cyclopropyl, R³⁸ is selected from the group consisting of H, CN, a halogen, methyl optionally substituted with a F, OCH₃ optionally substituted with a F, OCH₂CH₃ optionally substituted with a F, OH, and R⁴¹—CONH— wherein R⁴¹ is selected from C₁₋₃ alkyl and cyclopropyl, and R³⁹ is selected from the group consisting of H, CN, a halogen, methyl optionally substituted with a F, OCH₃ optionally substituted with a F, OCH₂CH₃ optionally substituted with a F, OH, and R⁴²—CONH— wherein R⁴² is selected from C₁₋₃ alkyl and cyclopropyl,

In a still further embodiment R¹ is OC₁₋₆ alkyl optionally substituted with one or two halogen, phenyl, phenyl substituted with one or more groups selected form OH and halogen, CN, OR³⁷, NR³⁸R³⁹, and CONH₂, wherein R³⁷ is selected from the group consisting of H, CN, a halogen, methyl optionally substituted with a F, OCH₃ optionally substituted with a F, OCH₂CH₃ optionally substituted with a F, OH, and R⁴⁰—CONH— wherein R⁴⁰ is selected from C₁₋₃ alkyl and cyclopropyl, R³⁸ is selected from the group consisting of H, CN, a halogen, methyl optionally substituted with a F, OCH₃ optionally substituted with a F, OCH₂CH₃ optionally substituted with a F, OH, and R⁴¹—CONH— wherein R⁴¹ is selected from C₁₋₃ alkyl and cyclopropyl, and R³⁹ is selected from the group consisting of H, CN, a halogen, methyl optionally substituted with a F, OCH₃ optionally substituted with a F, OCH₂CH₃ optionally substituted with a F, OH, and R⁴²—CONH— wherein R⁴² is selected from C₁₋₃ alkyl and cyclopropyl. Typically, R¹ is selected from OC₁₋₆ alkyl optionally substituted with one or more halogen, such as R¹ is selected from OC₁₋₃ alkyl, e.g. methoxy.

In a further embodiment R¹ is b) branched OC₃₋₆ alkyl optionally substituted with one or more halogen, CN, OR⁴³, NR⁴⁴R⁴⁵, and CONH₂, wherein R⁴³ is selected from the group consisting of H, CN, a halogen, methyl optionally substituted with a F, OCH₃ optionally substituted with a F, OCH₂CH₃ optionally substituted with a F, OH, and R⁴⁶—CONH— wherein R⁴⁶ is selected from C₁₋₃ alkyl and cyclopropyl, R⁴⁴ is selected from the group consisting of H, CN, a halogen, methyl optionally substituted with a F, OCH₃ optionally substituted with a F, OCH₂CH₃ optionally substituted with a F, OH, and R⁴⁷—CONH— wherein R⁴⁷ is selected from C₁₋₃ alkyl and cyclopropyl, and R⁴⁵ is selected from the group consisting of H, CN, a halogen, methyl optionally substituted with a F, OCH₃ optionally substituted with a F, OCH₂CH₃ optionally substituted with a F, OH, and R⁴⁸—CONH— wherein R⁴⁸ is selected from C₁₋₃ alkyl and cyclopropyl.

In a still further embodiment R¹ is c) cyclic OC₃₋₆ alkyl optionally substituted with one or more halogen, CN, OR⁴⁹, NR⁵⁰R⁵¹, and CONH₂, wherein R⁴⁹ is selected from the group consisting of H, CN, a halogen, methyl optionally substituted with a F, OCH₃ optionally substituted with a F, OCH₂CH₃ optionally substituted with a F, OH, and R⁵²—CONH— wherein R⁵² is selected from C₁₋₃ alkyl and cyclopropyl, R⁵⁰ is selected from the group consisting of H, CN, a halogen, methyl optionally substituted with a F, OCH₃ optionally substituted with a F, OCH₂CH₃ optionally substituted with a F, OH, and R⁵³—CONH— wherein R⁵³ is selected from C₁₋₃ alkyl and cyclopropyl, and R⁵¹ is selected from the group consisting of H, CN, a halogen, methyl optionally substituted with a F, OCH₃ optionally substituted with a F, OCH₂CH₃ optionally substituted with a F, OH, and R⁵⁴—CONH— wherein R⁵⁴ is selected from C₁₋₃ alkyl and cyclopropyl.

Preferably, the compound of formula (1) selected from any one of the compounds of examples 1-39; or a pharmaceutically acceptable salt or solvate thereof.

The skilled person will understand that it may be necessary to adjust or change the order of steps in the processes a1-a28, and such change of order is encompassed by the aspects of the process as described above in the reaction schemes and accompanying description of the process steps.

Furthermore, the skilled person will understand that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldipheylsilyl or trimethylsilyl), AcO(acetoxy), TBS(t-butyldimethylsilyl), TMS(trimethylsilyl), PMB (p-methoxybenzyl), and tetrahydropyranyl. Suitable protecting groups for carboxylic acid include (C₁₋₆)-alkyl or benzyl esters. Suitable protecting groups for amino include t-butyloxycarbonyl, benzyloxycarbonyl, 2-(trimethylsilyl)-ethoxy-methyl or 2-trimethylsilylethoxycarbonyl (Teoc). Suitable protecting groups for S include S—C(═N)NH₂, TIPS.

The protection and deprotection of functional groups may take place before or after any reaction in the above-mentioned processes.

Furthermore the skilled person will appreciate, that, in order to obtain compounds of the invention in an alternative, and on some occasions more convenient manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

In a still further embodiment the compound (1) is on free form. “On free form” as used herein means a compound of formula (1), either an acid form or base form, or as a neutral compound, depending on the substitutents. The free form does not have any acid salt or base salt in addition. In one embodiment the free form is an anhydrate. In another embodiment the free form is a solvate, such as a hydrate.

In a further embodiment the compound of formula (1) is a crystalline form. The skilled person may carry out tests in order to find polymorphs, and such polymorphs are intended to be encompassed by the term “crystalline form” as used herein.

Whenever a “compound of formula (1)” is used herein it means the compound of formula (1) in any form incl the free form or as a salt thereof, such as a pharmaceutically acceptable salt thereof, unless otherwise indicated herein or clearly contradicted by context.

When the compounds and pharmaceutical compositions herein disclosed are used for the above treatment, a therapeutically effective amount of at least one compound is administered to a mammal in need of said treatment.

The term “C₁₋ₓ alkyl” as used herein means an alkyl group containing 1-x carbon atoms, e.g. C₁₋₅ or C₁₋₆, such as methyl, ethyl, propyl, butyl, pentyl or hexyl.

The term “branched C₃₋₆ alkyl” as used herein means a branched alkyl group containing 3-6 carbon atoms, such as isopropyl, isobutyl, tert-butyl, isopentyl, 3-methylbutyl, 2,2-dimethylpropyl, n-hexyl, 2-methylpentyl, 2,2-dimethylbutyl, 2,3-dimethylbutyl.

The term “C₃₋ₓ cycloalkyl” as used herein means a cyclic alkyl group containing 3-x carbon atoms, e.g. C₃₋₆ or C₃₋₇, such as cyclopropyl, cyclobutyl, cyclopentyl, cyclohexyl, cycloheptyl, and 1-methylcyclopropyl.

The term “C₅₋₇ cycloalkyl” as used herein means a cyclic alkyl group containing 5-7 carbon atoms, such as cyclopentyl, cyclohexyl, or cycloheptyl.

The term “Oxo” as used herein means an oxygen atom with double bonds, also indicated as ═O.

The term “CN” as used herein means a nitril (interchangeable with cyano).

The term “halogen” as used herein means Cl, F, Br or I.

The term “halo-C₁₋₆ alkyl” as used herein means one or more halogens linked to a C₁₋₆ alkyl, such as CF₃, CH(Cl)CHF₂.

The term “C₁₋₆ alkoxy” as used herein means an oxygen linked to a C₁₋₆ alkyl, such as methoxy or ethoxy.

The term “C₁₋₆ alkylthio” as used herein means a sulphur linked to a C₁₋₆ alkyl, such as thiomethoxy or thioethoxy.

The term “halo-C₁₋₆ alkoxy” as used herein means one or more halogens linked to a C₁₋₆ alkoxy, such as CH(F₂)CH(Br)O—.

The term “C₁₋₆ alkoxycarbonyl” as used herein means a C₁₋₆ alkoxy linked to a carbonyl, such as methoxycarbonyl (CH₂OC(═O)).

The term “a five or six membered heteroaromatic ring” as used herein means one five membered heteroaromatic ring or one six membered heteroaromatic ring. The five membered heteroaromatic ring contains 5 ring atoms of which one to four are heteroatoms selected from N, O, and S. The six membered heteroaromatic ring contains 6 ring atoms of which one to five are heteroatoms selected from N, O and S. Examples include thiophene, furan, pyran, pyrrole, imidazole, pyrazole, isothiazole, isooxazole, pyridine, pyrazine, pyrimidine and pyridazine. When such heteroaromatic rings are substituents they are termed thiophenyl, furanyl, pyranyl, pyrrolyl, imidazolyl, pyrazolyl, isothiazolyl, isooxazolyl, pyridinyl, pyrazinyl, pyrimidinyl and pyridazinyl. Also included are oxazoyl, thiazoyl, thiadiazoly, oxadiazoyl, and pyridonyl.

The term “a heterocycle, such as heteroaryl or heterocycloalkyl” as used herein means a heterocycle consisting of one or more 3-7 membered ring systems containing one or more heteroatoms and wherein such ring systems may optionally be aromatic. The term “a heteroaryl” as used herein means a mono or bicyclic aromatic ringsystem containing one or more heteroatoms, such as 1-10, e.g. 1-6, selected from O, S, and N, including but not limited to oxazolyl, oxadiazolyl, thiophenyl, thiadiazolyl, thiazolyl, pyridyl, pyrimidinyl, pyridonyl, pyrimidonyl, quinolinyl, azaquionolyl, isoquinolinyl, azaisoquinolyl, quinazolinyl, azaquinazolinyl, bensozazoyl, azabensoxazoyl, bensothiazoyl, or azabensothiazoyl. The term “a heterocycloalkyl” as used herein means a mono or bicyclic 3-7 membered alifatic heterocycle containing one or more heteroatoms, such as 1-7, e.g. 1-5, selected from O, S, and N, including but not limited to piperidinyl, tetrahydropyranyl, tetrahydrothipyranyl, or piperidonyl.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The treatment may either be performed in an acute or in a chronic way. The patient to be treated is preferably a mammal; in particular, a human being, but it may also include animals, such as dogs, cats, cows, sheep and pigs.

The term “a therapeutically effective amount” of a compound of formula (1) of the present invention as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In a still further aspect, the present invention relates to a pharmaceutical composition comprising the compound of formula (1) and optionally a pharmaceutically acceptable additive, such as a carrier or an excipient.

As used herein “pharmaceutically acceptable additive” is intended without limitation to include carriers, excipients, diluents, adjuvant, colorings, aroma, preservatives etc. that the skilled person would consider using when formulating a compound of the present invention in order to make a pharmaceutical composition.

The adjuvants, diluents, excipients and/or carriers that may be used in the composition of the invention must be pharmaceutically acceptable in the sense of being compatible with the compound of formula (1) and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person skilled within the art.

As mentioned above, the compositions and particularly pharmaceutical compositions as herein disclosed may, in addition to the compounds herein disclosed, further comprise at least one pharmaceutically acceptable adjuvant, diluent, excipient and/or carrier. In some embodiments, the pharmaceutical compositions comprise from 1 to 99% by weight of said at least one pharmaceutically acceptable adjuvant, diluent, excipient and/or carrier and from 1 to 99% by weight of a compound as herein disclosed. The combined amount of the active ingredient and of the pharmaceutically acceptable adjuvant, diluent, excipient and/or carrier may not constitute more than 100% by weight of the composition, particularly the pharmaceutical composition.

In some embodiments, only one compound as herein disclosed is used for the purposes discussed above.

In some embodiments, two or more of the compounds as herein disclosed are used in combination for the purposes discussed above.

The composition, particularly pharmaceutical composition comprising a compound set forth herein may be adapted for oral, intravenous, topical, intraperitoneal, nasal, buccal, sublingual, or subcutaneous administration, or for administration via the respiratory tract in the form of, for example, an aerosol or an air-suspended fine powder. Therefore, the pharmaceutical composition may be in the form of, for example, tablets, capsules, powders, nanoparticles, crystals, amorphous substances, solutions, transdermal patches or suppositories.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The term “and/or” as used herein is intended to mean both alternatives as well as each of the alternatives individually. For instance, the expression “xxx and/or yyy” means “xxx and yyy”; “xxx”; or “yyy”, all three alternatives are subject to individual embodiments.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The present invention is further illustrated by the following examples that, however, are not to be construed as limiting the scope of protection. The features disclosed in the foregoing description and in the following examples may, both separately and in any combination thereof, be material for realizing the invention indiverse forms thereof.

### Experimental Procedures (Evaluation of Kd Values)

The affinity of Example 1-39 for galectins were determined by a fluorescence anisotropy assay where the compound was used as an inhibitor of the interaction between galectin and a fluorescein tagged saccharide probe as described Sarme, P., Kahl-Knutsson, B., Huflejt, M., Nilsson, U. J., and Leffler H. (2004) Fluorescence polarization as an analytical tool to evaluate galectin-ligand interactions. Anal. Biochem. 334: 36-47, (Sarme et al., 2004) and Monovalent interactions of Galectin-1 By Salomonsson, Emma; Larumbe, Amaia; Tejler, Johan; Tullberg, Erik; Rydberg, Hanna; Sundin, Anders; Khabut, Areej; Frejd, Torbjorn; Lobsanov, Yuri D.; Rini, James M.; et al, From Biochemistry (2010), 49(44), 9518-9532, (Salomonsson et al., 2010).

| | | | Gal-1 | Gal-3 |
|---|---|---|---|---|
| | | | Kd | Kd |
| Example | Name | Structure | (μM) | (μM) |
| 1 | 1-{5-{3-[4-(4- Chlorothiazol-2-yl)-1H- 1,2,3-triazol-1-yl]-3- deoxy-2-O-methyl-β-D- galactopyranosyl}-1H- 1,2-pyrazol-1-yl}-5- chloro-2- (trifluoromethyl)benzene | 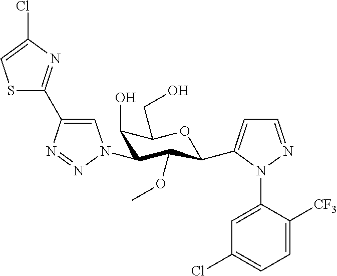 | 0.036 | 0.45 |
| 2 | 5-Chloro-1-{5-{3-deoxy- 2-O-methyl-3-[4-(2- thiazolyl)-1H-1,2,3- triazol-1-yl]-β-D- galactopyranosyl}-1H- 1,2-pyrazol-1-yl}-2- (trifluoromethyl)benzene | 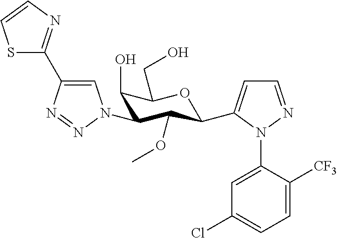 | 0.17 | 7.56 |
| 3 | 5-Chloro-1-{5-{3-deoxy- 3-[4-(5-methylthiazol-2- yl)-1H-1,2,3-triazol-1-yl]- 2-O-methyl-β-D- galactopyranosyl}-1H- 1,2-pyrazol-1-yl}-2- (trifluoromethyl)benzene | 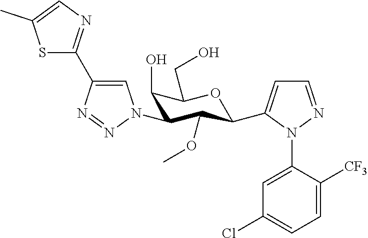 | 0.47 | 0.44 |
| 4 | 5-Chloro-1-{5-{3-deoxy- 3-[4-(4-methylthiazol-2- yl)-1H-1,2,3-triazol-1-yl]- 2-O-methyl-β-D- galactopyranosyl}-1H- 1,2-pyrazol-1-yl}-2- (trifluoromethyl)benzene | 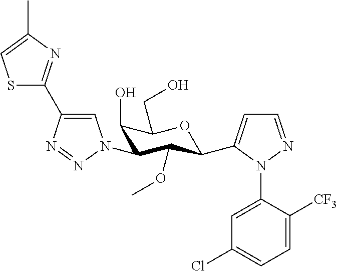 | 0.26 | 3.26 |
| 5 | 5-Chloro-1-{5-{3-deoxy- 3-[4-(2-thiazolyl)-1H- 1,2,3-triazol-1-yl]-β-D- galactopyranosyl}-1H- 1,2-pyrazol-1-yl}-2- (trifluoromethyl)benzene | 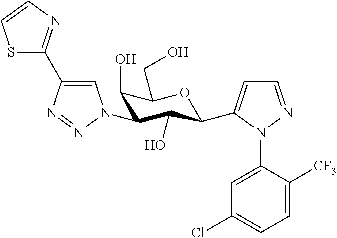 | 0.21 | 4.83 |
| 6 | 1-{5-{3-[4-(2- Aminothiazol-4-yl)-1H- 1,2,3-triazol-1-yl]-3- deoxy-β-D- galactopyranosyl}-1H- 1,2-pyrazol-1-yl}-5- chloro-2- (trifluoromethyl)benzene | 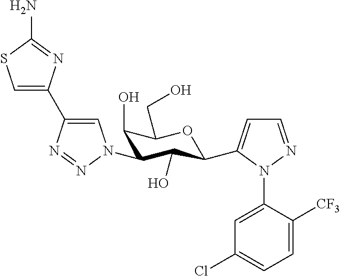 | 0.12 | 10.8 |
| 7 | 5-Chloro-1-{5-{3-deoxy- 3-[4-(2-hydroxythiazol-4- yl)-1H-1,2,3-triazol-1-yl]- β-D-galactopyranosyl}- 1H-1,2-pyrazol-1-yl}-2- (trifluoromethyl)benzene | 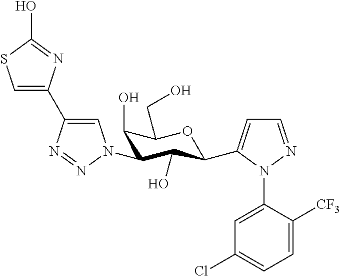 | 0.029 | 5.83 |
| 8 | 1-{5-{3-[4-(3-Chloro-1H- 1,2-pyrazol-1-yl)-1H- 1,2,3-triazol-1-yl]-3- deoxy-β-D- galactopyranosyl}-1H- 1,2-pyrazol-1-yl}-5- chloro-2- (trifluoromethyl)benzene | 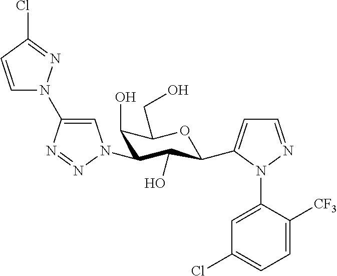 | 0.27 | 0.88 |
| 9 | 1-{5-{3-[4-(4- Chlorothiazol-2-yl)-1H- 1,2,3-triazol-1-yl]-3- deoxy-β-D- galactopyranosyl}-3- methyl-1H-1,2-pyrazol-1- yl}-5-chloro-2- (trifluoromethyl)benzene | 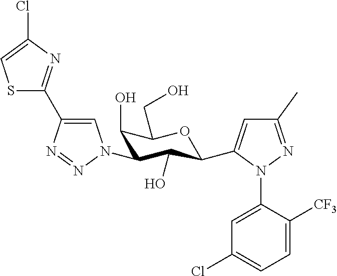 | 0.037 | 0.37 |
| 10 | 1-{5-{3-[4-(5- Chlorothiazol-2-yl)-1H- 1,2,3-triazol-1-yl]-3- deoxy-β-D- galactopyranosyl}-1H- 1,2-pyrazol-1-yl}-5- chloro-2- (trifluoromethyl)benzene | 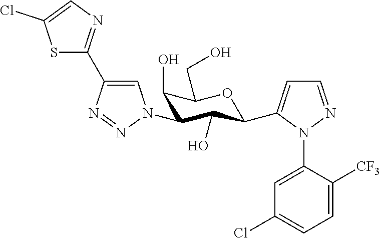 | 0.043 | 0.33 |
| 11 | 1-{5-{3-[4-(4- Chlorothiazol-2-yl)-1H- 1,2,3-triazol-1-yl]-3- deoxy-β-D- galactopyranosyl}-1H- 1,2-pyrazol-1-yl}-5- chloro-2- (trifluoromethyl)benzene | 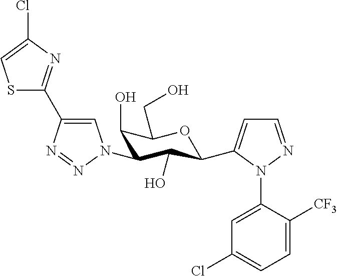 | 0.048 | 0.43 |
| 12 | 1-{5-{3-[4-(5- Chlorothiazol-2-yl)-1H- 1,2,3-triazol-1-yl]-3- deoxy-2-O-methyl-β-D- galactopyranosyl}-1H- 1,2-pyrazol-1-yl}-5- chloro-2- (trifluoromethyl)benzene | 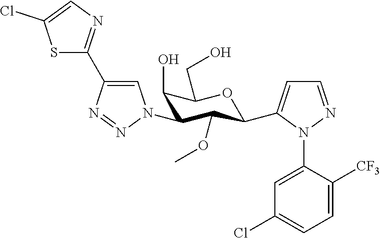 | 0.024 | 0.39 |
| 13 | 1-{5-{3-[4-(4- Chlorothiazol-2-yl)-1H- 1,2,3-triazol-1-yl]-3- deoxy-2-O-methyl-β-D- galactopyranosyl}-3- methyl-1H-1,2-pyrazol-1- yl}-5-chloro-2- (trifluoromethyl)benzene | 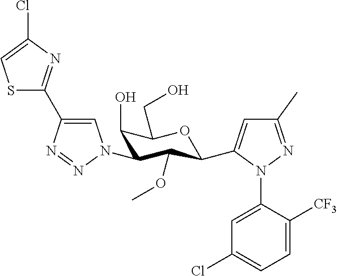 | 0.036 | 0.29 |
| 14 | 5-Chloro-1-{5-{3-deoxy- 2-O-methyl-3-[4-(2- thiazolyl)-1H-1,2,3- triazol-1-yl]-β-D- galactopyranosyl}-3- methyl-1H-1,2-pyrazol-1- yl}-2- (trifluoromethyl)benzene | 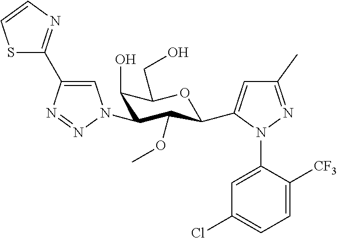 | 0.15 | 5.2 |
| 15 | 1-{5-{3-[4-(4- Chlorothiazol-2-yl)-1H- 1,2,3-triazol-1-yl]-3- deoxy-2-O-methyl-β-D- galactopyranosyl}-3- methyl-1H-1,2-pyrazol-1- y1}-2- methylbenzothiazole | 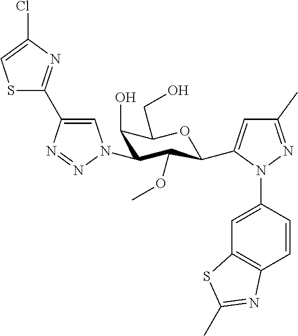 | 0.067 | 2.0 |
| 16 | 1-{5-{3-[4-(4- Chlorothiazol-2-yl)-1H- 1,2,3-triazol-1-yl]-3- deoxy-2-O-methyl-β-D- galactopyranosyl}-1H- 1,2-pyrazol-1-yl}-2- methylbenzothiazole | 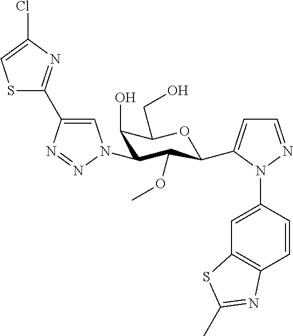 | 0.038 | 1.5 |
| 17 | 1-{5-{3-[4-(4- Chlorothiazol-2-yl)-1H- 1,2,3-triazol-1-yl]-3- deoxy-2-O-methyl-β-D- galactopyranosyl}-3- ethoxycarbonyl-1H-1,2- pyrazol-1-yl}-5-chloro-2- (trifluoromethyl)benzene | 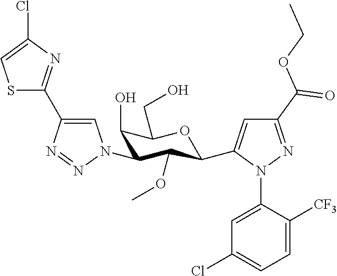 | 0.24 | 0.88 |
| 18 | 1-{3-Carboxy-5-{3-[4-(4- chlorothiazol-2-yl)-1H- 1,2,3-triazol-1-yl]-3- deoxy-2-O-methyl-β-D- galactopyranosyl}-1H- 1,2-pyrazol-1-yl}-5- chloro-2- (trifluoromethyl)benzene | 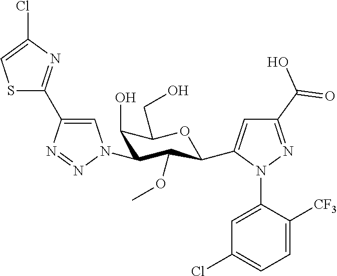 | 0.42 | 0.32 |
| 19 | 5-Bromo-1-{5-{3-[4-(4- chlorothiazol-2-yl)-1H- 1,2,3-triazol-1-yl]-3- deoxy-β-D- galactopyranosyl}-1H- 1,2-pyrazol-1-yl}-2- (trifluoromethyl)benzene | 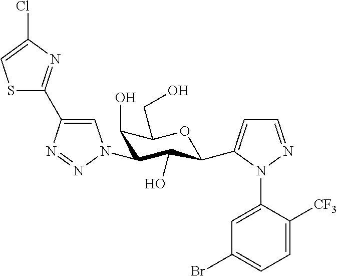 | 0.043 | 0.28 |
| 20 | 5-Bromo-1-{5-{3-[4-(4- chlorothiazol-2-yl)-1H- 1,2,3-triazol-1-yl]-3- deoxy-2-O-methyl-β-D- galactopyranosyl}-1H- 1,2-pyrazol-1-yl}-2- (trifluoromethyl)benzene | 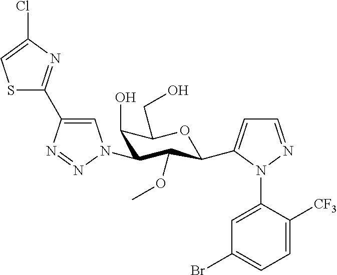 | 0.031 | 0.41 |
| 21 | 1-{5-{3-[4-(4- Chlorothiazol-2-yl)-1H- 1,2,3-triazol-1-yl]-3- deoxy-2-O-methyl-β-D- galactopyranosyl}-1H- 1,2-pyrazol-1-yl}-4,5- dichloro-2- (trifluoromethyl)benzene | 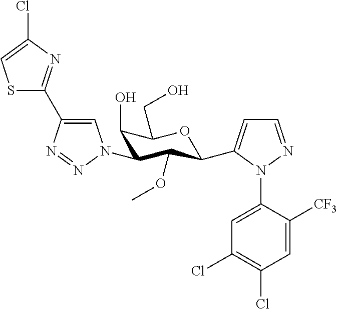 | 0.035 | 1.1 |
| 22 | 1-{5-{3-[4-(4- Chlorothiazol-2-yl)-1H- 1,2,3-triazol-1-yl]-3- deoxy-2-O-methyl-β-D- galactopyranosyl}-1H- 1,2-pyrazol-1-yl}-5- chloro-4-fluoro-2- (trifluoromethyl)benzene | 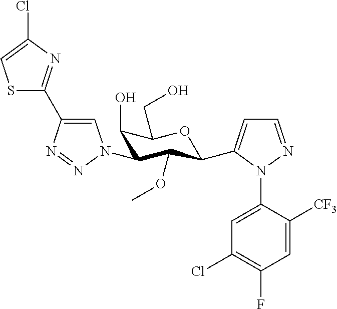 | 0.043 | 0.85 |
| 23 | 5-Bromo-1-{5-{3-[4-(4- chlorothiazol-2-yl)-1H- 1,2,3-triazol-1-yl]-3- deoxy-2-O-methyl-β-D- galactopyranosyl}-1H- 1,2-pyrazol-1-yl}-4- fluoro-2- (trifluoromethyl)benzene | 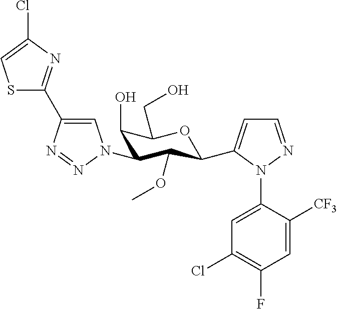 | 0.024 | 0.29 |
| 24 | 5-Bromo-1-{5-{3-[4-(4- chlorothiazol-2-yl)-1H- 1,2,3-triazol-1-yl]-3- deoxy-β-D- galactopyranosyl}-1H- 1,2-pyrazol-1-yl}-4- fluoro-2- (trifluoromethyl)benzene | 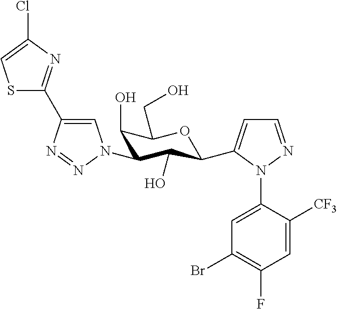 | 0.036 | 0.20 |
| 25 | 1-{5-{3-[4-(4- Chlorothiazol-2-yl)-1H- 1,2,3-triazol-1-yl]-3- deoxy-2-O-methyl-β-D- galactopyranosyl}-3- chloro-1H-1,2-pyrazol-1- yl}-5-chloro-2- (trifluoromethyl)benzene | 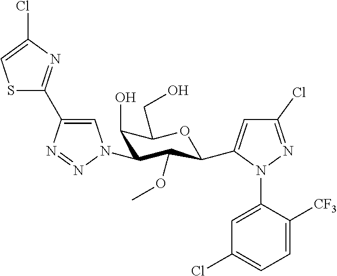 | 0.040 | 0.18 |
| 26 | 1-{3-Bromo-5-{3-[4-(4- chlorothiazol-2-yl)-1H- 1,2,3-triazol-1-yl]-3- deoxy-2-O-methyl-β-D- galactopyranosyl}-1H- 1,2-pyrazol-1-yl}-5- chloro-2- (trifluoromethyl)benzene | 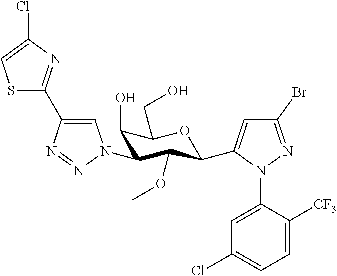 | 0.066 | 0.75 |
| 27 | 1-{5-{3-[4-(4- Chlorothiazol-2-yl)-1H- 1,2,3-triazol-1-yl]-3- deoxy-2-O-methyl-β-D- galactopyranosyl}-3-(1- methylethenyl)-1H-1,2- pyrazol-1-yl}-5-chloro-2- (trifluoromethyl)benzene | 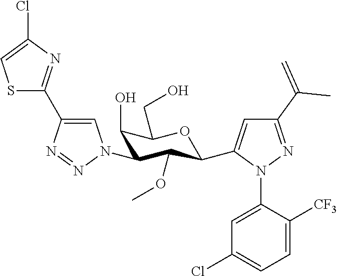 | 0.54 | 1.2 |
| 28 | 1-{5-{3-[4-(4- Chlorothiazol-2-yl)-1H- 1,2,3-triazol-1-yl]-3- deoxy-2-O-methyl-β-D- galactopyranosyl}-3- isopropyl-1H-1,2- pyrazol-1-yl}-5-chloro-2- (trifluoromethyl)benzene | 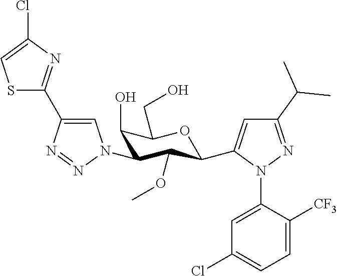 | 0.18 | 0.32 |
| 29 | 1-{5-{3-[4-(4- Chlorothiazol-2-yl)-1H- 1,2,3-triazol-1-yl]-3- deoxy-2-O-methyl-β-D- galactopyranosyl}-3- ethenyl-1H-1,2-pyrazol- 1-yl}-5-chloro-2- (trifluoromethyl)benzene | 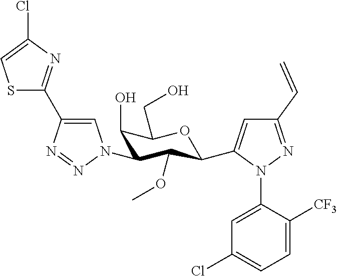 | 0.19 | 0.48 |
| 30 | 1-{5-{3-[4-(4- Chlorothiazol-2-yl)-1H- 1,2,3-triazol-1-yl]-3- deoxy-2-O-methyl-β-D- galactopyranosyl}-3- ethyl-1H-1,2-pyrazol-1- yl}-5-chloro-2- (trifluoromethyl)benzene | 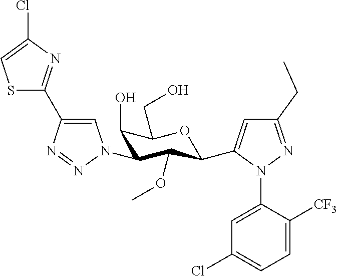 | 0.10 | 0.68 |
| 31 | 1-{5-{3-[4-(4- Chlorothiazol-2-yl)-1H- 1,2,3-triazol-1-yl]-3- deoxy-2-O-methyl-β-D- galactopyranosyl}-3- cyano-1H-1,2-pyrazol-1- yl}-5-chloro-2- (trifluoromethyl)benzene | 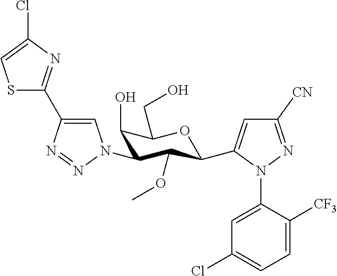 | 0.071 | 0.4 |
| 32 | 3-{5-{3-[4-(4- Chlorothiazol-2-yl)-1H- 1,2,3-triazol-1-yl]-3- deoxy-2-O-methyl-β-D- galactopyranosyl}-1H- 1,2-pyrazol-1-yl}-5- chloro-2- (trifluoromethyl)pyridine | 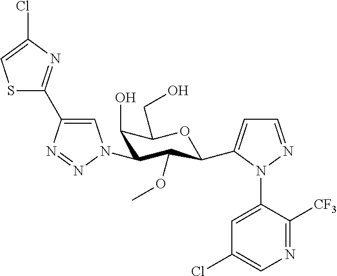 | 0.050 | 0.65 |
| 33 | 5-Bromo-3-{5-{3-[4-(4- chlorothiazol-2-yl)-1H- 1,2,3-triazol-1-yl]-3- deoxy-2-O-methyl-β-D- galactopyranosyl}-1H- 1,2-pyrazol-1-yl}-2- (trifluoromethyl)pyridine | 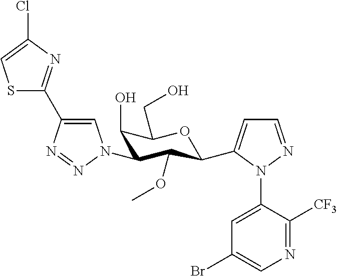 | 0.032 | 0.53 |
| 34 | 1-{5-{3-[4-(2- Aminothiazol-4-yl)-1H- 1,2,3-triazol-1-yl]-3- deoxy-2-O-methyl-β-D- galactopyranosyl}-1H- 1,2-pyrazol-1-yl}-5- chloro-2- (trifluoromethyl)benzene | 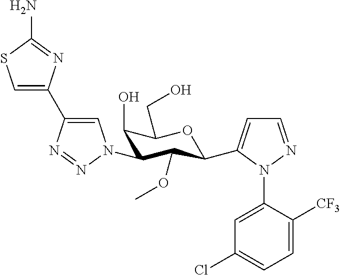 | 0.049 | 6.7 |
| 35 | 5-Chloro-1-{5-{3-deoxy- 3-[4-(2-hydroxythiazol-4- yl)-1H-1,2,3-triazol-1-yl]- 2-O-methyl-β-D- galactopyranosyl}-1H- 1,2-pyrazol-1-yl}-2- (trifluoromethyl)benzene | 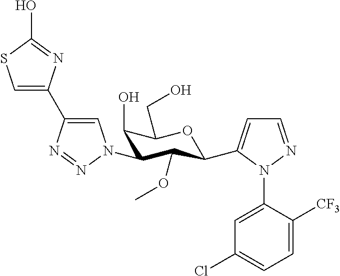 | 0.031 | 4.2 |
| 36 | 4,5-Dichloro-1-{5-{3- deoxy-3-[4-(2- hydroxythiazol-4-yl)-1H- 1,2,3-triazol-1-yl]-2-O- methyl-β-D- galactopyranosyl}-1H- 1,2-pyrazol-1-yl}-2- (trifluoromethyl)benzene | 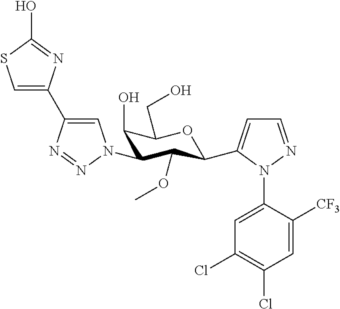 | 0.026 | 9.4 |
| 37 | 5-Bromo-1-{5-{3-deoxy- 3-[4-(2-hydroxythiazol-4- yl)-1H-1,2,3-triazol-1-yl]- 2-O-methyl-β-D- galactopyranosyl}-1H- 1,2-pyrazol-1-yl}-4- fluoro-2- (trifluoromethyl)benzene | 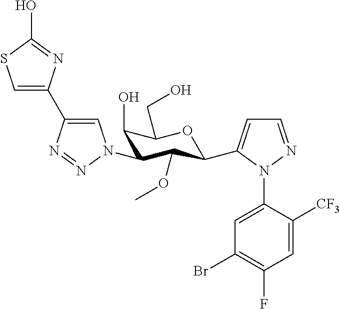 | 0.017 | 4.7 |
| 38 | 5-Chloro-1-{5-{3-deoxy- 2-O-methyl-3-[4-(4- thiazolyl)-1H-1,2,3- triazol-1-yl]-β-D- galactopyranosyl}-1H- 1,2-pyrazol-1-yl}-2- (trifluoromethyl)benzene | 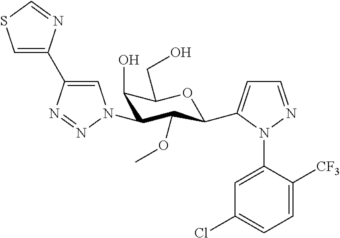 | 0.12 | 4.9 |
| 39 | 1-{3-Aminocarbonyl-5- {3-[4-(4-chlorothiazol-2- yl)-1H-1,2,3-triazol-1-yl]- 3-deoxy-2-O-methyl-β-D- galactopyranosyl}-1H- 1,2-pyrazol-1-yl}-5- chloro-2- (trifluoromethyl)benzene | 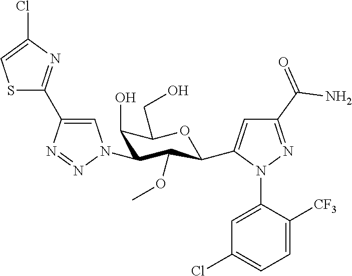 | 0.20 | 0.32 |

### Synthesis of Examples and Intermediates

### General Experimental

Nuclear Magnetic Resonance (NMR) spectra were recorded on a 400 MHz Bruker AVANCE III 500 instrument or a Varian instrument at 400 MHz, at 25° C. Chemical shifts are reported in ppm (d) using the residual solvent as internal standard. Peak multiplicities are expressed as follow: s, singlet; d, doublet; dd, doublet of doublets; t, triplet; dt, doublet of triplet; q, quartet; m, multiplet; br s, broad singlet. LC-MS were acquired on an Agilent 1200 HPLC coupled with an Agilent MSD mass spectrometer operating in ES (+) ionization mode. Column: XBridge C18 (4.6×50 mm, 3.5 μm) or SunFire C18 (4.6×50 mm, 3.5 μm). Solvent A water+0.1% TFA and solvent B Acetonitrile+0.1% TFA or solvent A water (10 mM Ammonium hydrogen carbonate) and solvent B Acetonitrile. Wavelength: 254 nM. Alternatively, LC-MS were acquired on an Agilent 1100 HPLC coupled with an Agilent MSD mass spectrometer operating in ES (+) ionization mode. Column: Waters symmetry 2.1×30 mm C18 or Chromolith RP-18 2×50 mm. Solvent A water+0.1% TFA and solvent B Acetonitrile+0.1% TFA. Wavelength 254 nm.

Preparative HPLC was performed on a Gilson 215. Flow: 25 mL/min Column: XBrige prep C18 10 μm OBD (19×250 mm) column. Wavelength: 254 nM. Solvent A water (10 mM Ammonium hydrogen carbonate) and solvent B Acetonitrile. Alternatively, preparative HPLC were acquired on a Gilson system. Flow: 15 ml/min Column: kromasil 100-5-C18 column. Wavelength: 220 nm. Solvent A water+0.1% TFA and solvent B Acetonitrile+0.1% TFA.

The following abbreviations are used
aq: aqueous
Calcd: Calculated
MeCN: Acetonitrile
CuI: Copper Iodide
DCM: Dichloromethane
DIPEA: Diisopropylethylamine
DMF: N,N-dimethylformamide
ESI-MS: Electrospray ionization mass spectrometry
EtOAc or EA: Ethylacetate
Et₃N: Triethylamine
h: hour(s)
HATU: 1-[Bis(dimethylamino)methylene]-1H-1,2,3-triazolo[4,5-b]pyridinium 3-oxid hexafluorophosphate
HPLC: High performance liquid chromatography
LC: Liquid Chromatography
MeCN: Acetonitrile
mL: milliliter
MeOH: Methanol
MeOD: Deuterated methanol
mm: millimeter
mM: millimolar
MS: Mass spectroscopy
nm: nanometer
NaOMe: Sodium methoxide
N₂: Nitrogen gas
NMR: Nuclear magnetic resonance
PE: petroleum ether
pH: acidity
Prep: Preparative
rt: Room temperature
TFA: trifluoroacetic acid
THF: Tetrahydrofuran
TMS: Trimethylsilyl
UV: Ultraviolet
Å: Ångström

### Synthesis of Example 1-39 from their Respective Intermediates 1-39

When naming examples and intermediates with aryl groups connected directly to C1 of the galactose unit the following methodology has been used. The highest priority has been given to the aryl furthest away from the galactose C1, regardless of IUPAC rules.

### Example 1

### 1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene

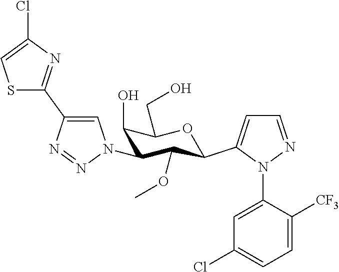

To a solution of 1-[5-(3-azido-3-deoxy-2-O-methyl-β-D-galactopyranosyl)-1H-1,2-pyrazol-1-yl]-5-chloro-2-(trifluoromethyl)benzene (62 mg, 0.14 mmol) in DMF (2 mL) 2-(4-chlorothiazol-2-yl)ethynyltrimethylsilane (59.8 mg, 0.28 mmol), copper(II) sulfate pentahydrate (34.6 mg, 0.14 mmol) and (+)-sodium L-ascorbate (27.4 mg, 0.14 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 m/min, UV 254) to give the title compound (45.2 mg, 55%). ESI-MS m/z calcd for [C₂₂H₁₉Cl₂F₃N₆O₄S] [M+H]⁺: 591.1; found: 591.0 ¹H NMR (400 MHz, Methanol-d₄) δ 8.76 (s, 1H), 7.94 (d, J=8.4 Hz, 1H), 7.82 (d, J=8.4 Hz, 1H), 7.77-7.76 (m, 2H), 7.47 (s, 1H), 6.88 (d, J=2.0 Hz, 1H), 4.94 (dd, J=10.4, 2.8 Hz, 1H), 4.71-4.30 (m, 1H), 4.19 (d, J=9.2 Hz, 1H), 4.08 (d, J=2.4 Hz, 1H), 3.74-3.57 (m, 3H), 2.95 (s, 3H).

### Example 2

### 5-Chloro-1-{5-{3-deoxy-2-O-methyl-3-[4-(2-thiazolyl)-1H-1,2,3-triazol-1-yl]-β-D-galactopyranosyl}-1H-1,2-pyrazol-1-yl}-2-(trifluoromethyl)benzene

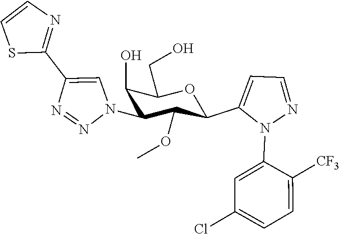

To a solution of 1-[5-(3-azido-3-deoxy-2-O-methyl-β-D-galactopyranosyl)-1H-1,2-pyrazol-1-yl]-5-chloro-2-(trifluoromethyl)benzene (62 mg, 0.14 mmol) in DMF (2 mL) trimethyl(2-thiazol-2-ylethynyl)silane (50.2 mg, 0.28 mmol), copper(II) sulfate pentahydrate (34.6 mg, 0.14 mmol) and (+)-sodium L-ascorbate (27.4 mg, 0.14 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 m/min, UV 254) to give the title compound (39.5 mg, 51%). ESI-MS m/z calcd for [C₂₂H₂₀ClF₃N₆O₄S] [M+H]⁺: 557.1; found: 557.2. ¹H NMR (400 MHz, Methanol-d₄) δ 8.73 (s, 1H), 7.94 (d, J=8.4 Hz, 1H), 7.90 (d, J=3.6 Hz, 1H), 7.87-7.68 (m, 3H), 7.64 (d, J=3.2 Hz, 1H), 6.88 (d, J=2.0 Hz, 1H), 4.94 (dd, J=10.4, 2.8 Hz, 1H), 4.72-4.26 (m, 1H), 4.20 (d, J=9.2 Hz, 1H), 4.09 (d, J=2.4 Hz, 1H), 3.84-3.55 (m, 3H), 2.95 (s, 3H).

### Example 3

### 5-Chloro-1-{5-{3-deoxy-3-[4-(5-methylthiazol-2-yl)-1H-1,2,3-triazol-1-yl]-2-O-methyl-β-D-galactopyranosyl}-1H-1,2-pyrazol-1-yl}-2-(trifluoromethyl)benzene

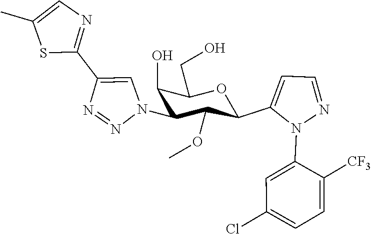

To a solution of 1-[5-(3-azido-3-deoxy-2-O-methyl-β-D-galactopyranosyl)-1H-1,2-pyrazol-1-yl]-5-chloro-2-(trifluoromethyl)benzene (70 mg, 0.16 mmol) in DMF (3 mL) trimethyl-[2-(5-methylthiazol-2-yl)ethynyl]silane (61.1 mg, 0.31 mmol), copper(II) sulfate pentahydrate (39.0 mg, 0.16 mmol) and (+)-sodium L-ascorbate (31.0 mg, 0.16 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 m/min, UV 254) to give the title compound (49.3 mg, 55%). ESI-MS m/z calcd for [C₂₃H₂₂ClF₃N₆O₄S] [M+H]⁺: 571.1; found: 571.0. ¹H NMR (400 MHz, Methanol-d₄) δ 8.57 (s, 1H), 7.84 (d, J=8.4 Hz, 1H), 7.72 (d, J=8.4 Hz, 1H), 7.67 (d, J=1.6 Hz, 1H), 7.60-7.47 (m, 2H), 6.78 (d, J=2.0 Hz, 1H), 4.83 (dd, J=10.4, 2.8 Hz, 1H), 4.58-4.20 (m, 1H), 4.10 (d, J=8.4 Hz, 1H), 3.98 (d, J=2.0 Hz, 1H), 3.72-3.46 (m, 3H), 2.84 (s, 3H), 2.44 (s, 3H).

### Example 4

### 5-Chloro-1-{5-{3-deoxy-3-[4-(4-methylthiazol-2-yl)-1H-1,2,3-triazol-1-yl]-2-O-methyl-β-D-galactopyranosyl}-1H-1,2-pyrazol-1-yl}-2-(trifluoromethyl)benzene

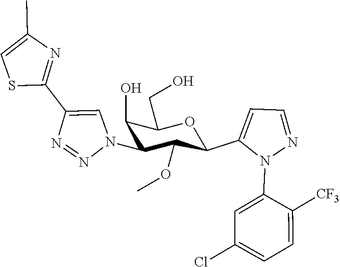

To a solution of 1-[5-(3-azido-3-deoxy-2-O-methyl-β-D-galactopyranosyl)-1H-1,2-pyrazol-1-yl]-5-chloro-2-(trifluoromethyl)benzene (45 mg, 0.10 mmol) in DMF (3 mL) trimethyl-[2-(4-methylthiazol-2-yl)ethynyl]silane (58.9 mg, 0.30 mmol), copper(II) sulfate pentahydrate (25.1 mg, 0.10 mmol) and (+)-sodium L-ascorbate (19.9 mg, 0.10 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 m/min, UV 254) to give the title compound (26.3 mg, 46%). ESI-MS m/z calcd for [C₂₃H₂₂ClF₃N₆O₄S] [M+H]⁺: 571.1; found: 571.2. ¹H NMR (400 MHz, Methanol-d₄) δ 8.61 (s, 1H), 7.84 (d, J=8.4 Hz, 1H), 7.78-7.44 (m, 3H), 7.09 (s, 1H), 6.78 (d, J=2.0 Hz, 1H), 4.84 (dd, J=10.4, 2.8 Hz, 1H), 4.57-4.19 (m, 1H), 4.10 (d, J=9.2 Hz, 1H), 3.99 (d, J=2.4 Hz, 1H), 3.67-3.47 (m, 3H), 2.85 (s, 3H), 2.38 (s, 3H).

### Example 5

### 5-Chloro-1-{5-{3-deoxy-3-[4-(2-thiazolyl)-1H-1,2,3-triazol-1-yl]-β-D-galactopyranosyl}-1H-1,2-pyrazol-1-yl}-2-(trifluoromethyl)benzene

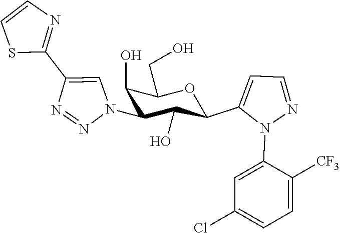

To a solution of 1-[5-(3-azido-3-deoxy-β-D-galactopyranosyl)-1H-1,2-pyrazol-1-yl]-5-chloro-2-(trifluoromethyl)benzene (40 mg, 0.092 mmol) in DMF (2 mL) trimethyl(2-thiazol-2-ylethynyl)silane (50.2 mg, 0.28 mmol), copper(II) sulfate pentahydrate (23 mg, 0.092 mmol) and (+)-sodium L-ascorbate (18.3 mg, 0.092 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 m/min, UV 254) to afford the title compound (25.7 mg, 51%). ESI-MS m/z calcd for [C₂₁H₁₈ClF₃N₆O₄S] [M+H]⁺: 543.1; found: 542.8. ¹H NMR (400 MHz, Methanol-d₄) δ 8.59 (s, 1H), 7.92 (d, J=8.4 Hz, 1H), 7.89 (d, J=3.2 Hz, 1H), 7.80 (d, J=8.4 Hz, 1H), 7.78-7.74 (m, 1H), 7.73 (d, J=1.6 Hz, 1H), 7.63 (d, J=3.2 Hz, 1H), 6.77 (d, J=1.6 Hz, 1H), 4.88-4.83 (m, 1H), 4.74-4.53 (m, 1H), 4.20 (d, J=9.2 Hz, 1H), 4.12 (d, J=2.8 Hz, 1H), 3.70-3.53 (m, 3H).

### Example 6

### 1-{5-{3-[4-(2-Aminothiazol-4-yl)-1H-1,2,3-triazol-1-yl]-3-deoxy-β-D-galactopyranosyl}-1H-1,2-pyrazol-1-yl}-5-chloro-2-(trifluoromethyl)benzene

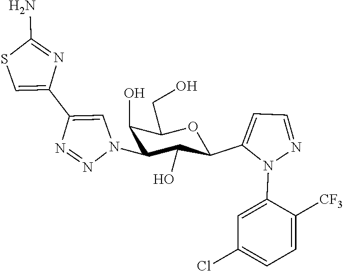

To a solution of 1-{5-{3-{4-[2-(di-tert-butoxycarbonylamino)thiazol-4-yl]-1H-1,2,3-triazol-1-yl}-3-deoxy-β-D-galactopyranosyl}-1H-1,2-pyrazol-1-yl}-5-chloro-2-(trifluoromethyl)benzene (66 mg, 0.087 mmol) in DCM (6 mL) TFA (0.20 mL) was added and the mixture was stirred overnight at rt. Et₃N (0.5 mL) was added at 0° C.

The mixture was concentrated and was dissolved in DMF (3 mL). The mixture was filtered, and the filtrate was purified by prep HPLC (MeCN/H₂O (10 mmol/L NH₄HCO₃), X-Select 10 μm 19*250 mm, 20 m/min, UV 254) to afford the title compound (29.8 mg, 61%). ESI-MS m/z calcd for [C₂₁H₁₉ClF₃N₇O₄S] [M+H]⁺: 558.1; found: 557.7. ¹H NMR (400 MHz, Methanol-d₄) δ 8.24 (s, 1H), 7.91 (d, J=8.4 Hz, 1H), 7.80 (d, J=8.4 Hz, 1H), 7.78-7.70 (m, 2H), 6.94 (s, 1H), 6.76 (d, J=2.0 Hz, 1H), 4.79 (dd, J=10.4, 2.8 Hz, 1H), 4.71-4.50 (m, 1H), 4.18 (d, J=8.4 Hz, 1H), 4.10 (d, J=2.8 Hz, 1H), 3.70-3.53 (m, 3H).

### Example 7

### 5-Chloro-1-{5-{3-deoxy-3-[4-(2-hydroxythiazol-4-yl)-1H-1,2,3-triazol-1-yl]-β-D-galactopyranosyl}-1H-1,2-pyrazol-1-yl}-2-(trifluoromethyl)benzene

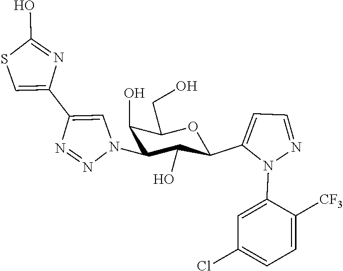

To a solution of 1-[5-(3-azido-3-deoxy-β-D-galactopyranosyl)-1H-1,2-pyrazol-1-yl]-5-chloro-2-(trifluoromethyl)benzene (60 mg, 0.14 mmol) in DMF (2 mL) 4-(2-trimethylsilylethynyl)thiazol-2-ol (54.6 mg, 0.28 mmol), copper(II) sulfate pentahydrate (34.5 mg, 0.14 mmol) and (+)-sodium L-ascorbate (27.4 mg, 0.14 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (31.3 mg, 40%). ESI-MS m/z calcd for [C₂₁H₁₈ClF₃N₆O₅S] [M+H]⁺: 559.1; found: 558.8. ¹H NMR (400 MHz, Methanol-d₄) δ 8.28 (s, 1H), 7.82 (d, J=8.8 Hz, 1H), 7.71 (d, J=8.8 Hz, 1H), 7.69-7.64 (m, 2H), 6.64 (s, 1H), 6.58 (s, 1H), 4.72 (d, J=9.2 Hz, 1H), 4.63-4.38 (m, 1H), 4.09 (d, J=7.2 Hz, 1H), 4.00 (s, 1H), 3.53 (s, 3H).

### Example 8

### 1-{5-{3-[4-(3-Chloro-1H-1,2-pyrazol-1-yl)-1H-1,2,3-triazol-1-yl]-3-deoxy-β-D-galactopyranosyl}-1H-1,2-pyrazol-1-yl}-5-chloro-2-(trifluoromethyl)benzene

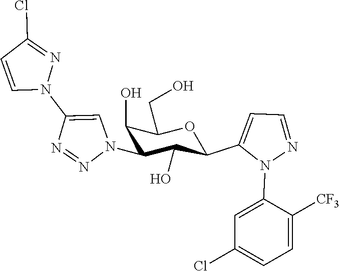

To a solution of 1-[5-(3-azido-3-deoxy-β-D-galactopyranosyl)-1H-1,2-pyrazol-1-yl]-5-chloro-2-(trifluoromethyl)benzene (60 mg, 0.14 mmol) in DMF (3 mL) 2-(3-chloropyrazol-1-yl)ethynyl(triisopropyl)silane (58.7 mg, 0.21 mmol), copper(II) sulfate pentahydrate (34.5 mg, 0.14 mmol), (+)-sodium L-ascorbate (27.4 mg, 0.14 mmol) and tetrabutylammonium fluoride (0.14 mL, 1 M in THF, 0.14 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (28.7 mg, 37%). ESI-MS m/z calcd for [C₂₁H₁₈Cl₂F₃N₇O₄] [M+H]⁺: 560.1; found: 559.8. ¹H NMR (400 MHz, Methanol-d₄) δ 8.24 (s, 1H), 8.14 (d, J=2.4 Hz, 1H), 7.82 (d, J=8.8 Hz, 1H), 7.71 (d, J=8.4 Hz, 1H), 7.68-7.52 (m, 2H), 6.67 (d, J=1.6 Hz, 1H), 6.41 (d, J=2.4 Hz, 1H), 4.71 (dd, J=10.4, 2.8 Hz, 1H), 4.63-4.43 (m, 1H), 4.09 (d, J=9.2 Hz, 1H), 4.02 (d, J=2.4 Hz, 1H), 3.58-3.47 (m, 3H).

### Example 9

### 1-{5-{3-[4-(4-Chlorothiazol-2-yl)-1H-1,2,3-triazol-1-yl]-3-deoxy-β-D-galactopyranosyl}-3-methyl-1H-1,2-pyrazol-1-yl}-5-chloro-2-(trifluoromethyl)benzene

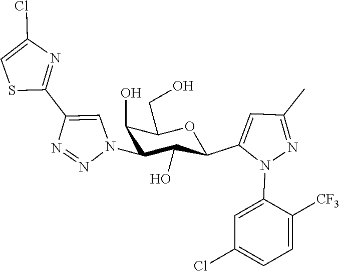

A solution of 3,7-anhydro-6,8-O-benzylidene-5-[4-(4-chlorothiazol-2-yl)-1H-1,2,3-triazol-1-yl]-5-deoxy-4-O-(methoxymethyl)-D-glycero-L-manno-2-octulose (90 mg, 0.18 mmol) in N,N-dimethylacetamide dimethyl acetal (2.0 mL) was stirred 3 h at 100° C. The mixture was concentrated to dryness and the residue was dissolved in EtOH (5.0 mL). [5-Chloro-2-(trifluoromethyl)phenyl]hydrazine (56 mg, 0.27 mmol) and concentrated HCl (0.2 mL) were added and the mixture was stirred 3 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (20.9 mg, 20%). ESI-MS m/z calcd for [C₂₂H₁₉Cl₂F₃N₆O₄S] [M+H]⁺: 591.1; found: 590.8. ¹H NMR (400 MHz, Methanol-d₄) δ 8.50 (s, 1H), 7.80 (d, J=8.8 Hz, 1H), 7.69 (dd, J=8.4, 1.6 Hz, 1H), 7.62 (br s, 1H), 7.36 (s, 1H), 6.46 (s, 1H), 4.74 (m, 1H), 4.62-4.37 (m, 1H), 4.14-3.93 (m, 2H), 3.60-3.38 (m, 3H), 2.22 (s, 3H).

### Example 10

### 1-{5-{3-[4-(5-Chlorothiazol-2-yl)-1H-1,2,3-triazol-1-yl]-3-deoxy-β-D-galactopyranosyl}-1H-1,2-pyrazol-1-yl}-5-chloro-2-(trifluoromethyl)benzene

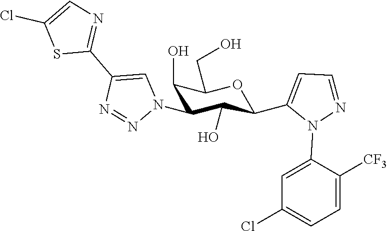

A solution of 3,7-anhydro-6,8-O-benzylidene-5-[4-(5-chlorothiazol-2-yl)-1H-1,2,3-triazol-1-yl]-5-deoxy-4-O-(methoxymethyl)-D-glycero-L-manno-2-octulose (90 mg, 0.18 mmol) in N,N-dimethylformamide dimethyl acetal (2.0 mL) was stirred 16 h at 80° C. The mixture was concentrated to dryness and the residue was dissolved in EtOH (5.0 mL). [5-Chloro-2-(trifluoromethyl)phenyl]hydrazine (74.8 mg, 0.36 mmol) and concentrated HCl (0.1 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (33.5 mg, 32%). ESI-MS m/z calcd for [C₂₁H₁₇Cl₂F₃N₆O₄S] [M+H]⁺: 577.0; found: 576.8. ¹H NMR (400 MHz, Methanol-d₄) δ 8.60 (s, 1H), 7.92 (d, J=8.8 Hz, 1H), 7.80 (d, J=8.4 Hz, 1H), 7.79-7.64 (m, 2H), 7.46 (s, 1H), 6.77 (d, J=1.6 Hz, 1H), 4.93-4.88 (m, 1H), 4.66 (br s, 1H), 4.22-4.17 (m, 1H), 4.11 (d, J=2.8 Hz, 1H), 3.69-3.62 (m, 3H).

### Example 11

### 1-{5-{3-[4-(4-Chlorothiazol-2-yl)-1H-1,2,3-triazol-1-yl]-3-deoxy-β-D-galactopyranosyl}-1H-1,2-pyrazol-1-yl}-5-chloro-2-(trifluoromethyl)benzene

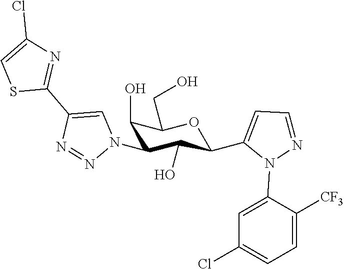

A solution of 3,7-anhydro-6,8-O-benzylidene-5-[4-(5-chlorothiazol-2-yl)-1H-1,2,3-triazol-1-yl]-5-deoxy-4-O-(methoxymethyl)-D-glycero-L-manno-2-octulose (100 mg, 0.20 mmol) in N,N-dimethylformamide dimethyl acetal (3.0 mL) was stirred 2 h at 80° C. The mixture was concentrated to dryness and the residue was dissolved together with [5-chloro-2-(trifluoromethyl)phenyl]hydrazine (82.4 mg, 0.39 mmol) in EtOH (5.0 mL). Concentrated HCl (0.5 mL) was added and the mixture was stirred overnight at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (22.8 mg, 20%). ESI-MS m/z calcd for [C₂₁H₁₇Cl₂F₃N₆O₄S] [M+H]⁺: 577.0; found: 577.2. ¹H NMR (400 MHz, Methanol-d₄) δ 8.61 (s, 1H), 7.92 (d, J=8.4 Hz, 1H), 7.80 (d, J=8.4 Hz, 1H), 7.79-7.73 (m, 2H), 7.46 (s, 1H), 6.78 (d, J=2.0 Hz, 1H), 4.87-4.85 (m, 1H), 4.65-4.58 (m, 1H), 4.20 (d, J=8.8 Hz, 1H), 4.12 (d, J=2.8 Hz, 1H), 3.69-3.58 (m, 3H).

### Example 12

### 1-{5-{3-[4-(5-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene

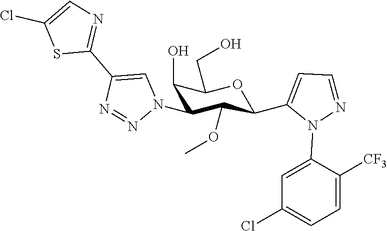

A solution of 3,7-anhydro-6,8-O-benzylidene-5-[4-(5-chlorothiazol-2-yl)-1H-1,2,3-triazol-1-yl]-5-deoxy-4-O-methyl-D-glycero-L-manno-2-octulose (120 mg, 0.25 mmol) in N,N-dimethylformamide dimethyl acetal (2.0 mL) was stirred 3 h at 100° C. The mixture was concentrated to dryness and the residue was dissolved in EtOH (5.0 mL). [5-Chloro-2-(trifluoromethyl)phenyl]hydrazine (92.7 mg, 0.44 mmol) and concentrated HCl (0.2 mL) were added and the mixture was stirred 3 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (45.7 mg, 26%). ESI-MS m/z calcd for [C₂₂H₁₉Cl₂F₃N₆O₄S] [M+H]⁺: 591.1; found: 590.8. ¹H NMR (400 MHz, Methanol-d₄) δ 8.67 (s, 1H), 7.84 (d, J=8.8 Hz, 1H), 7.72 (d, J=8.8 Hz, 1H), 7.71-7.43 (m, 2H), 7.38 (s, 1H), 6.79 (d, J=1.6 Hz, 1H), 4.85 (dd, J=10.4, 2.8 Hz, 1H), 4.45 (br s, 1H), 4.09 (d, J=9.2 Hz, 1H), 3.98 (s, 1H), 3.60-3.53 (m, 3H), 2.86 (s, 3H).

### Example 13

### 1-{5-{3-[4-(4-Chlorothiazol-2-yl)-1H-1,2,3-triazol-1-yl]-3-deoxy-2-O-methyl-β-D-galactopyranosyl}-3-methyl-1H-1,2-pyrazol-1-yl}-5-chloro-2-(trifluoromethyl)benzene

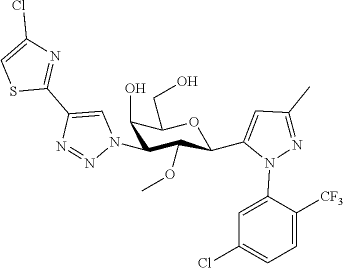

A solution of 3,7-anhydro-6,8-O-benzylidene-5-[4-(4-chlorothiazol-2-yl)-1H-1,2,3-triazol-1-yl]-5-deoxy-4-O-methyl-D-glycero-L-manno-2-octulose (140 mg, 0.29 mmol) in N,N-dimethylacetamide dimethyl acetal (2.0 mL) was stirred 3 h at 100° C. The mixture was concentrated to dryness and the residue was dissolved in EtOH (5.0 mL). [5-Chloro-2-(trifluoromethyl)phenyl]hydrazine (92.7 mg, 0.44 mmol) and concentrated HCl (0.2 mL) were added and the mixture was stirred 3 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (22.4 mg, 13%). ESI-MS m/z calcd for [C₂₃H₂₁Cl₂F₃N₆O₄S] [M+H]⁺: 605.1; found: 604.8. ¹H NMR (400 MHz, Methanol-d₄) δ 8.74 (s, 1H), 7.92 (dd, J=8.8 Hz, 1H), 7.80 (dd, J=8.4, 1.2 Hz, 1H), 7.66 (s, 1H), 7.46 (s, 1H), 6.66 (s, 1H), 4.92 (dd, J=10.4, 3.2 Hz, 1H), 4.40 (br s, 1H), 4.15 (d, J=9.6 Hz, 1H), 4.12 (d, J=2.4 Hz, 1H), 3.71-3.61 (m, 3H), 2.99 (s, 3H), 2.35 (s, 3H).

### Example 14

### 5-Chloro-1-{5-{3-deoxy-2-O-methyl-3-[4-(2-thiazolyl)-1H-1,2,3-triazol-1-yl]-β-D-galactopyranosyl}-3-methyl-1H-1,2-pyrazol-1-yl}-2-(trifluoromethyl)benzene

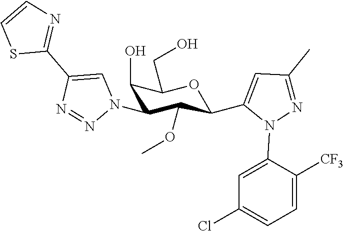

A solution of 3,7-anhydro-6,8-O-benzylidene-5-deoxy-4-O-methyl-5-[4-(2-thiazol-2-yl)-1H-1,2,3-triazol-1-yl]-D-glycero-L-manno-2-octulose (88 mg, 0.20 mmol) in N,N-dimethylacetamide dimethyl acetal (2.0 mL) was stirred overnight at 75° C. The mixture was concentrated to dryness and the residue was dissolved in EtOH (5.0 mL). [5-Chloro-2-(trifluoromethyl)phenyl]hydrazine (66 mg, 0.31 mmol) and concentrated HCl (0.2 mL) were added and the mixture was stirred 3 h at 60° C. The mixture was concentrated and purified by prep HPLC (MeCN/H₂O (10 mmol/L NH₄HCO₃), X-Select 10 μm 19*250 mm, 20 mL/min, UV 254) to afford the title compound (12.4 mg, 11%). ESI-MS m/z calcd for [C₂₃H₂₂ClF₃N₆O₄S] [M+H]⁺: 571.1; found: 570.8. ¹H NMR (400 MHz, Methanol-d₄) δ 8.72 (s, 1H), 7.93-7.63 (m, 5H), 6.66 (s, 1H), 4.92 (dd, J=10.4, 2.8 Hz, 1H), 4.41 (br s, 1H), 4.12 (d, J=9.2 Hz, 1H), 4.08 (d, J=1.6 Hz, 1H), 3.65-3.60 (m, 3H), 2.96 (s, 3H), 2.33 (s, 3H).

### Example 15

### 1-{5-{3-[4-(4-Chlorothiazol-2-yl)-1H-1,2,3-triazol-1-yl]-3-deoxy-2-O-methyl-β-D-galactopyranosyl}-3-methyl-1H-1,2-pyrazol-1-yl}-2-methylbenzothiazole

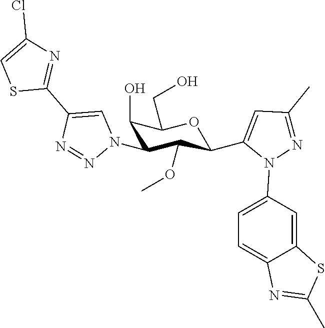

A solution of 3,7-anhydro-6,8-O-benzylidene-5-[4-(4-chlorothiazol-2-yl)-1H-1,2,3-triazol-1-yl]-5-deoxy-4-O-methyl-D-glycero-L-manno-2-octulose (100 mg, 0.21 mmol) in N,N-dimethylacetamide dimethyl acetal (5 mL) was stirred 3 h at 90° C. The mixture was concentrated to dryness and the residue was dissolved in EtOH (8.0 mL). (5-Methyl-1,3-benzothiazol-6-yl)hydrazine (75.2 mg, 0.42 mmol) and concentrated HCl (0.2 mL) were added and the mixture was stirred 2 h at 70° C. The mixture was concentrated and purified by prep HPLC (MeCN/H₂O (10 mmol/L NH₄HCO₃), X-Select 10 μm 19*250 mm, 20 m/min, UV 254) to afford the title compound (11 mg, 9%). ESI-MS m/z calcd for [C₂₄H₂₄ClN₇O₄S₂] [M+H]⁺: 574.1; found: 574.2. ¹H NMR (400 MHz, Methanol-d₄) δ 8.72 (s, 1H), 8.26 (d, J=2.0 Hz, 1H), 8.03 (d, J=8.8 Hz, 1H), 7.72 (dd, J=8.8, 2.0 Hz, 1H), 7.46 (s, 1H), 6.93 (s, 1H), 4.98-4.94 (m, 1H), 4.47-4.42 (m, 2H), 4.08 (d, J=2.4 Hz, 1H), 3.82-3.68 (m, 3H), 2.93 (s, 3H), 2.88 (s, 3H), 2.36 (s, 3H).

### Example 16

### 1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-2-methylbenzothiazole

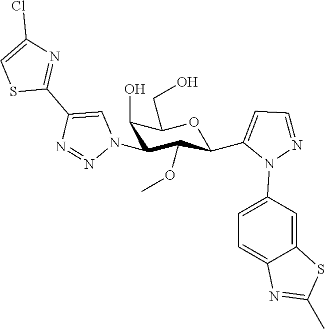

A solution of 3,7-anhydro-6,8-O-benzylidene-5-[4-(4-chlorothiazol-2-yl)-1H-1,2,3-triazol-1-yl]-5-deoxy-4-O-methyl-D-glycero-L-manno-2-octulose (100 mg, 0.21 mmol) in N,N-dimethylformamide dimethyl acetal (5 mL) was stirred 3 h at 90° C. The mixture was concentrated to dryness and the residue was dissolved in EtOH (8.0 mL). (5-Methyl-1,3-benzothiazol-6-yl)hydrazine (75.2 mg, 0.42 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (39 mg, 34%). ESI-MS m/z calcd for [C₂₃H₂₂ClN₇O₄S₂] [M+H]⁺: 560.1; found: 560.1. ¹H NMR (400 MHz, Methanol-d₄) δ 8.73 (s, 1H), 8.29 (d, J=2.0 Hz, 1H), 8.05 (d, J=8.8 Hz, 1H), 7.77 (d, J=2.0 Hz, 1H), 7.75 (dd, J=8.4, 2.0 Hz, 1H), 7.46 (s, 1H), 6.91 (d, J=2.0 Hz, 1H), 4.99-4.96 (m, 1H), 4.50-4.47 (m, 2H), 4.09 (d, J=2.8 Hz, 1H), 3.83-3.68 (m, 3H), 2.91 (s, 3H), 2.88 (s, 3H).

### Example 17

### 1-{5-{3-[4-(4-Chlorothiazol-2-yl)-1H-1,2,3-triazol-1-yl]-3-deoxy-2-O-methyl-β-D-galactopyranosyl}-3-ethoxycarbonyl-1H-1,2-pyrazol-1-yl}-5-chloro-2-(trifluoromethyl)benzene

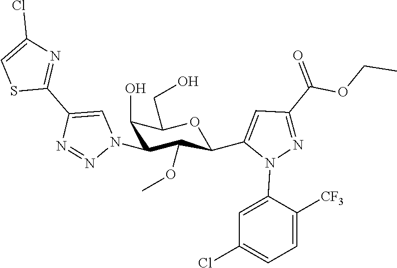

To a solution of 1-[5-(3-azido-3-deoxy-2-O-methyl-3-D-galactopyranosyl)-3-ethoxycarbonyl-1H-1,2-pyrazol-1-yl]-5-chloro-2-(trifluoromethyl)benzene (62 mg, 0.12 mmol) in DMF (3 mL) 2-(4-chlorothiazol-2-yl)ethynyltrimethylsilane (51.5 mg, 0.24 mmol), copper(II) sulfate pentahydrate (29.8 mg, 0.12 mmol) and (+)-sodium L-ascorbate (23.6 mg, 0.12 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (44 mg, 56%). ESI-MS m/z calcd for [C₂₅H₂₃Cl₂F₃N₆O₆S] [M+H]⁺: 663.1; found: 663.1. ¹H NMR (400 MHz, Methanol-d₄) δ 8.78 (s, 1H), 7.96 (d, J=8.4 Hz, 1H), 7.87 (d, J=8.4 Hz, 1H), 7.86-7.52 (m, 1H), 7.47 (s, 1H), 7.34 (s, 1H), 4.97 (dd, J=10.4, 2.8 Hz, 1H), 4.65-4.55 (m, 1H), 4.40 (q, J=7.2 Hz, 2H), 4.24-4.20 (m, 1H), 4.07 (br s, 1H), 3.69-3.60 (m, 3H), 3.01-2.91 (m, 3H), 1.40 (t, J=7.2 Hz, 3H).

### Example 18

### 1-{3-Carboxy-5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene

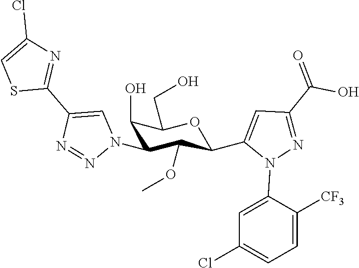

To a solution of 1-{5-{3-[4-(4-chlorothiazol-2-yl)-1H-1,2,3-triazol-1-yl]-3-deoxy-2-O-methyl-β-D-galactopyranosyl}-3-ethoxycarbonyl-1H-1,2-pyrazol-1-yl}-5-chloro-2-(trifluoromethyl)benzene (35 mg, 0.053 mmol) in MeOH/THF/H₂O (6.00 mL, 1:1:1) NaOH (20.0 mg, 0.500 mmol) was added and the mixture was stirred 2 h at rt. Acetic acid (0.1 mL) was added. The mixture was concentrated and purified by prep HPLC [MeCN/H₂O (0.1% TFA), X-Select 10 μm 19*250 mm, 20 m/min, UV 254] to afford the title compound (10.9 mg, 33%). ESI-MS m/z calcd for [C₂₃H₁₉Cl₂F₃N₆O₆S][M+H]⁺: 635.0; found: 635.0. ¹H NMR (400 MHz, Methanol-d₄) δ 8.79 (br s, 1H), 7.95 (d, J=8.8 Hz, 1H), 7.87-7.57 (m, 2H), 7.47 (s, 1H), 7.29 (s, 1H), 4.96 (dd, J=10.0, 2.0 Hz, 1H), 4.62-4.53 (m, 1H), 4.22-4.18 (m, 1H), 4.09-4.06 (m, 1H), 3.71-3.60 (m, 3H), 3.00-2.91 (m, 3H).

### Example 19

### 5-Bromo-1-{5-{3-[4-(4-chlorothiazol-2-yl)-1H-1,2,3-triazol-1-yl]-3-deoxy-β-D-galactopyranosyl}-1H-1,2-pyrazol-1-yl}-2-(trifluoromethyl)benzene

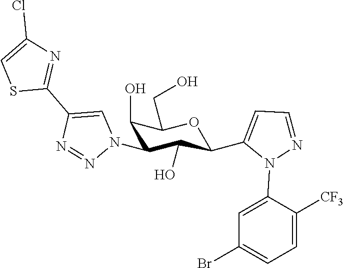

A solution of 3,7-anhydro-6,8-O-benzylidene-5-[4-(5-chlorothiazol-2-yl)-1H-1,2,3-triazol-1-yl]-5-deoxy-4-O-(methoxymethyl)-D-glycero-L-manno-2-octulose (100 mg, 0.20 mmol) in N,N-dimethylformamide dimethyl acetal (3.0 mL) was stirred 2 h at 80° C. The mixture was concentrated to dryness and the residue was dissolved together with [5-bromo-2-(trifluoromethyl)phenyl]hydrazine (99.8 mg, 0.39 mmol) in EtOH (5.0 mL). Concentrated HCl (0.5 mL) was added and the mixture was stirred overnight at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (8.2 mg, 7%). ESI-MS m/z calcd for [C₂₁H₁₇BrClF₃N₆O₄S] [M+H]⁺: 621.0; found: 621.0. ¹H NMR (400 MHz, Methanol-d₄) δ 8.61 (s, 1H), 7.96 (dd, J=8.4, 1.2 Hz, 1H), 7.88 (s, 1H), 7.84 (d, J=8.4 Hz, 1H), 7.73 (d, J=2.0 Hz, 1H), 7.46 (s, 1H), 6.77 (d, J=2.0 Hz, 1H), 4.87-4.86 (m, 1H), 4.69-4.63 (m, 1H), 4.20 (d, J=8.8 Hz, 1H), 4.12 (d, J=2.8 Hz, 1H), 3.69-3.59 (m, 3H).

### Example 20

### 5-Bromo-1-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-2-(trifluoromethyl)benzene

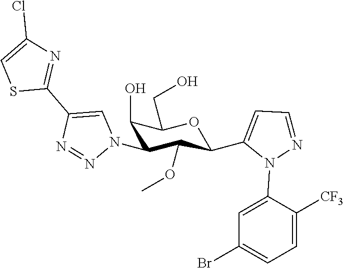

To a solution of 4,8-anhydro-7,9-O-benzylidene-6-[4-(4-chlorothiazol-2-yl)-1H-1,2,3-triazol-1-yl]-6-deoxy-5-O-methyl-1-N,N-dimethylamino-D-glycero-L-manno-non-1-en-3-ulose (70 mg, 0.13 mmol) in EtOH (5 mL) [5-bromo-2-(trifluoromethyl)phenyl]hydrazine (50.3 mg, 0.20 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (23.1 mg, 28%). ESI-MS m/z calcd for [C₂₂H₁₉BrClF₃N₆O₄S] [M+H]⁺: 635.0; found: 635.1. ¹H NMR (400 MHz, Methanol-d₄) δ 8.78 (s, 1H), 8.00 (d, J=8.4 Hz, 1H), 7.98-7.74 (m, 3H), 7.49 (s, 1H), 6.90 (d, J=2.0 Hz, 1H), 4.96 (dd, J=10.4, 2.8 Hz, 1H), 4.53 (br s, 1H), 4.21 (d, J=9.2 Hz, 1H), 4.11 (d, J=2.4 Hz, 1H), 3.72-3.63 (m, 3H), 2.97 (s, 3H).

### Example 21

### 1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-4,5-dichloro-2-(trifluoromethyl)benzene

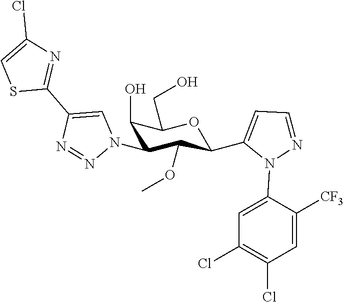

To a solution of 4,8-anhydro-7,9-O-benzylidene-6-[4-(4-chlorothiazol-2-yl)-1H-1,2,3-triazol-1-yl]-6-deoxy-5-O-methyl-1l-N,N-dimethylamino-D-glycero-L-manno-non-1-en-3-ulose (52 mg, 0.10 mmol) in EtOH (5 mL) [4,5-dichloro-2-(trifluoromethyl)phenyl]hydrazine (35.9 mg, 0.15 mmol) and concentrated HCl (0.2 mL) were added and the mixture was stirred 2 h at 80′° C. The mixture was concentrated and purified by prep HPLC (MeCN/H₂O (10 mmol/L NH₄HCO₃), X-Select 10 μm 19*250 mm, 20 mL/min, UV 254) to afford the title compound (19.3 mg, 32%). ESI-MS m/z calcd for [C₂₂H₁₈Cl₃F₃N₆O₄S] [M+H]⁺: 625.0; found: 625.0. ¹H NMR (400 MHz, Methanol-d₄) δ 8.76 (s, 1H), 8.14 (s, 1H), 7.94-7.73 (m, 2H), 7.47 (s, 1H), 6.87 (d, J=2.0 Hz, 1H), 4.96 (dd, J=10.4, 2.4 Hz, 1H), 4.46 (br s, 1H), 4.24 (d, J=8.8 Hz, 1H), 4.07 (d, J=2.0 Hz, 1H), 3.70-3.60 (m, 3H), 2.95 (s, 3H).

### Example 22

### 1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-4-fluoro-2-(trifluoromethyl)benzene

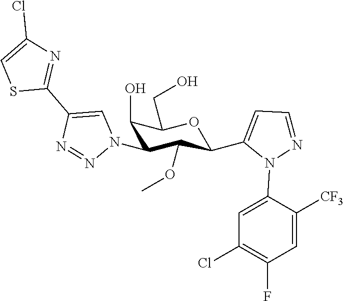

To a solution of 4,8-anhydro-7,9-O-benzylidene-6-[4-(4-chlorothiazol-2-yl)-1H-1,2,3-triazol-1-yl]-6-deoxy-5-O-methyl-1-N,N-dimethylamino-D-glycero-L-manno-non-1-en-3-ulose (82 mg, 0.15 mmol) in EtOH (5 mL) [5-chloro-4-fluoro-2-(trifluoromethyl)phenyl]hydrazine (52.8 mg, 0.15 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (27.4 mg, 29%). ESI-MS m/z calcd for [C₂₂H₁₈Cl₂F₄N₆O₄S] [M+H]⁺: 609.0; found: 609.1. ¹H NMR (400 MHz, Methanol-d₄) δ 8.76 (s, 1H), 7.93-7.74 (m, 3H), 7.47 (s, 1H), 6.86 (d, J=2.0 Hz, 1H), 4.95 (dd, J=10.4, 2.8 Hz, 1H), 4.51 (br s, 1H), 4.24-4.20 (m, 1H), 4.07 (s, 1H), 3.72-3.61 (m, 3H), 2.95 (s, 3H).

### Example 23

### 5-Bromo-1-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-4-fluoro-2-(trifluoromethyl)benzene

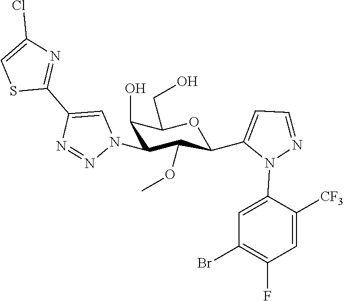

To a solution of 4,8-anhydro-7,9-O-benzylidene-6-[4-(4-chlorothiazol-2-yl)-1H-1,2,3-triazol-1-yl]-6-deoxy-5-O-methyl-1-N,N-dimethylamino-D-glycero-L-manno-non-1-en-3-ulose (72 mg, 0.14 mmol) in EtOH (5 mL) [5-bromo-4-fluoro-2-(trifluoromethyl)phenyl]hydrazine (55.4 mg, 0.20 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (17.0 mg, 19%). ESI-MS m/z calcd for [C₂₂H₁₈BrClF₄N₆O₄S] [M+H]⁺: 653.0; found: 653.0. ¹H NMR (400 MHz, DMSO-d₆) δ 9.00 (s, 1H), 8.13 (d, J=8.8 Hz, 1H), 8.02-7.96 (m, 1H), 7.80 (s, 1H), 7.77 (d, J=2.0 Hz, 1H), 6.83 (br s, 1H), 5.38 (d, J=6.4 Hz, 1H), 5.06 (dd, J=10.0, 2.4 Hz, 1H), 4.71 (d, J=10.4 Hz, 1H), 4.64-4.60 (m, 1H), 4.23 (d, J=9.2 Hz, 1H), 3.88 (dd, J=6.4, 2.8 Hz, 1H), 3.63 (t, J=5.6 Hz, 1H), 3.44-3.37 (m, 2H), 2.88 (s, 3H).

### Example 24

### 5-Bromo-1-{5-{3-[4-(4-chlorothiazol-2-yl)-1H-1,2,3-triazol-1-yl]-3-deoxy-β-D-galactopyranosyl}-1H-1,2-pyrazol-1-yl}-4-fluoro-2-(trifluoromethyl)benzene

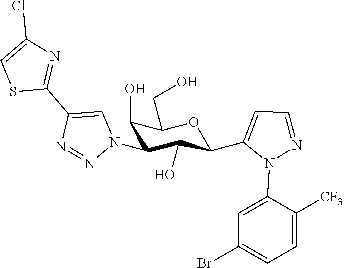

To a solution of 4,8-anhydro-7,9-O-benzylidene-6-[4-(4-chlorothiazol-2-yl)-1H-1,2,3-triazol-1-yl]-6-deoxy-5-O-(methoxymethyl)-1-N,N-dimethylamino-D-glycero-L-manno-non-1-en-3-ulose (56 mg, 0.10 mmol) in EtOH (5 mL) [5-bromo-4-fluoro-2-(trifluoromethyl)phenyl]hydrazine (27.2 mg, 0.10 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (8.75 mg, 14%). ESI-MS m/z calcd for [C₂₁H₁₆BrClF₄N₆O₄S] [M+H]⁺: 639.0; found: 639.0. ¹H NMR (400 MHz, Methanol-d₄) δ 8.60 (s, 1H), 8.01 (br s, 1H), 7.85 (d, J=8.4 Hz, 1H), 7.73 (d, J=2.0 Hz, 1H), 7.46 (s, 1H), 6.76 (d, J=1.6 Hz, 1H), 4.90-4.87 (m, 1H), 4.61 (br s, 1H), 4.22 (d, J=7.6 Hz, 1H), 4.11 (d, J=2.8 Hz, 1H), 3.65-3.60 (m, 3H).

### Example 25

### 1-{5-{3-[4-(4-Chlorothiazol-2-yl)-1H-1,2,3-triazol-1-yl]-3-deoxy-2-O-methyl-β-D-galactopyranosyl}-3-chloro-1H-1,2-pyrazol-1-yl}-5-chloro-2-(trifluoromethyl)benzene

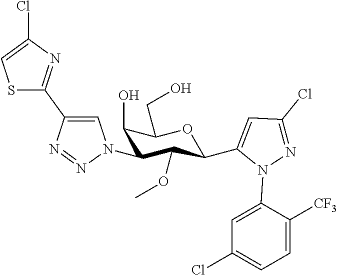

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-chloro-1H-1,2-pyrazol-1-yl}-5-chloro-2-(trifluoromethyl)benzene (69 mg, 0.097 mmol) in DCM (4 mL) TFA (0.2 mL) was added and the mixture was stirred overnight at rt. Et₃N (1 mL) was added at 0° C. The mixture was concentrated and purified by prep HPLC (MeCN/H₂O (10 mmol/L NH₄HCO₃), X-Select 10 μM 19*250 mm, 20 m/min, UV 254) to afford the title compound (43.1 mg, 71%). ESI-MS m/z calcd for [C₂₂H₁₈Cl₃F₃N₆O₄S] [M+H]⁺: 625.0; found: 625.1. ¹H NMR (400 MHz, Methanol-d₄) δ 8.76 (s, 1H), 7.93 (d, J=8.8 Hz, 1H), 7.92-7.62 (m, 2H), 7.47 (s, 1H), 6.86 (s, 1H), 4.95 (dd, J=10.4, 2.8 Hz, 1H), 4.52-4.37 (m, 1H), 4.17 (d, J=9.2 Hz, 1H), 4.07 (d, J=2.0 Hz, 1H), 3.69-3.62 (m, 3H), 2.97 (s, 3H).

### Example 26

### 1-{3-Bromo-5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene

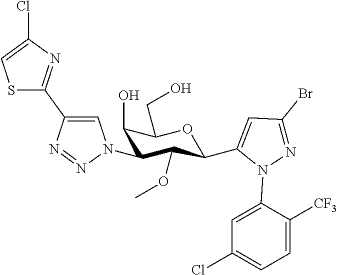

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-bromo-1H-1,2-pyrazol-1-yl}-5-chloro-2-(trifluoromethyl)benzene (71 mg, 0.094 mmol) in DCM (6 mL) TFA (0.2 mL) was added and the mixture was stirred overnight at rt. Et₃N (1 mL) was added at 0° C. The mixture was concentrated and purified by prep HPLC (MeCN/H₂O (10 mmol/L NH₄HCO₃), X-Select 10 μM 19*250 mm, 20 m/min, UV 254) to afford the title compound (25.6 mg, 41%). ESI-MS m/z calcd for [C₂₂H₁₈BrCl₂F₃N₆O₄S] [M+H]⁺: 669.0; found: 668.9. ¹H NMR (400 MHz, Methanol-d₄) δ 8.75 (s, 1H), 7.93 (d, J=8.4 Hz, 1H), 7.92-7.61 (m, 2H), 7.47 (s, 1H), 6.93 (s, 1H), 4.94 (dd, J=10.4, 3.2 Hz, 1H), 4.55-4.35 (m, 1H), 4.18 (d, J=8.8 Hz, 1H), 4.07 (d, J=2.0 Hz, 1H), 3.69-3.60 (m, 3H), 2.97 (s, 3H).

### Example 27

### 1-{5-{3-[4-(4-Chlorothiazol-2-yl)-1H-1,2,3-triazol-1-yl]-3-deoxy-2-O-methyl-β-D-galactopyranosyl}-3-(1-methylethenyl)-1H-1,2-pyrazol-1-yl}-5-chloro-2-(trifluoromethyl)benzene

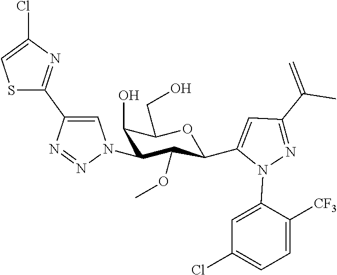

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-(1-methylethenyl)-1H-1,2-pyrazol-1-yl}-5-chloro-2-(trifluoromethyl)benzene (19 mg, 0.026 mmol) in DCM (5 mL) TFA (0.2 mL) was added and the mixture was stirred overnight at rt. Et₃N (0.5 mL) was added at 0° C. The mixture was concentrated and purified by prep HPLC (MeCN/H₂O (10 mmol/L NH₄HCO₃), X-Select 10 μM 19*250 mm, 20 mL/min, UV 254) to afford the title compound (9.3 mg, 56%). ESI-MS m/z calcd for [C₂₅H₂₃Cl₂F₃N₆O₄S] [M+H]⁺: 631.1; found: 631.2. ¹H NMR (400 MHz, Methanol-d₄) δ 8.77 (s, 1H), 7.93 (d, J=8.8 Hz, 1H), 7.83-7.71 (m, 2H), 7.47 (s, 1H), 7.05 (s, 1H), 5.64 (s, 1H), 5.18 (s, 1H), 4.94 (dd, J=10.4, 2.8 Hz, 1H), 4.53-4.37 (m, 1H), 4.16 (d, J=9.2 Hz, 1H), 4.08 (d, J=2.4 Hz, 1H), 3.72-3.63 (m, 3H), 2.97 (s, 3H), 2.12 (s, 3H).

### Example 28

### 1-{5-{3-[4-(4-Chlorothiazol-2-yl)-1H-1,2,3-triazol-1-yl]-3-deoxy-2-O-methyl-β-D-galactopyranosyl}-3-isopropyl-1H-1,2-pyrazol-1-yl}-5-chloro-2-(trifluoromethyl)benzene

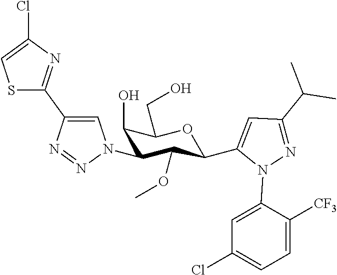

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-isopropyl-1H-1,2-pyrazol-1-yl}-5-chloro-2-(trifluoromethyl)benzene (36 mg, 0.050 mmol) in DCM (3 mL) TFA (0.15 mL) was added and the mixture was stirred overnight at rt. Et₃N (0.5 mL) was added at 0° C. The mixture was concentrated and purified by prep HPLC (MeCN/H₂O (10 mmol/L NH₄HCO₃), X-Select 10 μM 19*250 mm, 20 mL/min, UV 254) to afford the title compound (8.07 mg, 26%). ESI-MS m/z calcd for [C₂₅H₂₅Cl₂F₃N₆O₄S] [M+H]⁺: 633.1; found: 633.3. ¹H NMR (400 MHz, Methanol-d₄) δ 8.75 (s, 1H), 7.92 (d, J=8.8 Hz, 1H), 7.91-7.65 (m, 2H), 7.47 (s, 1H), 6.74 (s, 1H), 4.92 (dd, J=10.2, 2.8 Hz, 1H), 4.53-4.37 (m, 1H), 4.13 (d, J=9.6 Hz, 1H), 4.07 (d, J=2.8 Hz, 1H), 3.72-3.61 (m, 3H), 3.06-2.94 (m, 4H), 1.31 (dd, J=7.2, 1.2 Hz, 6H).

### Example 29

### 1-{5-{3-[4-(4-Chlorothiazol-2-yl)-1H-1,2,3-triazol-1-yl]-3-deoxy-2-O-methyl-β-D-galactopyranosyl}-3-ethenyl-1H-1,2-pyrazol-1-yl}-5-chloro-2-(trifluoromethyl)benzene

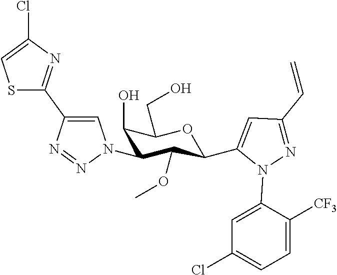

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-ethenyl-1H-1,2-pyrazol-1-yl}-5-chloro-2-(trifluoromethyl)benzene (33 mg, 0.047 mmol) in DCM (5 mL) TFA (0.2 mL) was added and the mixture was stirred overnight at rt. Et₃N (0.5 mL) was added at 0° C. The mixture was concentrated and purified by prep HPLC (MeCN/H₂O (10 mmol/L NH₄HCO₃), X-Select 10 μM 19*250 mm, 20 mL/min, UV 254) to afford the title compound (13 mg, 45%). ESI-MS m/z calcd for [C₂₄H₂₁Cl₂F₃N₆O₄S] [M+H]⁺: 617.1; found: 617.2. ¹H NMR (400 MHz, Methanol-d₄) δ 8.76 (s, 1H), 7.93 (d, J=8.8 Hz, 1H), 7.83-7.62 (m, 2H), 7.47 (s, 1H), 7.06 (s, 1H), 6.71 (dd, J=17.6, 11.2 Hz, 1H), 5.92 (dd, J=17.6, 1.2 Hz, 1H), 5.42 (dd, J=11.2, 1.2 Hz, 1H), 4.94 (dd, J=10.8, 3.2 Hz, 1H), 4.53-4.37 (m, 1H), 4.15 (d, J=9.2 Hz, 1H), 4.08 (d, J=2.4 Hz, 1H), 3.72-3.63 (m, 3H), 2.97 (s, 3H).

### Example 30

### 1-{5-{3-[4-(4-Chlorothiazol-2-yl)-1H-1,2,3-triazol-1-yl]-3-deoxy-2-O-methyl-β-D-galactopyranosyl}-3-ethyl-1H-1,2-pyrazol-1-yl}-5-chloro-2-(trifluoromethyl)benzene

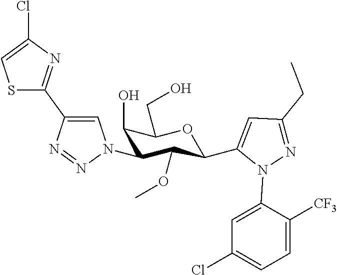

To a solution of 1-{5-{3-[4-(4-chlorothiazol-2-yl)-1H-1,2,3-triazol-1-yl]-3-deoxy-2-O-methyl-β-D-galactopyranosyl}-3-ethenyl-1H-1,2-pyrazol-1-yl}-5-chloro-2-(trifluoromethyl)benzene (10 mg, 0.016 mmol) in MeOH (10 mL) platinum(IV) oxide (4.05 mg, 0.018 mmol) was added and the mixture was stirred 5 h at rt under hydrogen atmosphere. The mixture was filtered through a celite pad and concentrated. The residue was purified by prep HPLC (MeCN/H₂O (10 mmol/L NH₄HCO₃), X-Select 10 μM 19*250 mm, 20 m/min, UV 254) to afford the title compound (2.6 mg, 26%). ESI-MS m/z calcd for [C₂₄H₂₃Cl₂F₃N₆O₄S] [M+H]⁺: 619.1; found: 619.2. ¹H NMR (400 MHz, Methanol-d₄) δ 8.75 (s, 1H), 7.92 (d, J=8.4 Hz, 1H), 7.81-7.65 (m, 2H), 7.47 (s, 1H), 6.72 (s, 1H), 4.92 (dd, J=10.4, 2.8 Hz, 1H), 4.51-4.35 (m, 1H), 4.13 (d, J=9.2 Hz, 1H), 4.07 (d, J=2.4 Hz, 1H), 3.70-3.60 (m, 3H), 2.97 (s, 3H), 2.71 (q, J=7.6 Hz, 2H), 1.29 (t, J=7.6 Hz, 3H).

### Example 31

### 1-{5-{3-[4-(4-Chlorothiazol-2-yl)-1H-1,2,3-triazol-1-yl]-3-deoxy-2-O-methyl-β-D-galactopyranosyl}-3-cyano-1H-1,2-pyrazol-1-yl}-5-chloro-2-(trifluoromethyl)benzene

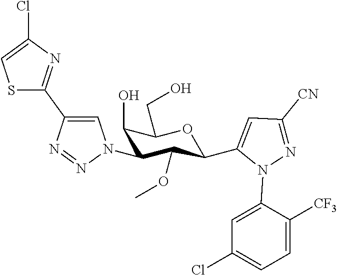

To a solution of 1-{5-{4,6-O-benzylidene-3-[4-(4-chlorothiazol-2-yl)-1H-1,2,3-triazol-1l-yl]-3-deoxy-2-O-methyl-β-D-galactopyranosyl}-3-cyano-11H-1,2-pyrazol-1l-yl}-5-chloro-2-(trifluoromethyl)benzene (33 mg, 0.047 mmol) in DCM (3 mL) TFA (0.2 mL) was added and the mixture was stirred overnight at rt. The mixture was concentrated and purified by prep HPLC (MeCN/H₂O (10 mmol/L NH₄HCO₃), X-Select 10 μM 19*250 mm, 20 mL/min, UV 254) to afford the title compound (9.4 mg, 33%). ESI-MS m/z calcd for [C₂₃H₁₈Cl₂F₃N₇O₄S] [M+H]⁺: 616.1; found: 616.1. ¹H NMR (400 MHz, Methanol-d₄) δ 8.78 (s, 1H), 7.97 (d, J=8.4 Hz, 1H), 7.96-7.64 (m, 2H), 7.47 (s, 1H), 7.38 (s, 1H), 4.98 (dd, J=10.4, 2.8 Hz, 1H), 4.54-4.49 (m, 1H), 4.29-4.24 (m, 1H), 4.07 (s, 1H), 3.69-3.59 (m, 3H), 2.94 (s, 3H).

### Example 32

### 3-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)pyridine

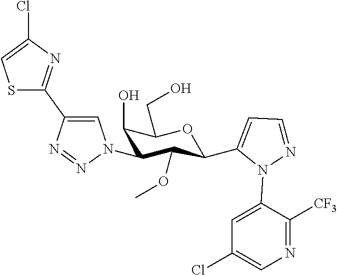

To a solution of 4,8-anhydro-7,9-O-benzylidene-6-[4-(4-chlorothiazol-2-yl)-1H-1,2,3-triazol-1-yl]-6-deoxy-5-O-methyl-1-N,N-dimethylamino-D-glycero-L-manno-non-1-en-3-ulose (82 mg, 0.15 mmol) in EtOH (5 mL) [5-chloro-2-(trifluoromethyl)-3-pyridyl]hydrazine (48.9 mg, 0.23 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (20.4 mg, 28%). ESI-MS m/z calcd for [C₂₁H₁₈Cl₂F₃N₇O₄S] [M+H]⁺: 592.1; found: 592.0. ¹H NMR (400 MHz, Methanol-d₄) δ 8.91 (d, J=2.0 Hz, 1H), 8.75 (s, 1H), 8.28 (br s, 1H), 7.79 (d, J=1.6 Hz, 1H), 7.47 (s, 1H), 6.87 (d, J=2.0 Hz, 1H), 4.97 (dd, J=10.0, 2.8 Hz, 1H), 4.43-4.33 (m, 2H), 4.06 (d, J=2.8 Hz, 1H), 3.69-3.57 (m, 3H), 2.95 (s, 3H).

### Example 33

### 5-Bromo-3-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-2-(trifluoromethyl)pyridine

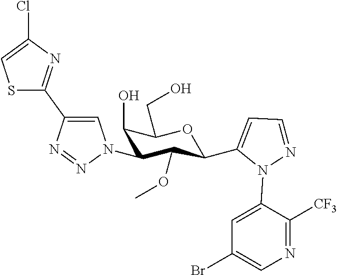

A solution of 3,7-anhydro-6,8-O-benzylidene-5-[4-(4-chlorothiazol-2-yl)-1H-1,2,3-triazol-1-yl]-5-deoxy-4-O-methyl-D-glycero-L-manno-2-octulose (90 mg, 0.19 mmol) in N,N-dimethylformamide dimethyl acetal (2.0 mL) was stirred 3 h at 100° C. The mixture was concentrated to dryness and the residue was dissolved in EtOH (5.0 mL). [5-Bromo-2-(trifluoromethyl)-3-pyridyl]hydrazine (73 mg, 0.14 mmol) and concentrated HCl (0.5 mL) were added and the mixture was stirred 3 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (13 mg, 15%). ESI-MS m/z calcd for [C₂₁H₁₈BrClF₃N₇O₄S] [M+H]⁺: 636.0; found: 636.0. ¹H NMR (400 MHz, Methanol-d₄) δ 9.01 (s, 1H), 8.75 (s, 1H), 8.42 (s, 1H), 7.79 (s, 1H), 7.47 (s, 1H), 6.87 (s, 1H) 4.98-4.94 (m, 1H), 4.42-4.38 (m, 1H), 4.34 (d, J=9.2 Hz, 1H), 4.06 (d, J=2.0 Hz, 1H), 3.68-3.55 (m, 3H), 2.94 (s, 3H).

### Example 34

### 1-{5-{3-[4-(2-Aminothiazol-4-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene

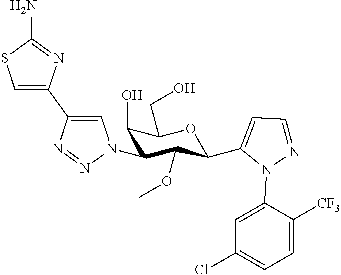

To a solution of 1-[5-(3-azido-3-deoxy-2-O-methyl-β-D-galactopyranosyl)-1H-1,2-pyrazol-1-yl]-5-chloro-2-(trifluoromethyl)benzene (22 mg, 0.049 mmol) in DMF (2 mL) 4-(2-trimethylsilylethynyl)thiazol-2-amine (14.5 mg, 0.074 mmol), copper(II) sulfate pentahydrate (12.3 mg, 0.049 mmol) and (+)-sodium L-ascorbate (9.7 mg, 0.049 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (7.3 mg, 26%). ESI-MS m/z calcd for [C₂₂H₂₁ClF₃N₇O₄S] [M+H]⁺: 572.1; found: 572.1 ¹H NMR (400 MHz, Methanol-d₄) δ 8.40 (br s, 1H), 7.95-7.75 (m, 4H), 6.97 (br s, 1H), 6.85 (d, J=2.0 Hz, 1H), 4.96-4.92 (m, 1H), 4.60-4.31 (m, 1H), 4.18 (d, J=9.2 Hz, 1H), 4.07 (s, 1H), 3.68-3.61 (m, 3H), 2.92 (s, 3H).

### Example 35

### 5-Chloro-1-{5-{3-deoxy-3-[4-(2-hydroxythiazol-4-yl)-1H-1,2,3-triazol-1-yl]-2-O-methyl-β-D-galactopyranosyl}-1H-1,2-pyrazol-1-yl}-2-(trifluoromethyl)benzene

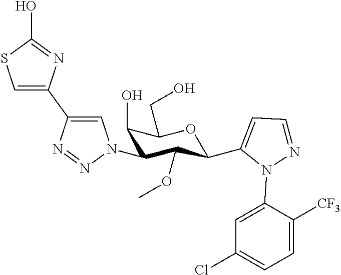

To a solution of 1-[5-(3-azido-3-deoxy-2-O-methyl-3-D-galactopyranosyl)-1H-1,2-pyrazol-1-yl]-5-chloro-2-(trifluoromethyl)benzene (22 mg, 0.049 mmol) in DMF (2 mL) 4-(2-trimethylsilylethynyl)thiazol-2-ol (14.5 mg, 0.074 mmol), copper(II) sulfate pentahydrate (12.3 mg, 0.049 mmol) and (+)-sodium L-ascorbate (9.7 mg, 0.049 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (9.7 mg, 35%). ESI-MS m/z calcd for [C₂₂H₂₀ClF₃N₆O₅S] [M+H]⁺: 573.1; found: 573.1 ¹H NMR (400 MHz, Methanol-d₄) δ 8.48 (s, 1H), 7.93 (d, J=8.4 Hz, 1H), 7.83-7.50 (m, 3H), 6.83 (d, J=1.6 Hz, 1H), 6.70 (s, 1H), 4.92-4.89 (m, 1H), 4.53-4.37 (m, 1H), 4.19 (d, J=9.2 Hz, 1H), 4.06 (d, J=2.4 Hz, 1H), 3.69-3.60 (m, 3H), 2.91 (s, 3H).

### Example 36

### 4,5-Dichloro-1-{5-{3-deoxy-3-[4-(2-hydroxythiazol-4-yl)-1H-1,2,3-triazol-1-yl]-2-O-methyl-β-D-galactopyranosyl}-1H-1,2-pyrazol-1-yl}-2-(trifluoromethyl)benzene

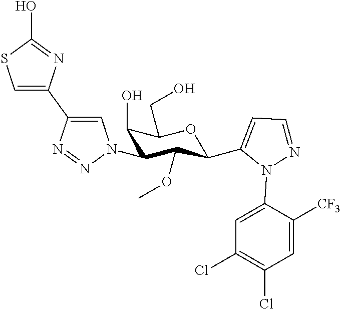

To a solution of 1-[5-(3-azido-3-deoxy-2-O-methyl-β-D-galactopyranosyl)-1H-1,2-pyrazol-1-yl]-4,5-dichloro-2-(trifluoromethyl)benzene (20 mg, 0.042 mmol) in DMF (1.5 mL) 4-(2-trimethylsilylethynyl)thiazol-2-ol (9.0 mg, 0.046 mmol), copper(II) sulfate pentahydrate (10.4 mg, 0.042 mmol) and (+)-sodium L-ascorbate (8.2 mg, 0.042 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (7.35 mg, 29%). ESI-MS m/z calcd for [C₂₂H₁₉Cl₂F₃N₆O₅S] [M+H]⁺: 607.1; found: 607.1 ¹H NMR (400 MHz, Methanol-d₄) δ 8.48 (s, 1H), 8.14 (s, 1H), 7.91-7.76 (m, 2H), 6.82 (d, J=2.0 Hz, 1H), 6.70 (s, 1H), 4.91 (dd, J=10.4, 2.4 Hz, 1H), 4.43-4.23 (m, 2H), 4.05 (d, J=2.4 Hz, 1H), 3.67-3.61 (m, 3H), 2.91 (s, 3H).

### Example 37

### 5-Bromo-1-{5-{3-deoxy-3-[4-(2-hydroxythiazol-4-yl)-1H-1,2,3-triazol-1-yl]-2-O-methyl-β-D-galactopyranosyl}-1H-1,2-pyrazol-1-yl}-4-fluoro-2-(trifluoromethyl)benzene

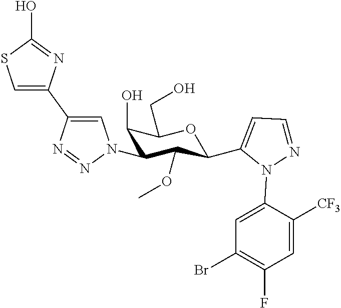

To a solution of 1-[5-(3-azido-3-deoxy-2-O-methyl-β-D-galactopyranosyl)-1H-1,2-pyrazol-1-yl]-5-bromo-4-fluoro-2-(trifluoromethyl)benzene (21 mg, 0.041 mmol) in DMF (1.5 mL) 4-(2-trimethylsilylethynyl)thiazol-2-ol (9 mg, 0.046 mmol), copper(II) sulfate pentahydrate (10.4 mg, 0.042 mmol) and (+)-sodium L-ascorbate (8.2 mg, 0.042 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (2.65 mg, 10%). ESI-MS m/z calcd for [C₂₂H₁₉BrF₄N₆O₅S] [M+H]⁺: 635.0; found: 635.0 ¹H NMR (400 MHz, Methanol-d₄) δ 8.48 (s, 1H), 8.05-7.71 (m, 3H), 6.81 (d, J=2.0 Hz, 1H), 6.70 (s, 1H), 4.92-4.90 (m, 1H), 4.52-4.21 (m, 2H), 4.05 (d, J=2.4 Hz, 1H), 3.67-3.61 (m, 3H), 2.91 (s, 3H).

### Example 38

### 5-Chloro-1-{5-{3-deoxy-2-O-methyl-3-[4-(4-thiazolyl)-1H-1,2,3-triazol-1-yl]-β-D-galactopyranosyl}-3-methyl-1H-1,2-pyrazol-1-yl}-2-(trifluoromethyl)benzene

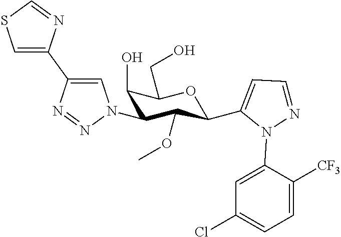

To a solution of 1-[5-(3-azido-3-deoxy-2-O-methyl-3-D-galactopyranosyl)-1H-1,2-pyrazol-1-yl]-5-chloro-2-(trifluoromethyl)benzene (30 mg, 0.067 mmol) in DMF (2 mL) trimethyl(2-thiazol-4-ylethynyl)silane (36.4 mg, 0.020 mmol), copper(II) sulfate pentahydrate (16.7 mg, 0.067 mmol) and (+)-sodium L-ascorbate (13.3 mg, 0.067 mmol) were added and the mixture was stirred overnight at rt. The mixture was filtered, and the filtrate was purified by prep HPLC (MeCN/H₂O (10 mmol/L NH₄HCO₃), X-Select 10 μm 19*250 mm, 20 mL/min, UV 254) to afford the title compound (14.9 mg, 40%). ESI-MS m/z calcd for [C₂₂H₂₀ClF₃N₆O₄S] [M+H]⁺: 557.1; found: 557.1 ¹H NMR (400 MHz, Methanol-d₄) δ 8.87-8.56 (m, 1H), 8.23-7.31 (m, 6H), 6.87 (d, J=1.6 Hz, 1H), 4.95-4.93 (m, 1H), 4.62-4.32 (m, 1H), 4.20-4.17 (m, 1H), 4.10 (br s, 1H), 3.70-3.61 (m, 3H), 2.94 (s, 3H).

### Example 39

### 1-{3-Aminocarbonyl-5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene

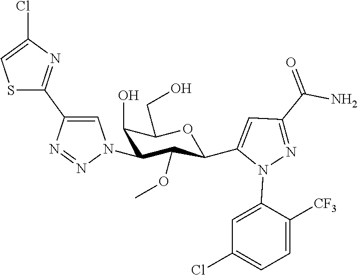

To a solution of 1-{-aminocarbonyl-5-{4,6-O-benzylidene-3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene (29 mg, 0.040 mmol) in DCM (3 mL) TFA (0.15 mL) was added and the mixture was stirred overnight at rt. The mixture was concentrated and purified by prep HPLC (MeCN/H₂O (10 mmol/L NH₄HCO₃), X-Select 10 μM 19*250 mm, 20 mL/min, UV 254) to afford the title compound (13.0 mg, 51%). ESI-MS m/z calcd for [C₂₃H₂₀Cl₂F₃N₇O₄S] [M+H]⁺: 634.1; found: 634.1. ¹H NMR (400 MHz, Methanol-d₄) δ 8.78 (s, 1H), 7.95 (d, J=8.4 Hz, 1H), 7.94-7.64 (m, 2H), 7.47 (s, 1H), 7.25 (s, 1H), 4.96 (dd, J=10.4, 2.8 Hz, 1H), 4.54-4.42 (m, 1H), 4.22 (d, J=9.2 Hz, 1H), 4.07 (d, J=2.8 Hz, 1H), 3.69-3.62 (m, 3H), 2.96 (s, 3H).

### Intermediate 1

### 2,4,6-Tri-O-acetyl-3-azido-3-deoxy-β-D-galactopyranosyl cyanide

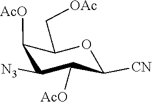

To a solution of 1,2,4,6-tetra-O-acetyl-3-azido-3-deoxy-β-D-galactopyranoside (10.0 g, 26.8 mmol) and trimethylsilyl cyanide (6.70 mL, 53.6 mmol) in nitromethane (100 mL) under argon at 0° C. boron trifluoride diethyl etherate (3.31 mL, 26.8 mmol) was added and the mixture was stirred 5 h at rt. The mixture was concentrated and purified by column chromatography (PE/EtOAc=5/1-2/1, Silica-CS 80 g, 30 m/min, silica gel, UV 254) to give the product (7.40 g, 81%). ESI-MS m/z calcd for [C₁₃H₁₆N₄O₇][M+NH₄]⁺: 358.1; found: 358.1. ¹H NMR (400 MHz, CDCl₃) δ 5.52-5.41 (m, 2H), 4.25 (d, J=10.0 Hz, 1H), 4.16-4.08 (m, 1H), 4.07-4.00 (m, 1H), 3.92-3.83 (m, 1H), 3.59 (dd, J=10.0, 3.2 Hz, 1H), 2.19 (s, 6H), 2.06 (s, 3H).

### Methyl 2,6-anhydro-4-azido-5,7-O-benzylidene-4-deoxy-D-glycero-L-manno-heptonate

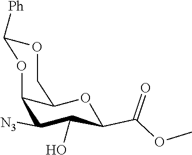

To a solution of 2,4,6-tri-O-acetyl-3-azido-3-deoxy-β-D-galactopyranosyl cyanide (7.40 g, 21.7 mmol) in MeOH (100 mL) at 0° C. acetyl chloride (8.535 g, 109 mmol) was added and the mixture was stirred overnight at 65° C. The mixture was concentrated, and the residue was dissolved in DMF (10 mL). Benzaldehyde dimethylacetal (9.918 g, 65.2 mmol) followed by D(+)-10-camphorsulfonic acid (1.009 g, 4.34 mmol) were added and the mixture was stirred 3 h at 50° C. under reduced pressure. The mixture was poured into water (30 mL) and extracted with EtOAc (3×30 mL). The combined organic phases were washed with brine, concentrated and purified by column chromatography (PE/EtOAc=5/1-1/1, Silica-CS 40 g, 30 mL/min, silica gel, UV 254) to give the product (4.00 g, 55%). ESI-MS m/z calcd for [C₁₅H₁₇N₃O₆] [M+NH₄]⁺: 353.1; found: 353.1. ¹H NMR (400 MHz, CDCl₃) δ 7.55-7.45 (m, 2H), 7.42-7.30 (m, 3H), 5.58 (s, 1H), 4.45-4.34 (m, 2H), 4.26 (d, J=2.8 Hz, 1H), 4.06 (dd, J=12.8, 2.0 Hz, 1H), 3.89 (d, J=9.6 Hz, 1H), 3.84 (s, 3H), 3.52 (d, J=1.2 Hz, 1H), 3.50-3.46 (m, 1H), 3.41 (dd, J=10.4, 3.6 Hz, 1H).

### 2,6-Anhydro-4-azido-5,7-O-benzylidene-4-deoxy-D-glycero-L-manno-heptonic acid

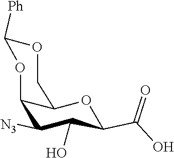

To a solution of methyl 2,6-anhydro-4-azido-5,7-O-benzylidene-4-deoxy-D-glycero-L-manno-heptonate (1.10 g, 3.28 mmol) in THF (50 mL) and water (10 mL) lithium hydroxide monohydrate (688 mg, 16.4 mmol) was added and the mixture was stirred overnight at rt. The mixture was concentrated, and the residue was diluted with water (50 mL). The pH was adjusted to 2-3 using HCl (1 M) and the solids were filtered off, washed with water and dried to give the product (880 mg, 84%). ESI-MS m/z calcd for [C₁₄H₁₅N₃O₆] [M+NH₄]⁺: 339.1; found: 339.3. ¹H NMR (400 MHz, DMSO-d₆) δ 12.93 (s, 1H), 7.68-7.20 (m, 5H), 5.65 (s, 1H), 4.34 (d, J=3.2 Hz, 1H), 4.14-4.01 (m, 2H), 3.94 (t, J=9.6 Hz, 1H), 3.76 (d, J=9.2 Hz, 1H), 3.62 (s, 1H), 3.51 (dd, J=10.0, 3.2 Hz, 1H).

### 2,6-Anhydro-4-azido-5,7-O-benzylidene-4-deoxy-1-C—(N-methoxy-N-methyl)-aldehydo-D-glycero-L-manno-heptose

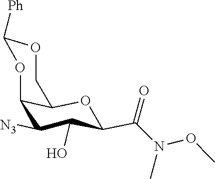

To a stirred solution of 2,6-anhydro-4-azido-5,7-O-benzylidene-4-deoxy-D-glycero-L-manno-heptonic acid (880 mg, 2.74 mmol) in DMF (15.0 mL), N,O-dimethylhydroxylamine hydrochloride (321 mg, 3.29 mmol), HATU (1562 mg, 4.11 mmol) and DIPEA (1.41 mL, 8.22 mmol) were added sequentially and the mixture was stirred 1 h at rt. The mixture was extracted with EtOAc (2×100 mL) and washed with brine (50 mL). The organic phases were evaporated and purified by column chromatography (PE/EtOAc=0˜100%, Silica-CS 20 g, 30 m/min, silica gel, UV 254) to give the product (990 mg, 99%). ESI-MS m/z calcd for [C₁₆H₂₀N₄O₆] [M+H]⁺: 365.1; found: 365.3. ¹H NMR (400 MHz, Chloroform-d) δ 7.48-7.40 (m, 2H), 7.34-7.26 (m, 3H), 5.53 (d, J=1.6 Hz, 1H), 4.39 (t, J=9.6 Hz, 1H), 4.27-4.17 (m, 3H), 4.11-4.02 (m, 1H), 3.72 (d, J=1.6 Hz, 3H), 3.52 (s, 1H), 3.43-3.36 (m, 1H), 2.81-2.67 (m, 3H).

### 2,6-Anhydro-4-azido-5,7-O-benzylidene-4-deoxy-3-O-methyl-1-C—(N-methoxy-N-methyl)-aldehydo-D-glycero-L-manno-heptose

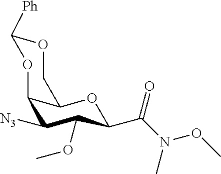

To a solution of 2,6-anhydro-4-azido-5,7-O-benzylidene-4-deoxy-1-C—(N-methoxy-N-methyl)-aldehydo-D-glycero-L-manno-heptose (150 mg, 0.41 mmol) and iodomethane (0.12 mL, 1.65 mmol) in DMF (4.0 mL) silver(I) oxide (191 mg, 0.82 mmol) was added and the mixture was stirred 16 h at rt. The mixture was poured into water (20 mL) and extracted with EtOAc (2×20 mL). The combined organic phases were washed with brine (20 mL), dried over Na₂SO₄, filtered and concentrated. The residue was purified by column chromatography (PE/EtOAc=10/1˜1/1, Silica-CS 4 g, 10 mL/min, silica gel, UV 254) to afford the product (150 mg, 96%). ESI-MS m/z calcd for [C₁₇H₂₂N₄O₆][M+H]⁺: 379.2; found: 379.2. ¹H NMR (400 MHz, Chloroform-d) δ 7.56-7.48 (m, 2H), 7.43-7.30 (m, 3H), 5.57 (s, 1H), 4.37-3.98 (m, 5H), 3.77 (s, 3H), 3.56 (s, 3H), 3.52-3.42 (m, 2H), 3.27 (s, 3H).

### 3,7-Anhydro-5-azido-6,8-O-benzylidene-5-deoxy-4-O-methyl-D-glycero-L-manno-2-octulose

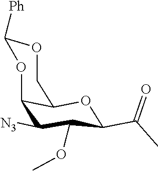

To a solution of 2,6-anhydro-4-azido-5,7-O-benzylidene-4-deoxy-3-O-methyl-1-C—(N-methoxy-N-methyl)-aldehydo-D-glycero-L-manno-heptose (1.0 g, 2.64 mmol) in THF (15 mL) methylmagnesium bromide solution (1.8 mL, 3M in diethyl ether, 1.8 mL) was added dropwise at 0° C. and the mixture was stirred 30 min at 0° C. The reaction was quenched with saturated NH₄Cl and extracted with EtOAc (30 mL). The organic phase was concentrated and purified by column chromatography (PE/EtOAc=0˜60%, Silica-CS 20 g, 30 m/min, silica gel, UV 254) to afford the product (540 mg, 61%). ESI-MS m/z calcd for [C₁₆H₁₉N₃O₅] [M+NH₄]⁺: 351.1; found: 351.0. ¹H NMR (400 MHz, Chloroform-d) δ 7.62-7.46 (m, 2H), 7.45-7.30 (m, 3H), 5.58 (s, 1H), 4.32 (dd, J=12.4, 1.6 Hz, 1H), 4.27 (d, J=2.8 Hz, 1H), 4.06 (dd, J=12.4, 1.6 Hz, 1H), 3.82 (t, J=9.2 Hz, 1H), 3.74 (d, J=9.2 Hz, 1H), 3.52 (s, 3H), 3.48 (d, J=0.8 Hz, 1H), 3.45 (dd, J=9.6, 3.2 Hz, 1H), 2.36 (s, 3H).

### 1-[5-(3-Azido-3-deoxy-2-O-methyl-β-D-galactopyranosyl)-1H-1,2-pyrazol-1-yl]-5-chloro-2-(trifluoromethyl)benzene

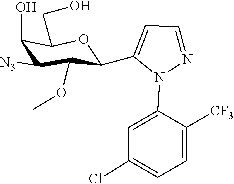

A solution of 3,7-anhydro-5-azido-6,8-O-benzylidene-5-deoxy-4-O-methyl-D-glycero-L-manno-2-octulose (540 mg, 1.62 mmol) in N,N-dimethylformamide dimethyl acetal (5.0 mL) was stirred 16 h at 80° C. The mixture was concentrated to dryness and the residue was dissolved in EtOH (10 mL). [5-Chloro-2-(trifluoromethyl)phenyl]hydrazine (469 mg, 2.23 mmol) and concentrated HCl (0.2 mL) were added and the mixture was stirred 2 h at 80° C. The mixture was concentrated and purified by column chromatography (PE/EA=10/1˜1/2, Silica-CS 12 g, 20 m/min, silica gel, UV 254) to afford the product (300 mg, 38%). ESI-MS m/z calcd for [C₁₇H₁₇ClF₃N₅O₄] [M+H]⁺: 448.1; found: 447.8. ¹H NMR (400 MHz, Methanol-d₄) δ 7.90 (d, J=8.4 Hz, 1H), 7.79 (d, J=8.4 Hz, 1H), 7.74 (d, J=2.0 Hz, 1H), 7.73 (br s, 1H), 6.76 (d, J=2.0 Hz, 1H), 4.02-3.54 (m, 5H), 3.45-3.27 (m, 5H).

### Intermediate 3

### Trimethyl-[2-(5-methylthiazol-2-yl)ethynyl]silane

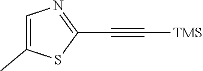

To a solution of 2-bromo-5-methylthiazole (0.55 g, 3.09 mmol) in THF (15 mL) Et₃N (1.72 mL, 12.4 mmol), bis(triphenylphosphine)palladium(II) chloride (108 mg, 0.15 mmol), ethynyl(trimethyl)silane (607 mg, 6.18 mmol) and CuI (58.8 mg, 0.31 mmol) were added and the mixture was stirred overnight at 50° C. The mixture was concentrated, the residue was dissolved in EtOAc and washed with water and brine. The organic phase was dried over Na₂SO₄, concentrated, and purified by column chromatography (PE/EA=1/0˜10/1, Silica-CS 12 g, 20 m/min, silica gel, UV 254) to afford the product (160 mg, 27%). ¹H NMR (400 MHz, Chloroform-d) δ 7.44 (d, J=0.8 Hz, 1H), 2.48 (d, J=1.2 Hz, 3H), 0.27 (s, 9H).

### Intermediate 4

### 4-Methyl-2-((trimethylsilyl)ethynyl)thiazole

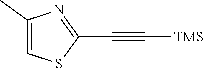

To a solution of 2-bromo-4-methylthiazole (300 mg, 1.68 mmol) in THF (5 mL) ethynyl(trimethyl)silane (248 mg, 2.53 mmol), CuI (16 mg, 0.084 mmol) and bis(triphenylphosphine)palladium(II) chloride (59.1 mg, 0.084 mmol) and Et₃N (0.47 mL, 3.37 mmol) were added and the mixture was stirred 2 h at 60° C. under a nitrogen atmosphere. The mixture was diluted with water (20 mL) and extracted with EA (3×20 mL). The organic phases were washed with brine, dried over MgSO₄, concentrated, and purified by column chromatography (PE/EA=20/1˜10/1, Silica-CS 40 g, 50 m/min, silica gel, UV 254) to afford the product (225 mg, 68%). ¹H NMR (400 MHz, CDCl₃) δ 6.87 (s, 1H), 2.45 (s, 3H), 0.26 (s, 9H).

### Intermediate 5

### 2,6-Anhydro-4-azido-5,7-O-benzylidene-4-deoxy-3-O-(methoxymethyl)-1-C—(N-methoxy-N-methyl)-aldehydo-D-glycero-L-manno-heptose

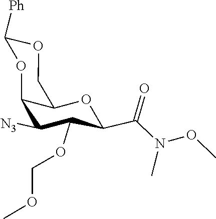

To a solution of 2,6-anhydro-4-azido-5,7-O-benzylidene-4-deoxy-1-C—(N-methoxy-N-methyl)-aldehydo-D-glycero-L-manno-heptose (790 mg, 2.17 mmol) and bromo(methoxy)methane (2.71 g, 21.7 mmol) in DMF (10.0 mL) silver(I) oxide (1005 mg, 4.34 mmol) and NaI (650 mg, 4.34 mmol) were added and the mixture was stirred 60 h at rt. The mixture was concentrated and purified by column chromatography (PE/EtOAc=1/0˜0/1, Silica-CS 20 g, 30 mL/min, silica gel, UV 254) to afford the product (400 mg, 45%). ESI-MS m/z calcd for [C₁₈H₂₄N₄O₇] [M+H]⁺: 409.2; found: 409.3. ¹H NMR (400 MHz, Chloroform-d) δ 7.49-7.43 (m, 2H), 7.33-7.25 (m, 3H), 5.54 (s, 1H), 4.88-4.79 (m, 1H), 4.77-4.70 (m, 1H), 4.68-4.63 (m, 1H), 4.49 (t, J=9.6 Hz, 1H), 4.31-4.15 (m, 3H), 4.00 (dd, J=12.4, 1.6 Hz, 1H), 3.68 (s, 3H), 3.35 (s, 3H), 3.24 (dd, J=10.0, 3.2 Hz, 1H), 3.16 (s, 3H).

### 3,7-Anhydro-5-azido-6,8-O-benzylidene-5-deoxy-4-O-(methoxymethyl)-D-glycero-L-manno-2-octulose

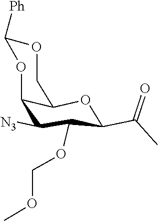

To a cooled (0° C.) solution of 2,6-anhydro-4-azido-5,7-O-benzylidene-4-deoxy-3-O-(methoxymethyl)-1-C—(N-methoxy-N-methyl)-aldehydo-D-glycero-L-manno-heptose (400 mg, 0.98 mmol) in THF (5.0 mL) methylmagnesium bromide (0.653 mL, 3 M in diethyl ether, 1.96 mmol) was added fropwise and the mixture was stirred 30 h at 0° C. The mixture was quenched by addition of saturated NH₄Cl and extracted with EtOAc (30 mL). The organic phase was dried, evaporated and purified by column chromatography (PE/EtOAc=0˜60%, Silica-CS 20 g, 30 mL/min, silica gel, UV 254) to afford the product (310 mg, 87%). ESI-MS m/z calcd for [C₁₇H₂₁N₃O₆] [M+H]⁺: 381.1; found: 381.3. ¹H NMR (400 MHz, Chloroform-d) δ 7.51-7.42 (m, 2H), 7.37-7.26 (m, 3H), 5.53 (s, 1H), 4.71 (d, J=6.8 Hz, 1H), 4.57 (d, J=6.8 Hz, 1H), 4.32-4.20 (m, 2H), 4.13-3.93 (m, 2H), 3.67 (d, J=9.6 Hz, 1H), 3.42 (d, J=1.2 Hz, 1H), 3.35-3.31 (m, 1H), 3.28 (s, 3H), 2.28 (s, 3H).

### 1-[5-(3-Azido-3-deoxy-β-D-galactopyranosyl)-1H-1,2-pyrazol-1-yl]-5-chloro-2-(trifluoromethyl)benzene

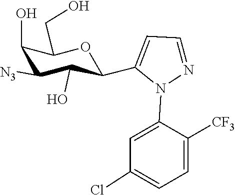

A solution of 3,7-anhydro-5-azido-6,8-O-benzylidene-5-deoxy-4-O-(methoxymethyl)-D-glycero-L-manno-2-octulose (530 mg, 1.46 mmol) in N,N-dimethylformamide dimethyl acetal (6 mL) was stirred 16 h at 80° C. The mixture was concentrated to dryness and the residue was dissolved in EtOH (10.0 mL). [5-Chloro-2-(trifluoromethyl)phenyl]hydrazine (461 mg, 2.19 mmol) and concentrated HCl (0.2 mL) were added and the mixture was stirred 2 h at 80° C. The mixture was concentrated and partitioned between DCM (50 mL) saturated aq NaHCO₃ (50 mL). The organic phase was extracted with saturated aq NaHCO₃ (2×50 mL). The combined aqueous phases were concentrated and purified by column chromatography (PE/EtOAc=10/1˜0/1, Silica-CS 12 g, 20 m/min, silica gel, UV 254) to afford the product (110 mg, 17%). ESI-MS m/z calcd for [C₁₆H₁₅ClF₃N₅O₄] [M+H]⁺: 434.1; found: 434.2. ¹H NMR (400 MHz, Methanol-d₄) δ 7.89 (d, J=8.4 Hz, 1H), 7.78 (dd, J=8.4, 1.2 Hz, 1H), 7.72-7.70 (m, 2H), 6.68 (d, J=2.0 Hz, 1H), 4.28-4.05 (m, 1H), 4.01-3.91 (m, 2H), 3.63-3.52 (m, 2H), 3.35 (t, J=6.0 Hz, 1H), 3.30-3.26 (m, 1H).

### Intermediate 6

### tert-Butyl N-tert-butoxycarbonyl-N-[4-(2-trimethylsilylethynyl)thiazol-2-yl]carbamate

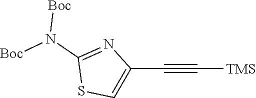 [SMILES: CC(C)(C)OC(=O)N(C(=O)OC(C)(C)C)c1nc(C#C[Si](C)(C)C)cs1]

To a solution of 4-(2-trimethylsilylethynyl)thiazol-2-amine (2.00 g, 10.2 mmol) in DCM (50 mL) di-tert-butyl dicarbonate (4.45 g, 20.4 mmol), Et₃N (5.68 mL, 40.8 mmol) and 4-(dimethylamino)pyridine (12.4 mg, 1.02 mmol) were added and the mixture was stirred 5 h at rt. Water (100 mL) was added, and the mixture was extracted with diethyl ether (3×100 mL). The combined organic layers were washed with brine (100 mL), dried over Na₂SO₄, evaporated, and purified by column chromatography (PE/EtOAc=1/0˜10/1, Silica-CS 40 g, 40 mL/min, silica gel, UV 254) to give the product (3.60 g, 89%). ESI-MS m/z calcd for [C₁₈H₂₈N₂O₄SSi] [M+H]⁺: 397.2; found: 397.3. ¹H NMR (400 MHz, CDCl₃) δ 7.25-7.18 (m, 1H), 1.45 (d, J=2.8 Hz, 18H), 0.18 (d, J=2.8 Hz, 9H).

### 1-{5-{3-{4-[2-(Di-tert-butoxycarbonylamino)thiazol-4-yl]-1H-1,2,3-triazol-1-yl}-3-deoxy-β-D-galactopyranosyl}-1H-1,2-pyrazol-1-yl}-5-chloro-2-(trifluoromethyl)benzene

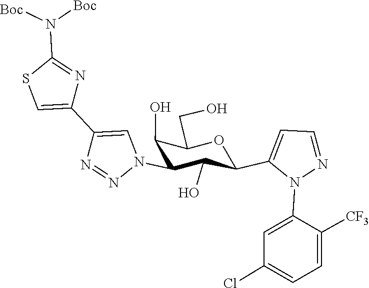

To a solution of 1-[5-(3-azido-3-deoxy-β-D-galactopyranosyl)-1H-1,2-pyrazol-1-yl]-5-chloro-2-(trifluoromethyl) (60 mg, 0.14 mmol) in DMF (3 mL) tert-butyl N-tert-butoxycarbonyl-N-[4-(2-trimethylsilylethynyl)thiazol-2-yl]carbamate (110 mg, 0.28 mmol), copper(II) sulfate pentahydrate (35 mg, 0.14 mmol) and (+)-sodium L-ascorbate (28 mg, 0.14 mmol) were added and the mixture was stirred overnight at rt. The mixture was concentrated and purified by column chromatography (PE/EtOAc=10/1˜1/1, Silica-CS 4 g, 10 mL/min, silica gel, UV 254) to give the product (66 mg, 63%). ESI-MS m/z calcd for [C₃₁H₃₅ClF₃N₇O₈S] [M+H]⁺: 758.2; found: 758.1. ¹H NMR (400 MHz, Chloroform-d) δ 8.12 (s, 1H), 7.77 (d, J=8.4 Hz, 1H), 7.72 (d, J=1.6 Hz, 1H), 7.66-7.54 (m, 3H), 6.60 (d, J=1.6 Hz, 1H), 4.70-4.57 (m, 1H), 4.51 (d, J=9.6 Hz, 1H), 4.41 (s, 1H), 4.30-4.18 (m, 1H), 3.91-3.76 (m, 2H), 3.58 (t, J=4.8 Hz, 1H), 1.53 (s, 18H).

### Intermediate 8

### 2-(3-Chloropyrazol-1-yl)ethynyl(triisopropyl)silane

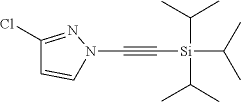

A solution of 3-chloro-1H-pyrazole (150 mg, 1.46 mmol), CuI (14 mg, 0.073 mmol), cesium carbonate (572 mg, 1.76 mmol) and 2-bromoethynyl(triisopropyl)silane (765 mg, 2.93 mmol) in 1,4-dioxane (2 mL) and PEG400 (400 mg) was stirred 4 h at 70° C. The mixture was filtered through a plug of celite, concentrated and purified by chromatography (SiO₂, PE/EtOAc) to afford the product (68 mg, 16%). ESI-MS m/z calcd for [C₁₄H₂₃ClN₂Si] [M+H]⁺: 283.1; found: 283.1.

### Intermediate 9

### 3,7-Anhydro-6,8-O-benzylidene-5-[4-(4-chlorothiazol-2-yl)-1H-1,2,3-triazol-1-yl]-5-deoxy-4-O-(methoxymethyl)-D-glycero-L-manno-2-octulose

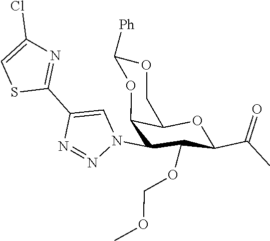

To a solution of 3,7-anhydro-5-azido-6,8-O-benzylidene-5-deoxy-4-O-(methoxymethyl)-D-glycero-L-manno-2-octulose (310 mg, 0.85 mmol) and 2-(4-chlorothiazol-2-yl)ethynyltrimethylsilane (221 mg, 1.02 mmol) in DMF (4.0 mL) (+)-sodium L-ascorbate (254 mg, 1.28 mmol) and copper(II) sulfate pentahydrate (107 mg, 0.43 mmol) were added and the mixture was stirred 4 h at rt. The mixture was concentrated and purified by column chromatography (PE/EtOAc=2/1-1/2, Silica-CS 20 g, 30 mL/min, silica gel, UV 254) to afford the product (330 mg, 76%). ESI-MS m/z calcd for [C₂₂H₂₃ClN₄O₆S] [M+H]⁺: 507.1; found: 507.1. ¹H NMR (400 MHz, Chloroform-d) δ 8.32 (s, 1H), 7.43-7.30 (m, 5H), 7.02 (s, 1H), 5.42 (s, 1H), 5.06 (dd, J=10.4, 3.2 Hz, 1H), 4.40-4.25 (m, 4H), 4.12 (d, J=7.2 Hz, 1H), 4.07-3.99 (m, 1H), 3.91 (d, J=9.2 Hz, 1H), 3.73-3.64 (m, 1H), 2.72 (s, 3H), 2.33 (s, 3H).

### Intermediate 10

### 2-(5-Chlorothiazol-2-yl)ethynyl(trimethyl)silane

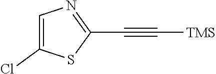 [SMILES: C[Si](C)(C)C#Cc1ncc(Cl)s1]

To a solution of 2-bromo-5-chlorothiazole (1.0 g, 5.0 mmol) in THF (30.0 mL) Pd(PPh₃)₂Cl₂ (177 mg, 0.25 mmol), CuI (48 mg, 0.25 mmol), (trimethyl)silylacetylene (1.49 g, 15 mmol) and Et₃N (2.1 mL, 15 mmol) were added and the mixture was stirred 4 h at 50° C. The mixture was concentrated, water (50 mL) was added, and the solution was extracted with DCM (2×50 mL). The combined organic phases were washed with brine, dried over Na₂SO₄, evaporated, and purified by column chromatography (EtOAc/PE=0˜1/10, Silica-CS 40 g, 40 mL/min, silica gel, UV 254) to afford the product (565 mg, 52%). ¹H NMR (400 MHz, Chloroform-d) δ 7.08 (s, 1H), 0.27 (s, 9H).

### 3,7-Anhydro-6,8-O-benzylidene-5-[4-(5-chlorothiazol-2-yl)-1H-1,2,3-triazol-1-yl]-5-deoxy-4-O-(methoxymethyl)-D-glycero-L-manno-2-octulose

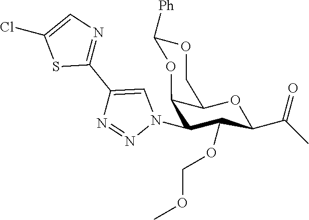

To a solution of 3,7-anhydro-5-azido-6,8-O-benzylidene-5-deoxy-4-O-(methoxymethyl)-D-glycero-L-manno-2-octulose (130 mg, 0.26 mmol) and 2-(5-chlorothiazol-2-yl)ethynyltrimethylsilane (83 mg, 0.39 mmol) in DMF (3 mL) (+)-sodium L-ascorbate (50.8 mg, 0.26 mmol) and copper(II) sulfate pentahydrate (64 mg, 0.26 mmol) were added and the mixture was stirred overnight at rt. The mixture was poured into water (50 mL) and extracted with EtOAc (2×80 mL). The combined organic phases were washed with brine (50 mL), dried over Na₂SO₄, concentrated, and purified by column chromatography (DCM/EtOAc=1/0˜2/1, Silica-CS 4 g, 10 mL/min, silica gel, UV 254) to afford the product (112 mg, 86%). ESI-MS m/z calcd for [C₂₂H₂₃ClN₄O₆S] [M+H]⁺: 507.1; found: 507.0. ¹H NMR (400 MHz, Chloroform-d) δ 8.40 (s, 1H), 7.46-7.40 (m, 5H), 7.09 (s, 1H), 5.49 (s, 1H), 5.13 (dd, J=10.4, 3.2 Hz, 1H), 4.45-4.37 (m, 4H), 4.19 (d, J=7.2, 1H), 4.09 (dd, J=12.4, 1.6 Hz, 1H), 3.98 (d, J=9.2 Hz, 1H), 3.75 (d, J=0.4 Hz, 1H), 2.79 (s, 3H), 2.40 (s, 3H).

### Intermediate 12

### 3,7-Anhydro-6,8-O-benzylidene-5-[4-(5-chlorothiazol-2-yl)-1H-1,2,3-triazol-1-yl]-5-deoxy-4-O-methyl-D-glycero-L-manno-2-octulose

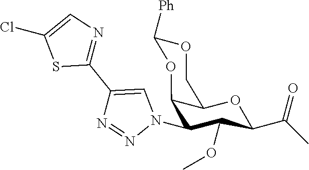

To a solution of 3,7-anhydro-5-azido-6,8-O-benzylidene-5-deoxy-4-O-methyl-D-glycero-L-manno-2-octulose (110 mg, 0.33 mmol) in DMF (2.5 mL) 2-(5-chlorothiazol-2-yl)ethynyltrimethylsilane (59.8 mg, 0.28 mmol), copper(II) sulfate pentahydrate (41 mg, 0.17 mmol) and (+)-sodium L-ascorbate (33 mg, 0.17 mmol) were added and the mixture was stirred 3 h at rt. The mixture was concentrated and purified by column chromatography (DCM/EtOAc=1/0˜2/1, Silica-CS 20 g, 25 mL/min, silica gel, UV 254) to afford the product (120 mg, 76%). ESI-MS m/z calcd for [C₂₁H₂₁ClN₄O₅S] [M+H]⁺: 477.1; found: 477.0 ¹H NMR (400 MHz, Chloroform-d) δ 8.40 (s, 1H), 7.47-7.40 (m, 5H), 7.11 (s, 1H), 5.50 (s, 1H), 5.07 (dd, J=10.4, 3.2 Hz, 1H), 4.42-4.38 (m, 2H), 4.17-4.07 (m, 2H), 3.95 (d, J=9.2 Hz, 1H), 3.73 (s, 1H), 3.06 (s, 3H), 2.40 (s, 3H).

### Intermediate 13

### 3,7-Anhydro-6,8-O-benzylidene-5-[4-(4-chlorothiazol-2-yl)-1H-1,2,3-triazol-1-yl]-5-deoxy-4-O-methyl-D-glycero-L-manno-2-octulose

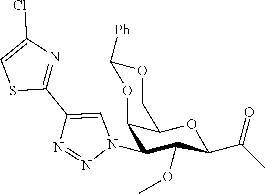

To a solution of 3,7-anhydro-5-azido-6,8-O-benzylidene-5-deoxy-4-O-methyl-D-glycero-L-manno-2-octulose (110 mg, 0.33 mmol) in DMF (2.5 mL) 2-(4-chlorothiazol-2-yl)ethynyltrimethylsilane (142 mg, 0.66 mmol), copper(II) sulfate pentahydrate (41 mg, 0.17 mmol) and (+)-sodium L-ascorbate (33 mg, 0.17 mmol) were added and the mixture was stirred 3 h at rt. The mixture was concentrated and purified by column chromatography (DCM/EtOAc=1/0˜2/1, Silica-CS 20 g, 25 mL/min, silica gel, UV 254) to afford the product (140 mg, 89%). ESI-MS m/z calcd for [C₂₁H₂₁ClN₄O₅S] [M+H]⁺: 477.1; found: 477.0 ¹H NMR (400 MHz, Chloroform-d) δ 8.34 (s, 1H), 7.46-7.40 (m, 5H), 7.11 (s, 1H), 5.50 (s, 1H), 5.07 (dd, J=10.4, 2.8 Hz, 1H), 4.42-4.38 (m, 2H), 4.17-4.07 (m, 2H), 3.95 (d, J=9.2 Hz, 1H), 3.73 (d, J=1.2 Hz, 1H), 3.06 (s, 3H), 2.40 (s, 3H).

### Intermediate 14

### 3,7-Anhydro-6,8-O-benzylidene-5-deoxy-4-O-methyl-5-[4-(thiazol-2-yl)-1H-1,2,3-triazol-1-yl]-D-glycero-L-manno-2-octulose

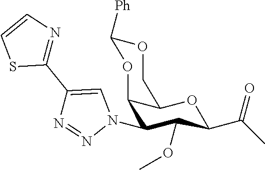

To a solution of 3,7-anhydro-5-azido-6,8-O-benzylidene-5-deoxy-4-O-methyl-D-glycero-L-manno-2-octulose (192 mg, 0.58 mmol) in DMF (3 mL) trimethyl(2-thiazol-2-ylethynyl)silane (209 mg, 1.15 mmol), copper(II) sulfate pentahydrate (144 mg, 0.58 mmol) and (+)-sodium L-ascorbate (114 mg, 0.58 mmol) were added and the mixture was stirred overnight at rt. The mixture was poured into water (50 mL) and extracted with EtOAc (2×80 mL). The combined organic phases were washed with brine (50 mL), dried over Na₂SO₄, concentrated, and purified by column chromatography (PE/EtOAc=1/0˜1/1, Silica-CS 4 g, 10 m/min, silica gel, UV 254) to afford the product (181 mg, 71%). ESI-MS m/z calcd for [C₂₁H₂₂N₄O₅S] [M+H]⁺: 443.1; found: 443.0 ¹H NMR (400 MHz, Chloroform-d) δ 8.47 (s, 1H), 7.85 (br s, 1H), 7.48-7.38 (m, 6H), 5.50 (s, 1H), 5.08 (dd, J=10.4, 3.2 Hz, 1H), 4.44 (d, J=2.8 Hz, 1H), 4.40 (dd, J=12.8, 1.2 Hz, 1H), 4.19 (t, J=9.6 Hz, 1H), 4.11-4.08 (m, 1H), 3.96 (d, J=9.2 Hz, 1H), 3.74 (s, 1H), 3.07 (s, 3H), 2.40 (s, 3H).

### Intermediate 17

### 1-[5-(3-Azido-4,6-O-benzylidene-3-deoxy-2-O-methyl-β-D-galactopyranosyl)-3-ethoxycarbonyl-1H-1,2-pyrazol-1-yl]-5-chloro-2-(trifluoromethyl)benzene

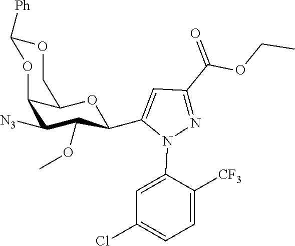

### 1-[5-(3-Azido-3-deoxy-2-O-methyl-β-D-galactopyranosyl)-3-ethoxycarbonyl-1H-1,2-pyrazol-1-yl]-5-chloro-2-(trifluoromethyl)benzene

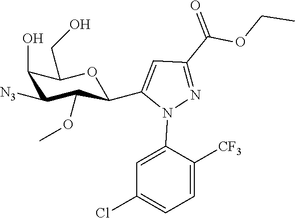

To a solution of 3,7-anhydro-5-azido-6,8-O-benzylidene-5-deoxy-4-O-methyl-D-glycero-L-manno-2-octulose (1.14 g, 7.80 mmol) in dry THF (10 mL) diethyl oxalate (1.14 g, 7.80 mmol) was added. The mixture was cooled to −78° C. and lithium diisopropylamide (3 mL, 2M in THF) was added. The mixture was slowly warmed to rt and stirred for 2 h. The mixture was concentrated, and the residue was dissolved in EtOH (30 mL). [5-Chloro-2-(trifluoromethyl)phenyl]hydrazine (1.31 g, 6.24 mmol) was added and the mixture was stirred 2 h at rt. The mixture was concentrated, the residue was dissolved in acetic acid (20 mL) and the mixture was stirred 2 h at 100° C. The mixture was concentrated and purified by column chromatography (EtOAc/PE=0/1˜1/1, Silica-CS 40 g, 40 mL/min, silica gel, UV 254) to afford both 1-[5-(3-azido-4,6-O-benzylidene-3-deoxy-2-O-methyl-β-D-galactopyranosyl)-3-ethoxycarbonyl-1H-1,2-pyrazol-1-yl]-5-chloro-2-(trifluoromethyl)benzene (640 mg, 27%) and 1-[5-(3-azido-3-deoxy-2-O-methyl-β-D-galactopyranosyl)-3-ethoxycarbonyl-1H-1,2-pyrazol-1-yl]-5-chloro-2-(trifluoromethyl)benzene (560 mg, 28%).

### 1-[5-(3-Azido-4,6-O-benzylidene-3-deoxy-2-O-methyl-β-D-galactopyranosyl)-3-ethoxycarbonyl-1H-1,2-pyrazol-1-yl]-5-chloro-2-(trifluoromethyl)benzene

ESI-MS m/z calcd for [C₂₇H₂₅ClF₃N₅O₆] [M+H]⁺: 608.1; found: 608.3. ¹H NMR (400 MHz, Chloroform-d) δ 7.68-7.35 (m, 8H), 7.17-7.12 (m, 1H), 5.54 (s, 1H), 4.46-4.40 (m, 2H), 4.24-4.15 (m, 2H), 4.03-3.62 (m, 3H), 3.46-3.27 (m, 5H), 1.40 (t, J=7.2 Hz, 3H).

### 1-[5-(3-Azido-3-deoxy-2-O-methyl-β-D-galactopyranosyl)-3-ethoxycarbonyl-1H-1,2-pyrazol-1-yl]-5-chloro-2-(trifluoromethyl)benzene

ESI-MS m/z calcd for [C₂₀H₂₁ClF₃N₅O₆] [M+H]⁺: 520.1; found: 520.2. ¹H NMR (400 MHz, Methanol-d₄) δ 7.93 (d, J=8.8 Hz, 1H), 7.89-7.61 (m, 2H), 7.21 (s, 1H), 4.39 (q, J=7.2 Hz, 2H), 4.02-3.93 (m, 2H), 3.77-3.55 (m, 3H), 3.45-3.35 (m, 5H), 1.39 (t, J=7.2 Hz, 3H).

### Intermediate 20

### 4,8-Anhydro-7,9-O-benzylidene-6-[4-(4-chlorothiazol-2-yl)-1H-1,2,3-triazol-1-yl]-6-deoxy-5-O-methyl-1-N,N-dimethylamino-D-glycero-L-manno-non-1-en-3-ulose

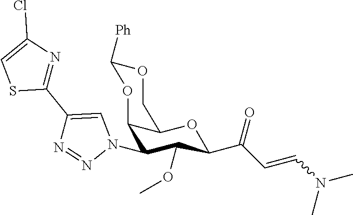

A solution of 3,7-anhydro-6,8-O-benzylidene-5-[4-(4-chlorothiazol-2-yl)-1H-1,2,3-triazol-1-yl]-5-deoxy-4-O-methyl-D-glycero-L-manno-2-octulose (90 mg, 0.20 mmol) dissolved in N,N-dimethylformamide dimethyl acetal (2 mL) was stirred 3 h at 90° C. The mixture was concentrated to dryness and the residue was purified by column chromatography (PE/EtOAc=1/1-0/1, Silica-CS 12 g, 20 mL/min, silica gel, UV 254) to afford the product (82 mg, 77%). ESI-MS m/z calcd for [C₂₄H₂₆ClN₅O₅S] [M+H]⁺: 532.1; found: 532.0. ¹H NMR (400 MHz, Methanol-d₄) δ 8.38 (s, 1H), 7.95 (br s, 1H), 7.48-7.38 (m, 5H), 7.10 (s, 1H), 5.51-5.45 (m, 2H), 5.07 (dd, J=10.8, 3.2 Hz, 1H), 4.43-4.38 (m, 2H), 4.23 (t, J=7.2, 1H), 4.11-3.95 (m, 2H), 3.70 (s, 1H), 3.14-3.05 (m, 6H), 2.92 (s, 3H).

### Intermediate 21

### 4,5-Dichloro-2-(trifluoromethyl)aniline

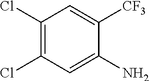 [SMILES: Nc1cc(Cl)c(Cl)cc1C(F)(F)F]

To a solution of 1,2-dichloro-4-nitro-5-(trifluoromethyl)benzene (1.10 g, 4.23 mmol) in acetic acid (20 mL) iron (709 mg, 12.7 mmol) was added and the mixture was stirred 4 h at 80° C. The mixture was filtered, concentrated, and purified by column chromatography (EtOAc/PE=1/10-2/1, Silica-CS 40 g, 50 mL/min, silica gel, UV 254) to afford the product (833 mg, 86%). ¹H NMR (400 MHz, Chloroform-d) δ 7.48 (s, 1H), 6.86 (s, 1H), 4.03 (br s, 2H).

### [4,5-Dichloro-2-(trifluoromethyl)phenyl]hydrazine

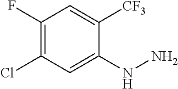 [SMILES: NNc1cc(Cl)c(F)cc1C(F)(F)F]

To a solution of 4,5-chloro-2-(trifluoromethyl)aniline (833 mg, 3.62 mmol) in acetic acid (3 mL) concentrated HCl (3 mL) was added and the mixture was cooled to −5° C. A solution of NaNO₂ (300 mg, 4.35 mmol) in water (2 mL) was added and the mixture was stirred 1 h at 0° C. A solution of tin(II) chloride dihydrate (1.79 g, 7.95 mmol) in concentrated HCl (3 mL) was added slowly at 0° C. The mixture was warmed to rt and stirred 3 h. The mixture was basified to pH=10 by addition of NaOH (1M). The mixture was extracted with EtOAc (2×50 mL). The combined organic phases were dried over Na₂SO₄, concentrated, and purified by column chromatography (DCM/PE=0/1˜4/1, Silica-CS 40 g, 50 mL/min, silica gel, UV 254) to afford the product (356 mg, 40%). ESI-MS m/z calcd for [C₇H₅Cl₂F₃N₂] [M+H]⁺: 245.0; found: 245.1. ¹H NMR (400 MHz, Chloroform-d) δ 7.53 (s, 1H), 7.48 (s, 1H), 5.85 (s, 1H), 3.66 (s, 2H).

### Intermediate 22

### 1-Bromo-4-chloro-5-fluoro-2-nitrobenzene

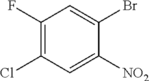 [SMILES: O=[N+]([O-])c1cc(Cl)c(F)cc1Br]

To a cooled (0° C.) solution of 4-bromo-1-chloro-2-fluorobenzene (10.0 g, 47.7 mmol) and NH₄NO₃ (5.73 g, 71.6 mmol) in DCM (350 mL) trifluoroacetic anhydride (46.5 mL, 334 mmol) was added over 15 min. The mixture was stirred 15 min at 0° C. then 2 h at rt. The reaction was quenched with saturated aq NaHCO₃ (560 mL) and the phases were separated. The aqueous phase was extracted with DCM and then EtOAc. The combined organic phases were dried over Na₂SO₄, concentrated, and purified by column chromatography (EtOAc/PE=0/1˜1/7, Silica-CS 80 g, 50 m/min, silica gel, UV 254) to afford the product (4.2 g, 35%). ¹H NMR (400 MHz, DMSO-d₆) δ 8.50 (d, J=6.8 Hz, 1H), 8.21 (d, J=8.8 Hz, 1H).

### 1-Chloro-2-fluoro-5-nitro-4-(trifluoromethyl)benzene

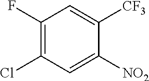 [SMILES: O=[N+]([O-])c1cc(Cl)c(F)cc1C(F)(F)F]

To a solution of 1-bromo-4-chloro-5-fluoro-2-nitrobenzene (4.20 g, 16.5 mmol) in DMF (40 mL) methyl 2,2-difluoro-2-fluorosulfonylacetate (6.64 g, 33.0 mmol) and CuI (3.14 g, 16.5 mmol) were added and the mixture was stirred 2 h at 80° C. The reaction was quenched with saturated aq NaHCO₃ (200 mL) and extracted with EtOAc (2×100 mL). The combined organic phases were washed with brine (100 mL), dried over Na₂SO₄, concentrated, and purified by column chromatography (EtOAc/PE=0/1˜1/7, Silica-CS 80 g, 50 mL/min, silica gel, UV 254) to afford the product (3.7 g, 92%). ¹H NMR (400 MHz, DMSO-d₆) δ 8.65 (d, J=6.4 Hz, 1H), 8.30 (d, J=9.2 Hz, 1H).

### 5-Chloro-4-fluoro-2-(trifluoromethyl)aniline

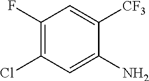 [SMILES: Nc1cc(Cl)c(F)cc1C(F)(F)F]

To a solution of 1-chloro-2-fluoro-5-nitro-4-(trifluoromethyl)benzene (1.80 g, 7.39 mmol) in acetic acid (20 mL) iron (1.24 g, 22.2 mmol) was added and the mixture was stirred 4 h at 80° C. The mixture was filtered, concentrated, and purified by column chromatography (EtOAc/PE=0/1˜1/4, Silica-CS 40 g, 50 m/min, silica gel, UV 254) to afford the product (1.3 g, 82%). ¹H NMR (400 MHz, DMSO-d₆) δ 7.41 (d, J=9.6 Hz, 1H), 7.00 (d, J=6.4 Hz, 1H), 5.75 (s, 2H).

### [5-Chloro-4-fluoro-2-(trifluoromethyl)phenyl]hydrazine

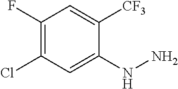 [SMILES: NNc1cc(Cl)c(F)cc1C(F)(F)F]

To a solution of 5-chloro-4-fluoro-2-(trifluoromethyl)aniline (1.30 g, 6.09 mmol) in acetic acid (5 mL) concentrated HCl (5 mL) was added and the mixture was cooled to −5° C. A solution of NaNO₂ (630 mg, 9.13 mmol) in water (2 mL) was added and the mixture was stirred 1 h at 0° C. A solution of tin(II) chloride dihydrate (2.75 g, 12.2 mmol) in concentrated HCl (5 mL) was added slowly at 0° C. The mixture was warmed to rt and stirred 3 h. The mixture was basified to pH=10 by addition of NaOH (1M). The mixture was extracted with EtOAc (2×50 mL). The combined organic phases were dried over Na₂SO₄, concentrated, and purified by column chromatography (DCM/PE=0/1˜4/1, Silica-CS 40 g, 50 mL/min, silica gel, UV 254) to afford the product (433 mg, 31%). ESI-MS m/z calcd for [C₇H₅ClF₄N₂] [M+H]⁺: 229.0; found: 229.1. ¹H NMR (400 MHz, DMSO-d₆) δ 7.51 (d, J=6.8 Hz, 1H), 7.48 (d, J=9.2 Hz, 1H), 6.96 (s, 1H), 4.30 (s, 2H).

### Intermediate 23

### 1,4-Dibromo-2-fluoro-5-nitrobenzene

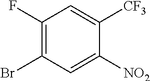 [SMILES: O=[N+]([O-])c1cc(Br)c(F)cc1C(F)(F)F]

To a cooled (0° C.) solution of 1,4-bromo-2-fluorobenzene (3.00 g, 11.8 mmol) and NH₄NO₃ (1.42 g, 17.7 mmol) in DCM (100 mL) trifluoroacetic anhydride (11.5 mL, 82.7 mmol) was added over 15 min. The mixture was stirred 15 min at 0° C. then 2 h at rt. The reaction was quenched with saturated aq NaHCO₃ (560 mL) and the phases were separated. The aqueous phase was extracted with DCM and then EtOAc. The combined organic phases were dried over Na₂SO₄, concentrated, and purified by column chromatography (EtOAc/PE=0/1˜1/7, Silica-CS 80 g, 50 m/min, silica gel, UV 254) to afford the product (2.8 g, 35%). ¹H NMR (400 MHz, Chloroform-d) δ 8.11 (d, J=6.4 Hz, 1H), 7.46 (d, J=7.2 Hz, 1H).

### 1-Bromo-2-fluoro-5-nitro-4-(trifluoromethyl)benzene

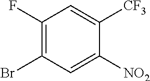 [SMILES: O=[N+]([O-])c1cc(Br)c(F)cc1C(F)(F)F]

To a solution of 1,4-dibromo-2-fluoro-5-nitrobenzene (2.80 g, 9.37 mmol) in DMF (25 mL) methyl 2,2-difluoro-2-fluorosulfonylacetate (3.6 g, 18.7 mmol) and CuI (1.78 g, 9.37 mmol) were added and the mixture was stirred 2 h at 80° C. The reaction was quenched with saturated aq NaHCO₃ (200 mL) and extracted with EtOAc (3×100 mL). The combined organic phases were washed with brine (2×100 mL), dried over Na₂SO₄, concentrated, and purified by column chromatography (EtOAc/PE=0/1˜1/7, Silica-CS 80 g, 50 m/min, silica gel, UV 254) to afford the product (1.85 g, 69%). ¹H NMR (400 MHz, Chloroform-d) δ 8.16 (d, J=6.0 Hz, 1H), 7.52 (d, J=8.0 Hz, 1H).

### 5-Bromo-4-fluoro-2-(trifluoromethyl)aniline

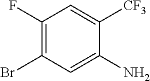 [SMILES: Nc1cc(Br)c(F)cc1C(F)(F)F]

To a solution of 1-bromo-2-fluoro-5-nitro-4-(trifluoromethyl)benzene (2.13 g, 7.40 mmol) in acetic acid (20 mL) iron (1.24 g, 22.2 mmol) was added and the mixture was stirred 4 h at 80° C. The mixture was concentrated and purified by column chromatography (EtOAc/PE=0/1˜1/4, Silica-CS 40 g, 50 m/min, silica gel, UV 254) to afford the product (1.05 g, 55%). ¹H NMR (400 MHz, DMSO-d₆) δ 7.37 (d, J=9.2 Hz, 1H), 7.15 (d, J=6.0 Hz, 1H), 5.74 (br s, 2H).

### [5-Bromo-4-fluoro-2-(trifluoromethyl)phenyl]hydrazine

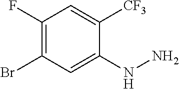 [SMILES: NNc1cc(Br)c(F)cc1C(F)(F)F]

To a solution of 5-bromo-4-fluoro-2-(trifluoromethyl)aniline (1.05 g, 4.07 mmol) in acetic acid (5 mL) concentrated HCl (5 mL) was added and the mixture was cooled to −5° C. A solution of NaNO₂ (421 mg, 6.10 mmol) in water (2 mL) was added and the mixture was stirred 1 h at 0° C. A solution of tin(II) chloride dihydrate (1.84 g, 8.14 mmol) in concentrated HCl (5 mL) was added slowly at 0° C. The mixture was warmed to rt and stirred 3 h. The mixture was basified to pH=10 by addition of NaOH (1M). The mixture was extracted with EtOAc (2×50 mL). The combined organic phases were dried over Na₂SO₄, concentrated, and purified by column chromatography (DCM/MeOH=1/0˜4/1, Silica-CS 40 g, 50 m/min, silica gel, UV 254) to afford the product (405 mg, 37%). ESI-MS m/z calcd for [C₇H₅BrF₄N₂] [M+H]⁺: 273.0; found: 273.0. ¹H NMR (400 MHz, DMSO-d₆) δ 7.66 (d, J=6.0 Hz, 1H), 7.43 (d, J=8.8 Hz, 1H), 6.93 (br s, 1H), 4.30 (br s, 2H).

### Intermediate 24

### 4,8-Anhydro-7,9-O-benzylidene-6-[4-(4-chlorothiazol-2-yl)-1H-1,2,3-triazol-1-yl]-6-deoxy-5-O-(methoxymethyl)-1-N,N-dimethylamino-D-glycero-L-manno-non-1-en-3-ulose

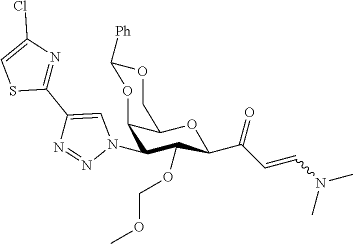

A solution of 3,7-anhydro-6,8-O-benzylidene-5-[4-(4-chlorothiazol-2-yl)-1H-1,2,3-triazol-1-yl]-5-deoxy-4-O-(methoxymethyl)-D-glycero-L-manno-2-octulose (82 mg, 0.16 mmol) dissolved in N,N-dimethylformamide dimethyl acetal (2 mL) was stirred 3 h at 80° C. The mixture was concentrated to dryness and the residue was purified by column chromatography (PE/EtOAc=1/1-0/1, Silica-CS 12 g, 20 m/min, silica gel, UV 254) to afford the product (56 mg, 62%). ESI-MS m/z calcd for [C₂₅H₂₈ClN₅O₆S][M+H]⁺: 562.1; found: 562.2.

### Intermediate 25

### 1-[5-(3-Azido-4,6-O-benzylidene-3-deoxy-2-O-methyl-β-D-galactopyranosyl)-3-carboxy-1H-1,2-pyrazol-1-yl]-5-chloro-2-(trifluoromethyl)benzene

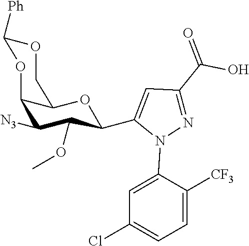

To a solution 1-[5-(3-azido-4,6-O-benzylidene-3-deoxy-2-O-methyl-β-D-galactopyranosyl)-3-ethoxycarbonyl-1H-1,2-pyrazol-1-yl]-5-chloro-2-(trifluoromethyl)benzene (640 mg, 1.05 mmol) in MeOH/THF/H₂O (30 mL, 1:1:1) NaOH (126 mg, 3.16 mmol) was added and the mixture was stirred 2 h at rt. The mixture was concentrated, and water (20 mL) was added. The mixture was acidified (pH=6) by addition of HCl (2M) and extracted with EtOAc (2×40 mL). The combined organic phases were dried over Na₂SO₄, concentrated, and purified by column chromatography (MeOH/DCM=0/1˜1/8, Silica-CS 25 g, 25 m/min, silica gel, UV 254) to afford the product (550 mg, 90%). ESI-MS m/z calcd for [C₂₅H₂₁ClF₃N₅O₆][M+H]⁺: 580.1; found: 580.2. ¹H NMR (400 MHz, Methanol-d₄) δ7.81 (d, J=8.4 Hz, 1H), 7.72-7.48 (m, 4H), 7.39-7.35 (m, 3H), 7.09 (s, 1H), 5.59 (s, 1H), 4.32 (d, J=2.8 Hz, 1H), 4.21-4.17 (m, 1H), 4.05-3.69 (m, 3H), 3.52-3.30 (m, 5H).

### 1-{5-(3-Azido-4,6-O-benzylidene-3-deoxy-2-O-methyl-β-D-galactopyranosyl)-3-{{[2-(trimethylsilyl)ethoxy]carbonyl}amino}-1H-1,2-pyrazol-1-yl}-5-chloro-2-(trifluoromethyl)benzene

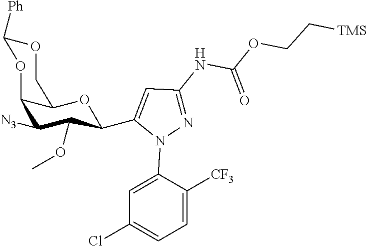

To a solution of 1-[5-(3-azido-4,6-O-benzylidene-3-deoxy-2-O-methyl-β-D-galactopyranosyl)-3-carboxy-1H-1,2-pyrazol-1-yl]-5-chloro-2-(trifluoromethyl)benzene (250 mg, 0.43 mmol) in 1,4-dioxane (6.0 mL) diphenylphosphoryl azide (0.186 mL, 0.86 mmol) and Et₃N (0.120 mL, 0.86 mmol) were added and the mixture was stirred 2 h at 50° C. 2-Trimethylsilylethanol (255 mg, 2.16 mmol) was added and the mixture was stirred 2 h at 100° C. The mixture was concentrated and water (20 mL) was added. The mixture was extracted with EtOAc (2×20 mL). The combined organic phases were dried over Na₂SO₄, concentrated, and purified by column chromatography (EtOAc/PE=0/1˜1/2, Silica-CS 25 g, 25 mL/min, silica gel, UV 254) to afford the product (162 mg, 54%). ESI-MS m/z calcd for [C₃₀H₃₄ClF₃N₆O₆Si] [M+H]⁺: 695.2; found: 695.3. ¹H NMR (400 MHz, Chloroform-d) δ 7.65-7.35 (m, 8H), 7.03-6.91 (m, 2H), 5.46 (s, 1H), 4.16-4.13 (m, 4H), 3.92-3.72 (m, 3H), 3.37-3.23 (m, 5H), 1.00 (t, J=8.8 Hz, 2H), 0.01 (s, 9H).

### 1-[3-Amino-5-(3-azido-4,6-O-benzylidene-3-deoxy-2-O-methyl-β-D-galactopyranosyl)-1H-1,2-pyrazol-1-yl]-5-chloro-2-(trifluoromethyl)benzene

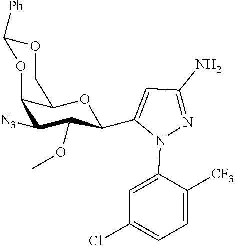

To a solution of 1-{5-(3-azido-4,6-O-benzylidene-3-deoxy-2-O-methyl-β-D-galactopyranosyl)-3-{{[2-(trimethylsilyl)ethoxy]carbonyl}amino}-1H-1,2-pyrazol-1-yl}-5-chloro-2-(trifluoromethyl)benzene (850 mg, 1.22 mmol) in THF (20.0 mL) TBAF (2.5 mL, 1M in THF) was added and the mixture was stirred 2 h at 50° C. Water (30 mL) was added and the mixture was extracted with EtOAc (3×30 mL). The combined organic phases were dried, concentrated, and purified by column chromatography (PE/EtOAc=10/1˜1/2, Silica-CS 20 g, 30 m/min, silica gel, UV254) to afford the product (495 mg, 74%). ESI-MS m/z calcd for [C₂₄H₂₂ClF₃N₆O₄] [M+H]⁺: 551.1; found: 551.2. ¹H NMR (400 MHz, Chloroform-d) δ 7.67 (d, J=8.8 Hz, 1H), 7.52-7.37 (m, 7H), 6.06 (s, 1H), 5.54 (s, 1H), 4.25-4.21 (m, 2H), 3.99 (d, J=12.4 Hz, 1H), 3.91 (d, J=9.6 Hz, 1H), 3.76 (br s, 1H), 3.39 (s, 3H), 3.34-3.30 (m, 2H).

### 1-[5-(3-Azido-4,6-O-benzylidene-3-deoxy-2-O-methyl-β-D-galactopyranosyl)-3-chloro-1H-1,2-pyrazol-1-yl]-5-chloro-2-(trifluoromethyl)benzene

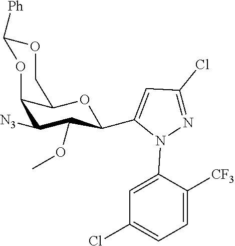

To a solution of 1-[3-amino-5-(3-azido-4,6-O-benzylidene-3-deoxy-2-O-methyl-β-D-galactopyranosyl)-1H-1,2-pyrazol-1-yl]-5-chloro-2-(trifluoromethyl)benzene (100 mg, 0.18 mmol) in MeCN (5.0 mL) copper(I) chloride (53.9 mg, 0.55 mmol) and lithium chloride (1.54 mg, 0.036 mmol) were added. The mixture was cooled to 0° C. and pentyl nitrite (31.9 mg, 0.27 mmol) was added. The mixture was stirred 1 h at 0° C. and 1 h at rt. Water (20 mL) was added and the mixture was extracted with EtOAc (3×30 mL). The combined organic phases were dried, concentrated, and purified by column chromatography (PE/EtOAc=10/1˜3/1, Silica-CS 20 g, 30 mL/min, silica gel, UV 254) to afford the product (84 mg, 81%). ESI-MS m/z calcd for [C₂₄H₂₀Cl₂F₃N₅O₄] [M+H]⁺: 570.1; found: 570.1. ¹H NMR (400 MHz, Chloroform-d) δ 7.68 (d, J=6.8 Hz, 1H), 7.51-7.37 (m, 7H), 6.56 (br s, 1H), 5.54 (s, 1H), 4.24-4.18 (m, 2H), 4.00-3.97 (m, 2H), 3.86-3.63 (m, 1H), 3.42-3.30 (m, 5H).

### 1-{5-{4,6-O-Benzylidene-3-[4-(4-chlorothiazol-2-yl)-1H-1,2,3-triazol-1-yl]-3-deoxy-2-O-methyl-β-D-galactopyranosyl}-3-chloro-1H-1,2-pyrazol-1-yl}-5-chloro-2-(trifluoromethyl)benzene

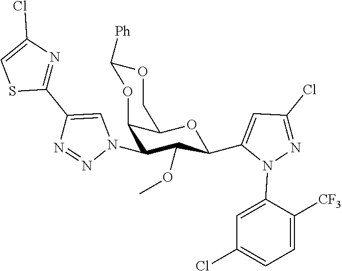

To a solution of 1-[5-(3-azido-4,6-O-benzylidene-3-deoxy-2-O-methyl-β-D-galactopyranosyl)-3-chloro-1H-1,2-pyrazol-1-yl]-5-chloro-2-(trifluoromethyl)benzene (84 mg, 0.15 mmol) in DMF (5 mL) 2-(4-chlorothiazol-2-yl)ethynyltrimethylsilane (63.6 mg, 0.30 mmol), copper(II) sulfate pentahydrate (36.8 mg, 0.15 mmol) and (+)-sodium L-ascorbate (29.2 mg, 0.15 mmol) were added and the mixture was stirred overnight at rt. The mixture was concentrated and purified by column chromatography (PE/EtOAc=10/1˜1/1, Silica-CS 4 g, 10 mL/min, silica gel, UV 254) to afford the product (69 mg, 66%). ESI-MS m/z calcd for [C₂₉H₂₂Cl₃F₃N₄O₅S] [M+H]⁺: 713.0; found: 713.2 ¹H NMR (400 MHz, Chloroform-d) δ 8.41 (s, 1H), 7.76-7.71 (m, 1H), 7.59-7.42 (m, 7H), 7.11 (s, 1H), 6.60 (br s, 1H), 5.47 (s, 1H), 4.95 (d, J=8.0 Hz, 1H), 4.38 (d, J=3.2 Hz, 1H), 4.28 (d, J=12.4 Hz, 1H), 4.20-4.17 (m, 1H), 4.09-4.01 (m, 2H), 3.55 (s, 1H), 2.89 (s, 3H).

### Intermediate 26

### 1-[5-(3-Azido-4,6-O-benzylidene-3-deoxy-2-O-methyl-β-D-galactopyranosyl)-3-bromo-1H-1,2-pyrazol-1-yl]-5-chloro-2-(trifluoromethyl)benzene

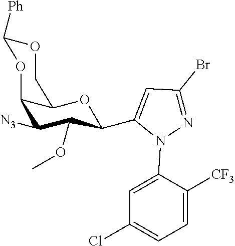

To a solution of 1-[3-amino-5-(3-azido-4,6-O-benzylidene-3-deoxy-2-O-methyl-3-D-galactopyranosyl)-1H-1,2-pyrazol-1-yl]-5-chloro-2-(trifluoromethyl)benzene (380 mg, 0.69 mmol) in MeCN (10.0 mL) copper(I) bromide (198 mg, 1.38 mmol) and copper(II) bromide (15.4 mg, 0.069 mmol) were added. The mixture was cooled to 0° C. and pentyl nitrite (121 mg, 1.03 mmol) was added. The mixture was stirred 1 h at 0° C. and 1 h at rt. Water (20 mL) was added and the mixture was extracted with EtOAc (3×30 mL). The combined organic phases were dried, concentrated, and purified by column chromatography (PE/EtOAc=10/1˜3/1, Silica-CS 20 g, 30 mL/min, silica gel, UV 254) to afford the product (261 mg, 62%). ESI-MS m/z calcd for [C₂₄H₂₀BrClF₃N₅O₄] [M+H]⁺: 614.0; found: 614.3. ¹H NMR (400 MHz, Chloroform-d) δ 7.67 (d, J=7.2 Hz, 1H), 7.52-7.37 (m, 7H), 6.65 (br s, 1H), 5.53 (s, 1H), 4.25-4.21 (m, 2H), 4.02-3.96 (m, 2H), 3.88-3.63 (m, 1H), 3.42-3.30 (m, 5H).

### 1-{5-{4,6-O-Benzylidene-3-[4-(4-chlorothiazol-2-yl)-1H-1,2,3-triazol-1-yl]-3-deoxy-2-O-methyl-β-D-galactopyranosyl}-3-bromo-1H-1,2-pyrazol-1-yl}-5-chloro-2-(trifluoromethyl)benzene

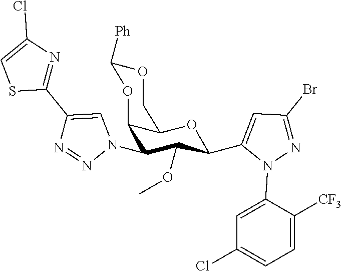

To a solution of 1-[5-(3-azido-4,6-O-benzylidene-3-deoxy-2-O-methyl-β-D-galactopyranosyl)-3-bromo-1H-1,2-pyrazol-1-yl]-5-chloro-2-(trifluoromethyl)benzene (76 mg, 0.12 mmol) in DMF (5 mL) 2-(4-chlorothiazol-2-yl)ethynyltrimethylsilane (53.3 mg, 0.25 mmol), copper(II) sulfate pentahydrate (30.9 mg, 0.12 mmol) and (+)-sodium L-ascorbate (24.5 mg, 0.12 mmol) were added and the mixture was stirred overnight at rt. The mixture was concentrated and purified by column chromatography (PE/EtOAc=10/1˜1/1, Silica-CS 4 g, 10 mL/min, silica gel, UV 254) to afford the product (72 mg, 77%). ESI-MS m/z calcd for [C₂₉H₂₂BrCl₂F₃N₆O₄S] [M+H]⁺: 759.0; found: 759.1 ¹H NMR (400 MHz, Chloroform-d) δ 8.41 (s, 1H), 7.76-7.71 (m, 1H), 7.59-7.40 (m, 7H), 7.11 (s, 1H), 6.69 (br s, 1H), 5.47 (s, 1H), 4.95 (d, J=8.0 Hz, 1H), 4.38 (d, J=2.8 Hz, 1H), 4.27 (d, J=12.4 Hz, 1H), 4.21-4.19 (m, 1H), 4.05-4.01 (m, 2H), 3.56 (br s, 1H), 2.88 (s, 3H).

### Intermediate 27

### 1-{5-{4,6-O-Benzylidene-3-[4-(4-chlorothiazol-2-yl)-1H-1,2,3-triazol-1-yl]-3-deoxy-2-O-methyl-β-D-galactopyranosyl}-3-(1-methylethenyl)-1H-1,2-pyrazol-1-yl}-5-chloro-2-(trifluoromethyl)benzene

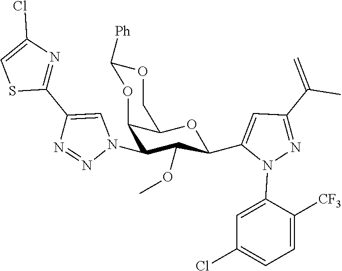

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-bromo-1H-1,2-pyrazol-1-yl}-5-chloro-2-(trifluoromethyl)benzene (85 mg, 0.11 mmol) in 1,4-dioxane/H₂O (6.0 mL, 5:1) 2-isopropenyl-4,4,5,5-tetramethyl-1,3,2-dioxaborolane (94.2 mg, 0.56 mmol), Pd(dppf)Cl₂ (8.2 mg, 0.011 mmol) and K₂CO₃ (46.5 mg, 0.34 mmol) were added. The mixture was purged three times with nitrogen, and stirred 16 h at 70° C. The mixture was concentrated and purified by column chromatography (EtOAc/PE=0/1˜1/1, Silica-CS 12 g, 20 m/min, silica gel, UV 254) to afford the product (55 mg, 68%). ESI-MS m/z calcd for [C₃₂H₂₇Cl₂F₃N₆O₄S] [M+H]⁺: 719.1; found: 719.2.

### Intermediate 28

### 1-{5-{4,6-O-Benzylidene-3-[4-(4-chlorothiazol-2-yl)-1H-1,2,3-triazol-1-yl]-3-deoxy-2-O-methyl-β-D-galactopyranosyl}-3-isopropyl-1H-1,2-pyrazol-1-yl}-5-chloro-2-(trifluoromethyl)benzene

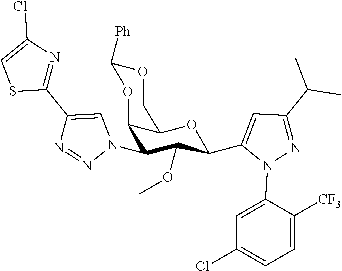

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-(1-methylethenyl)-1H-1,2-pyrazol-1-yl}-5-chloro-2-(trifluoromethyl)benzene (55.0 mg, 0.076 mmol) in MeOH (10 mL) platinum(IV) oxide (5.2 mg, 0.023 mmol) was added and the mixture was stirred 72 h at rt under hydrogen atmosphere. The mixture was filtered through a celite pad and concentrated. The residue was purified by column chromatography (EtOAc/PE=0/1˜1/2, Silica-CS 20 g, 20 mL/min, silica gel, UV 254) to afford the product (30 mg, 55%). ESI-MS m/z calcd for [C₃₂H₂₉Cl₂F₃N₆O₄S] [M+H]⁺: 721.2; found: 721.2.

### Intermediate 29

### 1-{5-{4,6-O-Benzylidene-3-[4-(4-chlorothiazol-2-yl)-1H-1,2,3-triazol-1-yl]-3-deoxy-2-O-methyl-β-D-galactopyranosyl}-3-ethenyl-1H-1,2-pyrazol-1-yl}-5-chloro-2-(trifluoromethyl)benzene

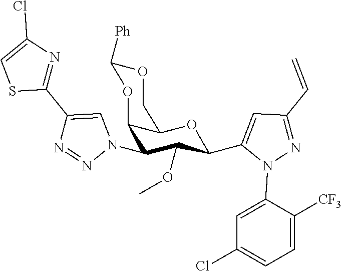

To a solution of 1-{5-{4,6-O-benzylidene-3-[4-(4-chlorothiazol-2-yl)-1H-1,2,3-triazol-1-yl]-3-deoxy-2-O-methyl-β-D-galactopyranosyl}-3-bromo-1H-1,2-pyrazol-1-yl}-5-chloro-2-(trifluoromethyl)benzene (100 mg, 0.13 mmol) in toluene/H₂O (6.0 mL, 5:1) 4,4,5,5-tetramethyl-2-vinyl-1,3,2-dioxaborolane (102 mg, 0.66 mmol), Pd(dppf)Cl₂ (9.65 mg, 0.013 mmol) and K₂CO₃ (54.7 mg, 0.40 mmol) were added. The mixture was purged three times with nitrogen and stirred 1 h at 100° C. in a microwave reactor. The mixture was concentrated and purified by column chromatography (EtOAc/PE=0/1˜1/1, Silica-CS 12 g, 20 mL/min, silica gel, UV 254). The obtained product was further purified by prep HPLC (MeCN/H₂O (10 mmol/L NH4HCO3), X-Select 10 μM 19*250 mm, 20 mL/min, UV 254) to afford the product (33 mg, 36%). ESI-MS m/z calcd for [C₃₁H₂₅Cl₂F₃N₆O₄S] [M+H]⁺: 705.1; found: 705.2. ¹H NMR (400 MHz, Chloroform-d) δ 8.42 (s, 1H), 7.74 (d, J=8.4 Hz, 1H), 7.58-7.43 (m, 7H), 7.11 (s, 1H), 6.78-6.70 (m, 2H), 5.85 (d, J=17.6 Hz, 1H), 5.49 (s, 1H), 5.42 (dd, J=10.8, 1.2 Hz, 1H), 4.96 (d, J=7.6 Hz, 1H), 4.39 (d, J=2.8 Hz, 1H), 4.32 (d, J=12.4 Hz, 1H), 4.20-4.02 (m, 3H), 3.56 (s, 1H), 2.89 (s, 3H).

### Intermediate 31

### 1-[3-Aminocarbonyl-5-(3-azido-4,6-O-benzylidene-3-deoxy-2-O-methyl-β-D-galactopyranosyl)-1H-1,2-pyrazol-1-yl]-5-chloro-2-(trifluoromethyl)benzene

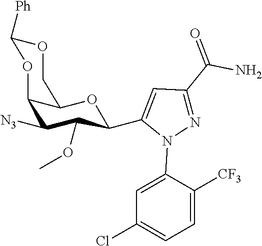

To a solution 1-[5-(3-azido-4,6-O-benzylidene-3-deoxy-2-O-methyl-β-D-galactopyranosyl)-3-ethoxycarbonyl-1H-1,2-pyrazol-1-yl]-5-chloro-2-(trifluoromethyl)benzene (150 mg, 0.25 mmol) in MeOH (2 mL) a solution of NH₃ in MeOH (3 mL, 7 M) was added and the mixture was stirred 24 h at 30° C. The mixture was concentrated and purified by column chromatography (EtOAc/PE=0/1˜1/1, Silica-CS 12 g, 20 m/min, silica gel, UV 254) to afford the product (92 mg, 64%). ESI-MS m/z calcd for [C₂₅H₂₂ClF₃N₆O₅] [M+H]⁺: 579.1; found: 579.1. ¹H NMR (400 MHz, Chloroform-d) δ 7.72-7.69 (m, 1H), 7.68-7.50 (m, 3H), 7.48-7.38 (m, 5H), 7.17 (s, 1H), 6.73 (br s, 1H), 5.52 (s, 1H), 4.22 (d, J=3.2 Hz, 1H), 4.19 (d, J=12.8 Hz, 1H), 4.06 (d, J=9.6 Hz, 1H), 3.99 (d, J=12.0 Hz, 1H), 3.82 (br s, 1H), 3.39-3.28 (m, 5H).

### 1-[5-(3-Azido-4,6-O-benzylidene-3-deoxy-2-O-methyl-β-D-galactopyranosyl)-3-cyano-1H-1,2-pyrazol-1-yl]-5-chloro-2-(trifluoromethyl)benzene

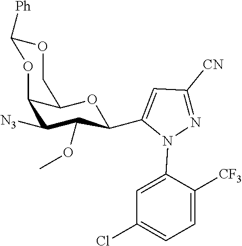

To a solution 1-[3-aminocarbonyl-5-(3-azido-4,6-O-benzylidene-3-deoxy-2-O-methyl-β-D-galactopyranosyl)-1H-1,2-pyrazol-1-yl]-5-chloro-2-(trifluoromethyl)benzene (92 mg, 0.16 mmol) in THF (10 mL) pyridine (62.9 mg, 0.80 mmol) and trifluoroacetic anhydride (0.221 mL, 1.59 mmol) were added and the mixture was stirred 2 h at rt. The mixture was concentrated, and the obtained residue was dissolved in EtOAc (20 mL). The mixture was washed with water and brine, dried over anhydrous Na₂SO₄, concentrated, and purified by column chromatography (EtOAc/PE=0/1˜1/2, Silica-CS 4 g, 10 mL/min, silica gel, UV 254) to afford the product (66 mg, 74%). ESI-MS m/z calcd for [C₂₅H₂₀ClF₃N₆O₄] [M+H]⁺: 561.1; found: 561.2. ¹H NMR (400 MHz, Chloroform-d) δ 7.75-7.64 (m, 1H), 7.56-7.52 (m 1H), 7.48-7.39 (m, 6H), 7.04-6.96 (m, 1H), 5.54 (s, 1H), 4.25 (d, J=2.8 Hz, 1H), 4.18-4.14 (m, 1H), 4.06 (d, J=9.6 Hz, 1H), 3.99 (d, J=12.0 Hz, 1H), 3.85-3.68 (m, 1H), 3.42-3.30 (m, 5H).

### 1-{5-{4,6-O-Benzylidene-3-[4-(4-chlorothiazol-2-yl)-1H-1,2,3-triazol-1-yl]-3-deoxy-2-O-methyl-β-D-galactopyranosyl}-3-cyano-1H-1,2-pyrazol-1-yl}-5-chloro-2-(trifluoromethyl)benzene

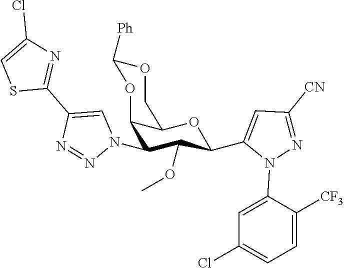

To a solution of 1-[5-(3-azido-4,6-O-benzylidene-3-deoxy-2-O-methyl-β-D-galactopyranosyl)-3-cyano-1H-1,2-pyrazol-1-yl]-5-chloro-2-(trifluoromethyl)benzene (33 mg, 0.059 mmol) in DMF (2 mL) 2-(4-chlorothiazol-2-yl)ethynyltrimethylsilane (16.5 mg, 0.077 mmol), copper(II) sulfate pentahydrate (14.7 mg, 0.059 mmol) and (+)-sodium L-ascorbate (11.7 mg, 0.059 mmol) were added and the mixture was stirred overnight at rt. The mixture was concentrated and purified by column chromatography (PE/EtOAc=1/0˜1/1, Silica-CS 4 g, 10 m/min, silica gel, UV 254) to afford the product (32 mg, 77%). ESI-MS m/z calcd for [C₃₀H₂₂Cl₂F₃N₇O₄S] [M+H]⁺: 704.1; found: 704.0 ¹H NMR (400 MHz, Chloroform-d) δ 8.42 (s, 1H), 7.79-7.59 (m, 3H), 7.48-7.26 (m, 5H), 7.12 (s, 1H), 7.04-6.97 (m, 1H), 5.48 (s, 1H), 4.99-4.94 (m, 1H), 4.38 (d, J=2.8 Hz, 1H), 4.27-4.24 (m, 2H), 4.05-3.99 (m, 2H), 3.56 (br s, 1H), 2.83 (s, 3H).

### Intermediate 32

### 5-Chloro-2-(trifluoromethyl)-3-nitropyridine

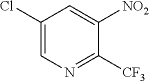 [SMILES: O=[N+]([O-])c1cc(Cl)cnc1C(F)(F)F]

A mixture of 2-bromo-5-chloro-3-nitropyridine (9.80 g, 41.3 mmol), methyl 2,2-difluoro-2-fluorosulfonylacetate (11.9 g, 61.9 mmol) and CuI (9.43 g, 49.5 mmol) in DMF (100 mL) was stirred 2 h at 100° C. After cooling to rt, the mixture was poured into water (300 mL) and then extracted with EtOAc (3×100 mL). The combined organic phases were washed with water (100 mL) and brine (100 mL), dried over Na₂SO₄, concentrated, and purified by column chromatography (EtOAc/PE=0/1˜1/4, Silica-CS 80 g, 40 mL/min, silica gel, UV 254) to give the product (8.8 g, 94%). ESI-MS m/z calcd for [C₆H₂ClF₃N₂O₂] [M]: 226.0; found: 226.0. ¹H NMR (400 MHz, Chloroform-d) δ 8.79 (d, J=2.0 Hz, 1H), 8.16 (d, J=1.6 Hz, 1H).

### 3-Amino-5-chloro-2-(trifluoromethyl)pyridine

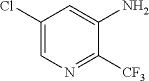 [SMILES: Nc1cc(Cl)cnc1C(F)(F)F]

A solution of 5-chloro-2-(trifluoromethyl)-3-nitropyridine (8.8 g, 38.8 mmol), ammonium hydrochloride (12.5 g, 233 mmol) and iron (10.8 g, 194 mmol) in EtOH (100 mL) and H₂O (10 mL) was stirred 6 h at 85° C. The mixture was filtered, concentrated, and purified by column chromatography (EtOAc/PE=0/1˜1/4, Silica-CS 80 g, 40 mL/min, silica gel, UV 254) to give the product (7.1 g, 93%). ESI-MS m/z calcd for [C₆H₄ClF₃N₂] [M+H]⁺: 197.0; found: 197.2. ¹H NMR (400 MHz, Chloroform-d) δ 7.92 (d, J=1.6 Hz, 1H), 7.04 (d, J=2.0 Hz, 1H), 4.25 (br s, 2H).

### [5-Chloro-2-(trifluoromethyl)-3-pyridyl]hydrazine

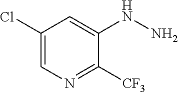 [SMILES: NNc1cc(Cl)cnc1C(F)(F)F]

To a cooled (−5° C.) solution of 3-amino-5-chloro-2-(trifluoromethyl)pyridine (2.50 g, 12.7 mmol) in acetic acid (8.0 mL) concentrated HCl (16.0 mL) was added. A solution of NaNO₂ (1.14 g, 16.5 mmol) in water (4.0 mL) was added dropwise over 5 min. The mixture was stirred 45 min at −5° C. Then a cooled (0° C.) solution of tin(II) chloride dihydrate (2.87 g, 12.7 mmol) in concentrated HCl (8.0 mL) was added dropwise over 10 min. The mixture was stirred 2 h at −5° C. Then aqueous NaOH (5 M) was added dropwise at −5° C. to adjust pH to 8. The mixture was extracted with EtOAc (2×100 mL). The combined organic phases were washed with water (100 mL) and brine (100 mL), dried over Na₂SO₄, evaporated, and purified by column chromatography (PE/EtOAc=1/0˜4/1, silica-CS 20 g 20 mL/min, silica gel, UV 254 nm) to afford the product (1.8 g, 67%). ESI-MS m/z calcd for [C₆H5ClF₃N3] [M+H]⁺: 212.0; found: 212.2. ¹H NMR (400 MHz, Chloroform-d) δ 7.88 (d, J=2.0 Hz, 1H), 7.81 (d, J=2.0 Hz, 1H), 5.94 (s, 1H), 3.65 (s, 2H).

### Intermediate 33

### 5-Bromo-3-nitro-2-(trifluoromethyl)pyridine

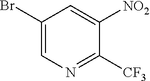 [SMILES: O=[N+]([O-])c1cc(Br)cnc1C(F)(F)F]

A solution of 2,5-dibromo-3-nitropyridine (2.00 g, 7.09 mmol), methyl 2,2-difluoro-2-(fluorosulfonyl)acetate (2.05 g, 10.6 mmol) and CuI (1.62 g, 8.51 mmol) in DMF (20.0 mL) was stirred 2 h at 100° C. After cooling to rt, the mixture was poured into water (50 mL) and extracted with EtOAc (3×50 mL). The combined organic phases were washed with water (50 mL) and brine (50 mL), dried over Na₂SO₄, evaporated and purified by column chromatography (EtOAc/PE=0/1˜1/4, Silica-CS 20 g, 20 mL/min, silica gel, UV 254) to give the product (1.8 g, 94%). ESI-MS m/z calcd for [C₆H₂BrF₃N₂O₂] [M]: 269.9; found: 270.0. ¹H NMR (400 MHz, Chloroform-d) δ 8.90 (d, J=1.6 Hz, 1H), 8.30 (d, J=1.6 Hz, 1H).

### 5-Bromo-2-(trifluoromethyl)pyridin-3-amine

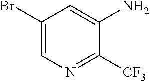 [SMILES: Nc1cc(Br)cnc1C(F)(F)F]

To a solution of 5-bromo-3-nitro-2-(trifluoromethyl)pyridine (1.8 g, 6.64 mmol) in EtOH/H₂O (22 mL, 10:1) iron (1.86 g, 33.2 mmol) and NH₄Cl (2.13 g, 39.9 mmol) were added and the mixture was stirred 6 h at 85° C. The mixture was concentrated and purified by column chromatography (PE/EtOAc=1/0˜4/1, Silica-CS 20 g, 20 mL/min, silica gel, UV 254) to give the product (1.45 g, 91%). ESI-MS m/z calcd for [C₆H₄BrF₃N₂] [M+H]⁺: 241.0; found: 240.9. ¹H NMR (400 MHz, Chloroform-d) δ 8.01 (d, J=1.6 Hz, 1H), 7.21 (d, J=1.6 Hz, 1H), 4.22 (br s, 2H).

### [5-Bromo-2(trifluoromethyl)pyrid-3-yl]hydrazine

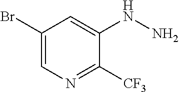 [SMILES: NNc1cc(Br)cnc1C(F)(F)F]

To a cooled (−5° C.) solution of 5-bromo-2-(trifluoromethyl)pyridin-3-amine (600 mg, 2.49 mmol) in acetic acid (3.0 mL) concentrated HCl (9.0 mL) was added followed by dropwise addition of a solution of NaNO₂ (223 mg, 3.24 mmol) in water (1 mL). The resulting mixture was stirred 45 min at −5° C. Tin(II) chloride dihydrate (1.69 g, 7.47 mmol) dissolved in concentrated HCl (3.0 mL) was added dropwise at 0° C. The resulting mixture was stirred 2 h at −5° C. Water (30 mL) was added and the pH was adjusted to 8 by dropwise addition of aq NaOH (5 M). The aqueous phase was extracted with EtOAc (2×50 mL). The combined organic phases were washed with water (50 mL) and brine (50 mL), dried over Na₂SO₄, evaporated, and purified by column chromatography (EtOAc/PE=0/1˜1/4, Silica-CS 20 g, 20 m/min, silica gel, UV 254) to afford the product (350 mg, 55%). ESI-MS m/z calcd for [C₆H₅BrF₃N₃] [M+H]⁺: 256.0; found: 256.1. ¹H NMR (400 MHz, Chloroform-d) δ 7.98 (s, 2H), 5.93 (s, 1H), 3.64 (br s, 2H).

### Intermediate 36

### 1-[5-(3-Azido-3-deoxy-2-O-methyl-β-D-galactopyranosyl)-1H-1,2-pyrazol-1-yl]-4,5-dichloro-2-(trifluoromethyl)benzene

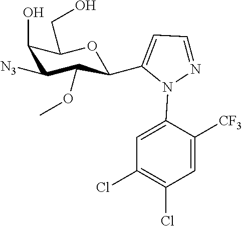

A solution of 3,7-anhydro-5-azido-6,8-O-benzylidene-5-deoxy-4-O-methyl-D-glycero-L-manno-2-octulose (85 mg, 0.26 mmol) in N,N-dimethylformamide dimethyl acetal (3 mL) was stirred 3 h at 80° C. The mixture was concentrated to dryness and the residue was dissolved in EtOH (5.0 mL). [4,5-Dichloro-2-(trifluoromethyl)phenyl]hydrazine (75 mg, 0.31 mmol) and concentrated HCl (0.2 mL) were added and the mixture was stirred 2 h at 80° C. The mixture was cooled to rt, poured into aq NaHCO₃ (20 mL) and extracted with EtOAc (2×30 mL). The combined organic phases were washed with brine, dried with Na₂SO₄, and concentrated. The residue was purified by column chromatography (PE/EtOAc=1/0˜2/1, Silica-CS 4 g, 12 mL/min, silica gel, UV 254) to afford the product (20 mg, 16%). ESI-MS m/z calcd for [C₁₇H₁₆Cl₂F₃N₅O₄] [M+H]⁺: 482.1; found: 482.2.

### Intermediate 37

### 1-[5-(3-Azido-3-deoxy-2-O-methyl-β-D-galactopyranosyl)-1H-1,2-pyrazol-1-yl]-5-bromo-4-fluoro-2-(trifluoromethyl)benzene

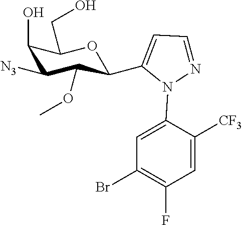

A solution of 3,7-anhydro-5-azido-6,8-O-benzylidene-5-deoxy-4-O-methyl-D-glycero-L-manno-2-octulose (80 mg, 0.24 mmol) in N,N-dimethylformamide dimethyl acetal (3 mL) was stirred 3 h at 80° C. The mixture was concentrated to dryness and the residue was dissolved in EtOH (5.0 mL). [5-Bromo-4-fluoro-2-(trifluoromethyl)phenyl]hydrazine (72.1 mg, 0.26 mmol) and concentrated HCl (0.2 mL) were added and the mixture was stirred 2 h at 80° C. The mixture was cooled to rt, poured into aq NaHCO₃ (20 mL) and extracted with EtOAc (2×30 mL). The combined organic phases were washed with brine, dried with Na₂SO₄, and concentrated. The residue was purified by column chromatography (PE/EtOAc=1/0˜1/2, Silica-CS 4 g, 12 m/min, silica gel, UV 254) to afford the product (21 mg, 17%). ESI-MS m/z calcd for [C₁₇H₁₆BrF₄N₅O₄] [M+H]⁺: 510.0; found: 510.1.

### Intermediate 39

### 1-{3-Aminocarbonyl-5-{4,6-O-benzylidene-3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene

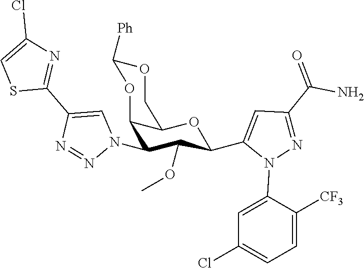

To a solution of 1-[3-aminocarbonyl-3-(3-azido-4,6-O-benzylidene-3-deoxy-2-O-methyl-3-D-galactopyranosyl)-1H-1,2-pyrazol-1-yl]-5-chloro-2-(trifluoromethyl)benzene (41 mg, 0.071 mmol) in DMF (2 mL) 2-(4-chlorothiazol-2-yl)ethynyltrimethylsilane (19.9 mg, 0.092 mmol), copper(II) sulfate pentahydrate (17.7 mg, 0.071 mmol) and (+)-sodium L-ascorbate (14.0 mg, 0.071 mmol) were added and the mixture was stirred overnight at rt. The mixture was concentrated and purified by column chromatography (PE/EtOAc=1/0˜1/1, Silica-CS 4 g, 10 mL/min, silica gel, UV 254) to afford the product (29 mg, 57%). ESI-MS m/z calcd for [C₃₀H₂₄Cl₂F₃N₇O₅S][M+H]⁺: 722.1; found: 722.1. ¹H NMR (400 MHz, Chloroform-d) δ 8.70 (s, 1H), 7.79 (d, J=8.4 Hz, 1H), 7.68-7.57 (m, 2H), 7.50-7.36 (m, 5H), 7.34 (s, 1H), 7.09 (s, 1H), 6.76 (br s, 1H), 6.01 (br s, 1H), 5.44 (s, 1H), 4.98 (dd, J=10.4, 3.2 Hz, 1H), 4.44-4.36 (m, 2H), 4.28-4.21 (m, 2H), 4.02 (d, J=12.4 Hz, 1H), 3.55 (s, 1H), 2.93 (s, 3H).

### REFERENCES

Bennett, D.; Bargagli, E.; Bianchi, N.; Landi, C.; Fossi, A.; Fui, A.; Sestini, P.; Refini, R. M.; Rottoli, P. Elevated Level of Galectin-1 in Bronchoalveolar Lavage of Patients with Idiopathic Pulmonary Fibrosis. Respir Physiol Neurobiol 2019, 273, 103323. https://doi.org/10.1016/j.resp.2019.103323.
Blanchard, H.; Yu, X.; Collins, P. M.; Bum-Erdene, K. Galectin-3 Inhibitors: A Patent Review (2008-Present). Expert Opin. Ther. Patents 2014, 24 (10), 1053-1065. https://doi.org/10.1517/13543776.2014.947961.
Blanchard, H.; Bum-Erdene, K.; Bohari, M. H.; Yu, X. Galectin-1 Inhibitors and Their Potential Therapeutic Applications: A Patent Review. Expert Opin. Ther. Patents 2016, 26 (5), 537-554. https://doi.org/10.1517/13543776.2016.1163338.
Daley, D.; Mani, V. R.; Mohan, N.; Akkad, N.; Ochi, A.; Heindel, D. W.; Lee, K. B.; Zambirinis, C. P.; Pandian, G. S. D. B.; Savadkar, S.; Torres-Hernandez, A.; Nayak, S.; Wang, D.; Hundeyin, M.; Diskin, B.; Aykut, B.; Werba, G.; Barilla, R. M.; Rodriguez, R.; Chang, S.; Gardner, L.; Mahal, L. K.; Ueberheide, B.; Miller, G. Dectin-1 Activation on Macrophages by Galectin-9 Promotes Pancreatic Carcinoma and Peritumoral Immune-Tolerance. Nat Med 2017, 23 (5), 556-567. https://doi.org/10.1038/nm.4314.
Dings, R. P. M., Miller, M. C., Griffin, R. J. & Mayo, K. H. Galectins as Molecular Targets for Therapeutic Intervention. IJMS 19, (2018).
Dubd-Delarosbil, C.; St-Pierre, Y. The Emerging Role of Galectins in High-Fatality Cancers. Cell. Mol. Life Sci. 2017, 75 (7), 1215-1226. https://doi.org/10.1007/s00018-017-2708-5.
Drake, I.; Fryk, E.; Strindberg, L.; Lundqvist, A.; Rosengren, A. H.; Groop, L.; Ahlqvist, E.; Bordn, J.; Orho-Melander, M.; Jansson, P.-A. The Role of Circulating Galectin-1 in Type 2 Diabetes and Chronic Kidney Disease: Evidence from Cross-Sectional, Longitudinal and Mendelian Randomisation Analyses. Diabetologia 2022, 65 (1), 128-139. https://doi.org/10.1007/s00125-021-05594-1.
Hsu, Y.-A.; Chang, C.-Y.; Lan, J.-L.; Li, J.-P.; Lin, H.-J.; Chen, C.-S.; Wan, L.; Liu, F.-T. Amelioration of Bleomycin-Induced Pulmonary Fibrosis via TGF-β-Induced Smad and Non-Smad Signaling Pathways in Galectin-9-Deficient Mice and Fibroblast Cells. J Biomed Sci 2020, 27 (1), 24. https://doi.org/10.1186/s12929-020-0616-8.
Johannes, L.; Jacob, R.; Leffler, H. Galectins at a Glance. J Cell Sci 2018, 131 (9), jcs208884. https://doi.org/10.1242/jcs.208884.
Kathiriya, J. J.; Nakra, N.; Nixon, J.; Patel, P. S.; Vaghasiya, V.; Alhassani, A.; Tian, Z.; Allen-Gipson, D.; Dav, V. Galectin-1 Inhibition Attenuates Profibrotic Signaling in Hypoxia-Induced Pulmonary Fibrosis. Cell Death Discovery 2017, 3, 17010-17013. https://doi.org/10.1038/cddiscovery.2017.10.
Li, P.; Liu, S.; Lu, M.; Bandyopadhyay, G.; Oh, D.; Imamura, T.; Johnson, A. M. F.; Sears, D.; Shen, Z.; Cui, B.; Kong, L.; Hou, S.; Liang, X.; Iovino, S.; Watkins, S. M.; Ying, W.; Osborn, O.; Wollam, J.; Brenner, M.; Olefsky, J. M. Hematopoietic-Derived Galectin-3 Causes Cellular and Systemic Insulin Resistance. Cell 2016, 167 (4), 973-984. e12. https://doi.org/10.1016/j.cell.2016.10.025.
Sethi, A.; Sanam, S.; Alvala, R.; Alvala, M. An Updated Patent Review of Galectin-1 and Galectin-3 Inhibitors and Their Potential Therapeutic Applications (2016—Present). Expert Opin Ther Pat 2021, 31 (8), 1-13. https://doi.org/10.1080/13543776.2021.1903430.
Slack, R. J.; Mills, R.; Mackinnon, A. C. The Therapeutic Potential of Galectin-3 Inhibition in Fibrotic Disease. Int J Biochem Cell Biology 2020, 105881. https://doi.org/10.1016/j.biocel.2020.105881.
Sundblad, V., Morosi, L. G., Geffner, J. R. & Rabinovich, G. A. Galectin-1: A Jack-of-All-Trades in the Resolution of Acute and Chronic Inflammation. J. Immunol. 199, 3721-3730 (2017).
Wolf, Y.; Anderson, A. C.; Kuchroo, V. K. TIM3 Comes of Age as an Inhibitory Receptor. Nat Rev Immunol 2020, 20 (3), 173-185. https://doi.org/10.1038/s41577-019-0224-6.
Wu, D.; Kanda, A.; Liu, Y.; Kase, S.; Noda, K.; Ishida, S. Galectin-1 Promotes Choroidal Neovascularization and Subretinal Fibrosis Mediated via Epithelial-Mesenchymal Transition. FASEB J. 2019, 33 (2), 2498-2513. https://doi.org/10.1096/fj.201801227r.
Yang, R.; Sun, L.; Li, C.-F.; Wang, Y.-H.; Yao, J.; Li, H.; Yan, M.; Chang, W.-C.; Hsu, J.-M.; Cha, J.-H.; Hsu, J. L.; Chou, C.-W.; Sun, X.; Deng, Y.; Chou, C.-K.; Yu, D.; Hung, M.-C. Galectin-9 Interacts with PD-1 and TIM-3 to Regulate T Cell Death and Is a Target for Cancer Immunotherapy. Nat Commun 2021, 12 (1), 832. https://doi.org/10.1038/s41467-021-21099-2.

1-15. (canceled)

16. A D-galactopyranose compound of formula (1) 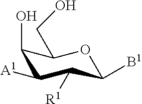
wherein
the pyranose ring is β-D-galactopyranose,
A1 is 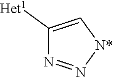
wherein the asterix * indicates the nitrogen atom of the triazole ring that is covalently attached to the galactopyranose;
wherein Het¹ is a five or six membered heteroaromatic ring selected from the group consisting of formulas 2 to 11, wherein the asterix * indicates the carbon or nitrogen atom of the heteroaromatic ring that is covalently attached to the triazole group in formula A¹: 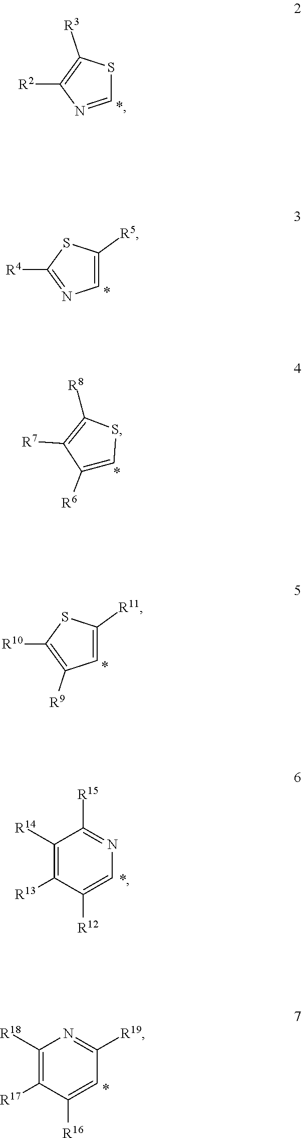 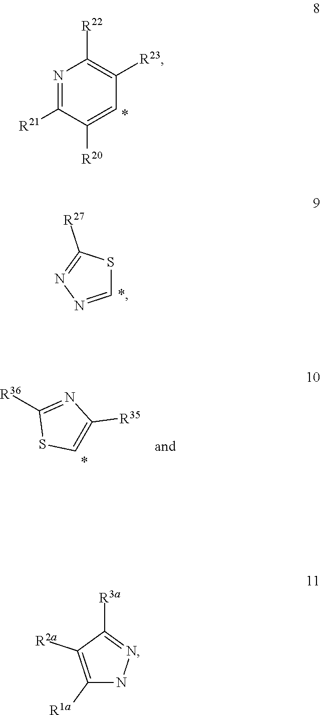
wherein R^{1a}, R^{2a}, R^{3a}, R², R³, R⁴, R⁵ R⁶, R⁷, R⁸, R⁹ R¹⁰, R¹¹, R¹², R¹³, R¹⁴, R¹⁵, R¹⁶, R¹⁷, R¹⁸, R¹⁹, R²⁰, R²¹, R²² and R²³, R²⁷, R³⁵ and R³⁶ are independently selected from H; halogen; OH; CN; SH; S—C₁₋₆ alkyl; C₁₋₆ alkyl, optionally substituted with a F; cyclopropyl, optionally substituted with a F; O-cyclopropyl optionally substituted with a F; OC₁₋₆ alkyl optionally substituted with a F; NR²⁴R²⁵, wherein R²⁴ is selected from H and C₁₋₆ alkyl, and R²⁵ is selected from H, C₁₋₃ alkyl, and C(═O)R²⁶, wherein R²⁶ is selected from H, and C₁₋₆ alkyl; C(═O)NR^{24a}R^{25a}, wherein R^{24a} is selected from H and C₁₋₆ alkyl, and R^{25a} is selected from H, C₁₋₃ alkyl, and C(═O)R^{26a}, wherein R^{26a} is selected from H, and C₁₋₆ alkyl; C(═O)OR^{24b}R^{25b}, wherein R^{24b} is selected from H and C₁₋₆ alkyl, and R^{25b} is selected from H, C₁₋₃ alkyl, and C(═O)R^{26b}, wherein R^{26b} is selected from H, and C₁₋₆ alkyl;
wherein B¹ is a pyrazol substituted with one or more groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R²⁸—C₁₋₆ alkyl, c) C₃₋₆cycloalkyl optionally substituted with one or more of C₁₋₆ alkyl, amino, CN, halogen, or hydroxy, c) C₁₋₆ alkenyl, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R²⁹)(R³⁰)N)C₁₋₆ alkylNH, or (pyridinyl)C₁₋₆ alkylNH, h) (R³¹)(R³²)N, i) C₂-alkynyl, j) R²⁸,
wherein R²⁸ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R³³)(R³⁴)N; orb) (C₁₋₆ alkyl-SO₂)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R²⁹ is hydrogen or C₁₋₆ alkyl;
R₃₀ is hydrogen or C₁₋₆ alkyl; or
(R²⁹)(R³⁰)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R³¹ is hydrogen, C₁₋₆ alkyl, C₁₋₆ alkylcarbonyl, or C₁₋₆ alkylsulfonyl;
R³² is hydrogen or C₁₋₆ alkyl; or
(R³¹)(R³²)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R³³ is hydrogen, C₁₋₆ alkyl, C₁₋₆ alkylcarbonyl, or C₁₋₆ alkylsulfonyl;
R³⁴ is hydrogen or C₁₋₆ alkyl; or
(R³³)(R³⁴)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
k) halogen, and l) cyano;
R¹ is selected from the group consisting of a) OC₁₋₆ alkyl optionally substituted with one or more halogen, phenyl, phenyl substituted with one or more groups selected form OH and halogen, CN, OR³⁷, NR³⁸R³⁹, and CONH₂, wherein R³⁷ is selected from the group consisting of H, CN, a halogen, methyl optionally substituted with a F, OCH₃ optionally substituted with a F, OCH₂CH₃ optionally substituted with a F, OH, and R⁴⁰—CONH— wherein R⁴⁰ is selected from C₁₋₃ alkyl and cyclopropyl, R³⁸ is selected from the group consisting of H, CN, a halogen, methyl optionally substituted with a F, OCH₃ optionally substituted with a F, OCH₂CH₃ optionally substituted with a F, OH, and R⁴¹—CONH— wherein R⁴¹ is selected from C₁₋₃ alkyl and cyclopropyl, and R³⁹ is selected from the group consisting of H, CN, a halogen, methyl optionally substituted with a F, OCH₃ optionally substituted with a F, OCH₂CH₃ optionally substituted with a F, OH, and R⁴²—CONH— wherein R⁴² is selected from C₁₋₃ alkyl and cyclopropyl, b) branched OC₃₋₆ alkyl optionally substituted with one or more halogen, CN, OR⁴³, NR⁴⁴R⁴⁵, and CONH₂, wherein R⁴³ is selected from the group consisting of H, CN, a halogen, methyl optionally substituted with a F, OCH₃ optionally substituted with a F, OCH₂CH₃ optionally substituted with a F, OH, and R⁴⁶—CONH— wherein R⁴⁶ is selected from C₁₋₃ alkyl and cyclopropyl, R⁴⁴ is selected from the group consisting of H, CN, a halogen, methyl optionally substituted with a F, OCH₃ optionally substituted with a F, OCH₂CH₃ optionally substituted with a F, OH, and R⁴⁷—CONH— wherein R⁴⁷ is selected from C₁₋₃ alkyl and cyclopropyl, and R⁴⁵ is selected from the group consisting of H, CN, a halogen, methyl optionally substituted with a F, OCH₃ optionally substituted with a F, OCH₂CH₃ optionally substituted with a F, OH, and R⁴⁸—CONH— wherein R⁴⁸ is selected from C₁₋₃ alkyl and cyclopropyl, c) cyclic OC₃₋₆ alkyl optionally substituted with one or more halogen, CN, OR⁴⁹, NR⁵⁰R⁵¹, and CONH₂, wherein R⁴⁹ is selected from the group consisting of H, CN, a halogen, methyl optionally substituted with a F, OCH₃ optionally substituted with a F, OCH₂CH₃ optionally substituted with a F, OH, and R⁵²—CONH— wherein R⁵² is selected from C₁₋₃ alkyl and cyclopropyl, R⁵⁰ is selected from the group consisting of H, CN, a halogen, methyl optionally substituted with a F, OCH₃ optionally substituted with a F, OCH₂CH₃ optionally substituted with a F, OH, and R⁵³—CONH— wherein R⁵³ is selected from C₁₋₃ alkyl and cyclopropyl, and R⁵¹ is selected from the group consisting of H, CN, a halogen, methyl optionally substituted with a F, OCH₃ optionally substituted with a F, OCH₂CH₃ optionally substituted with a F, OH, and R⁵⁴—CONH— wherein R⁵⁴ is selected from C₁₋₃ alkyl and cyclopropyl, d) OH; or
a pharmaceutically acceptable salt or solvate thereof.

17. The compound of claim 16 wherein Het1 is selected from the group consisting of 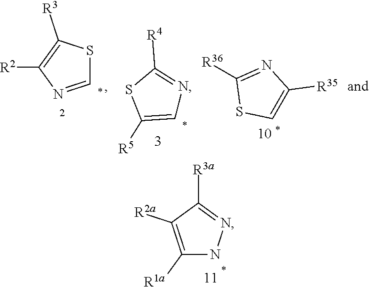
Wherein
R^{1a} is selected from the group consisting of hydrogen, OH, C₁₋₆ alkyl, amino and halogen;
R^{2a} is selected from the group consisting of hydrogen, OH, C₁₋₆ alkyl, amino and halogen; and
R^{3a} is selected from the group consisting of hydrogen, OH, C₁₋₆ alkyl, amino and halogen;
R² is selected from the group consisting of hydrogen, methyl, OH and halogen;
R³ is selected from the group consisting of hydrogen, C₁₋₆ alkyl and halogen;
R⁴ is selected from the group consisting of OH, C₁₋₆ alkyl, halogen and amino;
R⁵ is selected from the group consisting of hydrogen, C₁₋₆ alkyl and halogen;
R³⁵ and R³⁶ are independently selected from hydrogen, C₁₋₆ alkyl, amino and halogen.

18. The compound of claim 16 wherein Het1 is formula 2
wherein R² is selected from the group consisting of hydrogen, methyl, OH and halogen; and
R³ is selected from the group consisting of hydrogen, methyl and halogen.

19. The compound of claim 16 wherein B¹ is a pyrazolyl substituted with one or more groups selected from the group consisting of a) C₁₋₄ alkyl optionally substituted with one or more of C₁₋₆ alkyl, amino, CN, halogen, hydroxy, C₁₋₆ alkoxy, carboxy, alkoxycarbonyl, H₂NCO, b) R²⁸—C₁₋₆ alkyl, c) C₃₋₆cycloalkyl optionally substituted with one or more of C₁₋₆ alkyl, amino, CN, halogen, or hydroxy, c) C₂₋₄ alkenyl, d) C₁₋₆ alkoxy, e) C₁₋₆ alkylthio, f) C₁₋₆ alkylsulfonyl, g) COOH or COOC₁₋₄ alkyl h) (R³¹)(R³²)N, i) C₂-alkynyl, j) R²⁸, k) halogen, l) cyano; wherein R²⁸, R³¹ and R³² are as defined above.

20. The compound of claim 16 wherein B¹ is a pyrazolyl substituted with a phenyl optionally substituted with a group selected from methyl, CF₃, and halogen.

21. The compound of claim 16 wherein B¹ is 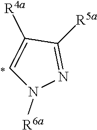
wherein the asterix * indicates the carbon atom of the pyrazol ring that is covalently attached to the galactopyranose,
wherein R^{4a}, R^{5a}, and R^{6a} are independently selected from the group consisting of hydrogen, C₁₋₄ alkyl, C₂₋₄ alkenyl, halogen, cyano, COOH, COOC₁₋₄ alkyl, pyrrolyl, furanyl, thienyl, pyrazolyl, isoxazolyl, isothiazolyl, imidazolyl, oxazolyl, thiazolyl, triazolyl, oxadiazolyl, thiadiazolyl, pyridinyl, benzothiazolyl, phenyl, or indolyl, provided that not all three of R^{4a}, R^{5a}, and R^{6a} are hydrogen at the same time; wherein pyrrolyl, furanyl, thienyl, pyrazolyl, isoxazolyl, isothiazolyl, imidazolyl, oxazolyl, thiazolyl, triazolyl, oxadiazolyl, thiadiazolyl, pyridinyl, benzothiazolyl, phenyl, or indolyl are optionally substituted with a group selected from cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂.

22. The compound of claim 21 wherein R^{4a} is hydrogen, R^{5a} is selected from hydrogen, halogen, C₁₋₃ alkyl, COOH, COOC₁₋₃ alkyl, C₂₋₃ alkenyl, cyano and R^{6a} is selected from a) a phenyl substituted with a group selected from methyl, CF₃, and halogen; b) a pyridinyl substituted with a group selected from methyl, CF₃, and halogen; or c) a benzothiazolyl substituted with a group selected from methyl, CF₃, and halogen.

23. The compound of claim 16 wherein R¹ is selected from OC₁₋₆ alkyl optionally substituted with one or more halogen, phenyl, phenyl substituted with one or more groups selected form OH and halogen, CN, OR³⁷, NR³⁸R³⁹, and CONH₂, wherein R³⁷ is selected from the group consisting of H, CN, a halogen, methyl optionally substituted with a F, OCH₃ optionally substituted with a F, OCH₂CH₃ optionally substituted with a F, OH, and R⁴⁰—CONH— wherein R⁴⁰ is selected from C₁₋₃ alkyl and cyclopropyl, R³⁸ is selected from the group consisting of H, CN, a halogen, methyl optionally substituted with a F, OCH₃ optionally substituted with a F, OCH₂CH₃ optionally substituted with a F, OH, and R⁴¹—CONH— wherein R⁴¹ is selected from C₁₋₃ alkyl and cyclopropyl, and R³⁹ is selected from the group consisting of H, CN, a halogen, methyl optionally substituted with a F, OCH₃ optionally substituted with a F, OCH₂CH₃ optionally substituted with a F, OH, and R⁴²—CONH— wherein R⁴² is selected from C₁₋₃ alkyl and cyclopropyl.

24. The compound of claim 16 wherein R¹ is selected from OC₁₋₆ alkyl optionally substituted with one or more halogen.

25. The compound of claim 16 wherein R¹ is selected from OH or OC₁₋₃ alkyl.

26. The compound of claim 16 selected from any one of the group consisting of:
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
5-Chloro-1-{5-{3-deoxy-2-O-methyl-3-[4-(2-thiazolyl)-1H-1,2,3-triazol-1-yl]-β-D-galactopyranosyl}-1H-1,2-pyrazol-1-yl}-2-(trifluoromethyl)benzene,
5-Chloro-1-{5-{3-deoxy-3-[4-(5-methylthiazol-2-yl)-1H-1,2,3-triazol-1-yl]-2-O-methyl-β-D-galactopyranosyl}-1H-1,2-pyrazol-1-yl}-2-(trifluoromethyl)benzene,
5-Chloro-1-{5-{3-deoxy-3-[4-(4-methylthiazol-2-yl)-1H-1,2,3-triazol-1-yl]-2-O-methyl-β-D-galactopyranosyl}-1H-1,2-pyrazol-1-yl}-2-(trifluoromethyl)benzene,
5-Chloro-1-{5-{3-deoxy-3-[4-(2-thiazolyl)-1H-1,2,3-triazol-1-yl]-β-D-galactopyranosyl}-1H-1,2-pyrazol-1-yl}-2-(trifluoromethyl)benzene,
1-{5-{3-[4-(2-Aminothiazol-4-yl)-1H-1,2,3-triazol-1-yl]-3-deoxy-β-D-galactopyranosyl}-1H-1,2-pyrazol-1-yl}-5-chloro-2-(trifluoromethyl)benzene,
5-Chloro-1-{5-{3-deoxy-3-[4-(2-hydroxythiazol-4-yl)-1H-1,2,3-triazol-1-yl]-β-D-galactopyranosyl}-1H-1,2-pyrazol-1-yl}-2-(trifluoromethyl)benzene,
1-{5-{3-[4-(3-Chloro-1H-1,2-pyrazol-1-yl)-1H-1,2,3-triazol-1-yl]-3-deoxy-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-β-D-galactopyranosyl}-3-methyl-1H-1,2-pyrazol-1-yl}-5-chloro-2-(trifluoromethyl)benzene,
1-{5-{3-[4-(5-Chlorothiazol-2-yl)-1H-1,2,3-triazol-1-yl]-3-deoxy-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-β-D-galactopyranosyl}-1H-1,2-pyrazol-1-yl}-5-chloro-2-(trifluoromethyl)benzene,
1-{5-{3-[4-(5-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-methyl-1H-1,2-pyrazol-1-yl}-5-chloro-2-(trifluoromethyl)benzene,
5-Chloro-1-{5-{3-deoxy-2-O-methyl-3-[4-(2-thiazolyl)-1H-1,2,3-triazol-1-yl]-β-D-galactopyranosyl}-3-methyl-1H-1,2-pyrazol-1-yl}-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-methyl-1H-1,2-pyrazol-1-yl}-2-methylbenzothiazole,
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-2-methylbenzothiazole,
1-{5-{3-[4-(4-Chlorothiazol-2-yl)-1H-1,2,3-triazol-1-yl]-3-deoxy-2-O-methyl-β-D-galactopyranosyl}-3-ethoxycarbonyl-1H-1,2-pyrazol-1-yl}-5-chloro-2-(trifluoromethyl)benzene,
1-{3-Carboxy-5-{3-[4-(4-chlorothiazol-2-yl)-1H-1,2,3-triazol-1-yl]-3-deoxy-2-O-methyl-p-D-galactopyranosyl}-1H-1,2-pyrazol-1-yl}-5-chloro-2-(trifluoromethyl)benzene,
5-Bromo-1-{5-{3-[4-(4-chlorothiazol-2-yl)-1H-1,2,3-triazol-1-yl]-3-deoxy-β-D-galactopyranosyl}-1H-1,2-pyrazol-1-yl}-2-(trifluoromethyl)benzene,
5-Bromo-1-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-4,5-dichloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-4-fluoro-2-(trifluoromethyl)benzene,
5-Bromo-1-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-4-fluoro-2-(trifluoromethyl)benzene,
5-Bromo-1-{5-{3-[4-(4-chlorothiazol-2-yl)-1H-1,2,3-triazol-1-yl]-3-deoxy-β-D-galactopyranosyl}-1H-1,2-pyrazol-1-yl}-4-fluoro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-chloro-1H-1,2-pyrazol-1-yl}-5-chloro-2-(trifluoromethyl)benzene,
1-{3-Bromo-5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-(1-methylethenyl)-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-isopropyl-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-ethenyl-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-ethyl-1H-1,2-pyrazol-1-yl}-5-chloro-2-(trifluoromethyl)benzene,
1-{5-{3-[4-(4-Chlorothiazol-2-yl)-1H-1,2,3-triazol-1-yl]-3-deoxy-2-O-methyl-β-D-galactopyranosyl}-3-cyano-1H-1,2-pyrazol-1-yl}-5-chloro-2-(trifluoromethyl)benzene,
3-{5-{3-[4-(4-Chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)pyridine,
5-Bromo-3-{5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-2-(trifluoromethyl)pyridine,
1-{5-{3-[4-(2-Aminothiazol-4-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene,
5-Chloro-1-{5-{3-deoxy-3-[4-(2-hydroxythiazol-4-yl)-1H-1,2,3-triazol-1-yl]-2-O-methyl-p-D-galactopyranosyl}-1H-1,2-pyrazol-1-yl}-2-(trifluoromethyl)benzene,
4,5-Dichloro-1-{5-{3-deoxy-3-[4-(2-hydroxythiazol-4-yl)-1H-1,2,3-triazol-1-yl]-2-O-methyl-β-D-galactopyranosyl}-1H-1,2-pyrazol-1-yl}-2-(trifluoromethyl)benzene,
5-Bromo-1-{5-{3-deoxy-3-[4-(2-hydroxythiazol-4-yl)-1H-1,2,3-triazol-1-yl]-2-O-methyl-p-D-galactopyranosyl}-1H-1,2-pyrazol-1-yl}-4-fluoro-2-(trifluoromethyl)benzene,
5-Chloro-1-{5-{3-deoxy-2-O-methyl-3-[4-(4-thiazolyl)-1H-1,2,3-triazol-1-yl]-β-D-galactopyranosyl}-1H-1,2-pyrazol-1-yl}-2-(trifluoromethyl)benzene, and
1-{3-Aminocarbonyl-5-{3-[4-(4-chlorothiazol-2-yl)-1H-1,2,3-triazol-1-yl]-3-deoxy-2-O-methyl-β-D-galactopyranosyl}-1H-1,2-pyrazol-1-yl}-5-chloro-2-(trifluoromethyl)benzene; or
a pharmaceutically acceptable salt or solvate thereof.

27. A pharmaceutical composition comprising the compound of claim 16 and optionally a pharmaceutically acceptable additive.

28. A method for treating a disorder relating to the binding of a galectin-1 and/or 3 to a ligand in a mammal, wherein said disorder is selected from the group consisting of inflammation, acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, chronic autoimmune diseases in lung, liver, kidney, heart, skin, muscle, and/or gut, chronic bacterial infections, chronic viral related inflammation; fibrosis, pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust, acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, carcinomas, sarcomas, leukemias and lymphomas, T-cell lymphomas; metastasising cancers; autoimmune diseases, psoriasis, rheumatoid arthritis, Crohn's disease, ulcerative colitis, intestinal fibrosis, ankylosing spondylitis, systemic lupus erythematosus; metabolic disorders; coagulopathies, thrombosis proneness idiopathic (thrombophilia), autoimmune based thrombophilia, microthrombosis at multiorgan failure, COVID-19 related coagulopathy, thrombophilia in cancer disease; cardiovascular disorders, cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, ocular angiogenesis or a disease or condition associated with ocular angiogenesis, neovascularization related to cancer; and eye diseases, age-related macular degeneration and corneal neovascularization; atherosclerosis; endocrine disorders, Addison, autoimmune hypophysitis; metabolic diseases, diabetes; type 2 diabetes; insulin resistance; obesity; Diastolic HF; atrophic diseases in the brain, Alzheimer's and Parkinson's, atrophic diseases in the cerebellum, cerebellar atrophy, atrophic spinal diseases, ALS; disorders related to transplantation in organs, anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis, COPD (Chronic Obstructive Pulmonary Disease) and asthma; Otosclerosis, mesothelioma; post-surgery disorders, anti-keloid, anti-stricture, anti-adhesion, anti-thrombosis, fibrosis/scar reduction following cosmetic procedures; toxin exposure disorders, toxic hepatitis, cholera toxin related, mushroom toxin based acute renal failure, pertussis toxin, Aeromonas hydrophila enterotoxin, cadmium induced cardiac toxicity, Helicobacter O-antigen related toxicity, LPS based toxicity, Streptozotocin toxicity, asbestos exposure, Nephrogenic Systemic Fibrosis (Post Contrast Agents); Tissue injury, Spinal cord injury, Peripheral nerve repair; congenital hepatic fibrosis; hereditary fibrosing poikiloderma with tendon contractures, myopathy, and pulmonary fibrosis; liver disorders, non-alcoholic steatohepatitis (NASH) or non-alcoholic fatty liver disease, liver cirrhosis of various origins, alcoholic and non-alcoholic, autoimmune cirrhosis, primary biliary cirrhosis and sclerosing cholangitis, virally induced cirrhosis, cirrhosis induced by genetic disease; Liver cancer, cholangiocarcinoma, biliary tract cancer; neurodegenerative disorders, Parkinsons disease, Alzheimers disease, cognitive impairment, cerebrovascular diseases, stroke, traumatic brain injury, Huntington's disease, amyotrophic lateral sclerosis (ALS), multiple sclerosis (MS), peripheral nephropathy,
comprising administering a therapeutically effective amount of the compound of claim 16.
